(19) United States
(12) Patent Application Publication
(10) Pub. No.: US 2007/0015150 A1
(43) Pub. Date: Jan. 18, 2007

(54) Novel nucleic acid sequences and proteins of tumors and neoplasias of the thyroid gland

(76) Inventor: Jorn Bullerdiek, Bremen (DE)

Correspondence Address: MCDERMOTT WILL & EMERY LLP, 600 13TH STREET, N.W., WASHINGTON, DC, 20005-3096, US

(21) Appl. No.: 10/512,809

(22) PCT Filed: May 2, 2003
(86) PCT No.: PCT/EP03/04642

§ 371 Date: Oct. 28, 2005

(30) Foreign Application Priority Data
May 10, 2002 (DE) 102 19 413.0
Sep. 14, 2002 (DE) 102 42 705.4

(51) Int. Cl. A61K 48/00; C12Q 1/68; C07H 21/04; C12P 21/06; A61K 39/395; C07K 14/82; C07K 16/30

(52) U.S. Cl. 435/6; 514/44; 424/155.1; 435/69.1; 435/320.1; 435/325; 530/350; 530/388.8; 536/23.5

(57) ABSTRACT

The Invention relates to a nucleic acid and proteins of tumors and neoplasias of the thyroid gland. Said nucleic acid comprises a nucleic acid sequence selected among the group consisting of SEQ ID No. 1, 3, 4, 9 and SEQ ID No. 11 to 16. The invention also relates to fusion proteins observed in tumors and neoplasias of the thyroid gland, said fusion proteins containing an amino acid sequence according to SEQ ID No. 2, SEQ ID No. 5 to 8, 10, and 17.

The present invention relates to nucleic acids with an expression altered by hyperplasias and/or tumors, nucleic acid coding for the human gene DRIP, which is also designated as synonymous to THADA (Thyroid Adenoma Associated), and their tumor specific new combinations and in particular homologens thereof, vectors and cells containing these, polypeptides coded by these, antibodies directed against these, a process for determining compounds suited as tumor therapeutics, a process for determining genes, which are involved in the formation of thyroid gland tumors and uses of said nucleic acids.

The significance of the thyroid gland as a result of its hormone production for controlling the growth and development of the body has been known for a long time already in medicine, as have the perspicuous changes with a functional disturbance of this gland at least in a few cases in the truest sense of the word.

With respect to the pathogenesis of goitre and thyroid gland tumors there has generally been no success particularly at a molecular level in developing a comprehensive presentation.

There are currently two concepts under discussion, whereby one presupposes that the hyperplastic tissue is definitely considered as coming from chronic stimulation by a trophic hormone, the result of which is finally the growth of polyclonal nodes. This concept is designated as non-neoplastic endocrine hyperplasia (NNEH). The second concept is based on the fact that the nodes represent authentic clonal tumors.

In the case of the thyroid gland it has eventuated that the simple concept of non-neoplastic endocrine hyperplasia applicable to other glands cannot be applied. An overview of the considerations currently being made in this area is found at Studer, H. (1995); Endocrine Reviews, Vol. 16, No. 4, pages 411-426 and Derwahl M and Studer H. (2002), Trends Endocrinol. Metab., Vol. 13 No. 1, pages 23-8.

Early diagnosis for the treatment of goitre and thyroid gland tumors is therefore necessary to begin to apply appropriate therapeutic concepts.

The object of the present invention is to provide means for diagnosis and therapy of functional disturbances of the thyroid gland, hyperplasias of the thyroid gland and tumors of the thyroid gland at the molecular level, whereby in particular nucleic acid sequences are to be provided, which are involved in the pathogenetic mechanisms and are also suited to examine these further, as well as those which have an affect on the pathogenesis mechanisms or which can influence them.

In addition, drugs based thereon and generally pharmaceutical compounds are to be provided.

Furthermore kits for diagnosis and/or therapy of functional disturbances of the thyroid gland, hyperplasias of the thyroid gland and tumors of the thyroid gland as well as processes for proving the latter are to be made available.

In terms of the invention the object is solved by a nucleic acid with an expression altered by hyperplasias and/or tumors, whereby the nucleic acid comprises a nucleic acid sequence, which is selected from the group comprising SEQ. ID. No 1, SEQ. ID. No 3, SEQ. ID. No. 4, SEQ. ID. No 9, and SEQ. ID. No 11 to 16 or in each case a part or a fragment thereof. (Here SEQ. ID. No. 11 to SEQ. ID. No. 16 means all sequences from 11 to 16, i.e. SEQ. ID. No. 11, SEQ. ID. No. 12, SEQ. ID. No. 13 etc., to SEQ. ID. No. 16). In connection with the present invention inventive nucleic acids are also those which comprise an exon or the transition area between two exons. The transition area can be so short, as is necessary, to enable specific proof the transition area, in particular for diagnostic application.

The corresponding lengths are known to the expert in the area. A transition area can be one between two exons of a gene but also one between two exons of two different genes, as this applies for example to the herein disclosed fusion genes, herein also designated synonymously as fusion transcripts. At the same time with the genes and the fusion genes in particular that area or those exons can be used preferably for diagnosis of the presence of herein described tumors and neoplasias of the thyroid gland, which are arranged in the respective breakpoint region, as is disclosed herein.

In an embodiment it is provided that the tumor is selected from the group which comprises epithelial tumors with a change to the chromosomal band 2p and tumors with a change to the chromosomal band 2p 21-22.

In a further embodiment it is provided that the hyperplasia is selected from the group which comprises hyperplasias of the thyroid gland.

In yet another embodiment it is provided that SEQ. ID. No. 11 codes for DRIP, in particular human DRIP, or a part thereof.

In a further embodiment it is provided that SEQ. ID. No. 12 codes for DRIP, in particular human DRIP, or a part thereof.

In a further embodiment it is provided that SEQ. ID. No. 16 codes for DRIP, in particular human DRIP, or a part thereof.

In a further embodiment it is provided that SEQ. ID. No. 1 for the inventive fusion protein or a part thereof codes.

The inventive fusion protein is also designated herein as DRIP-FUS Ib.

In a further embodiment it is provided that SEQ. ID. No. 9 codes for the inventive fusion protein or a part thereof.

The inventive fusion protein is also designated herein as DRIP-FUS IIa.

In a further embodiment it is provided that SEQ. ID. No. 13 codes for the inventive fusion protein or a part thereof.

The inventive fusion protein is also designated herein as DRIP-FUS I.

In a further embodiment it is provided that SEQ. ID. No. 14 codes for the inventive fusion protein or a part thereof.

The inventive fusion protein is also designated herein as DRIP-FUS Ia.

In a further embodiment it is provided that SEQ. ID. No. 15 codes for the inventive fusion protein or a part thereof.

The inventive fusion protein is also designated herein as DRIP-FUS II.

In a further embodiment it is provided that SEQ. ID. No. 3 codes for FUS I, in particular human FUS I, or a part thereof.

In a further embodiment it is provided that SEQ. ID. No. 4 codes for FUS II, in particular human FUS II, or a part thereof.

In yet a further aspect the object according to the present invention is solved by a nucleic acid comprising a nucleic acid sequence, which without the degeneration of the genetic code would code for the same amino acid sequence as one of the inventive nucleic acids.

In a further aspect the object according to the present invention is solved by a nucleic acid, which hybridises on one of the inventive nucleic acids, in particular under stringent conditions.

In yet a further aspect the object according to the present invention is solved by a vector, whereby the vector comprises one of the inventive nucleic acids.

In an embodiment it is provided that the vector further comprises at least one element, selected from the group comprising promoters, terminators and enhancers.

In a further embodiment it is provided that the vector is an expression vector.

In yet a further embodiment it is provided that at least a promoter in the in-frame with at least a part coding for a polypeptide is one of the inventive nucleic acids.

In a further aspect the object according to the present invention is solved by a polypeptide coded by one of the inventive nucleic acids.

In yet another aspect the object according to the present invention is solved by a polypeptide with an amino acid sequence according to one of the sequences according to SEQ. ID. No. 2, SEQ. ID. No.5 to SEQ. ID. No. 8, SEQ. ID. No. 10 and SEQ. ID. No. 17.

In an embodiment it is provided that the polypeptide is modified.

In a further aspect the object according to the present invention is solved by a cell, in particular an isolated cell, which comprises an inventive vector.

In a further aspect the object according to the present invention is solved by an antibody, whereby the antibody is directed against an inventive polypeptide.

In an embodiment it is provided that the antibody is directed against one of the inventive nucleic acids.

In a further aspect the object according to the present invention is solved by a ribozyme, whereby the ribozyme is directed against one of the inventive nucleic acids.

In an embodiment it is provided that the ribozyme comprises at least part of one of the inventive nucleic acids or of a nucleic acid substantially complementary thereto.

In a further aspect the object according to the present invention is solved by an antisense nucleic acid comprising a sequence, which corresponds substantially to the whole or partial sequence or is substantially complementary to one of the inventive nucleic acid sequences. As used herein the term “substantially” designates independently of the respective context, in which it is used, homology of at least 70%, preferably at least 80% and preferably at least 90%.

In yet a further aspect the object according to the present invention is solved by an interfering RNA, herein also designated as RNAi, preferably by a small interfering RNA, comprising a sequence which is substantially complementary or substantially identical to one of the nucleic acids as claimed in any one of Claims 1 to 8 or part thereof, whereby the RNAi preferably comprises an area with a length of 21 to 23 nucleotides, which is substantially complementary or substantially identical.

In a further aspect the object according to the present invention is solved by a primer or a nucleic acid probe comprising a nucleic acid, whereby the nucleic acid is substantially complementary or substantially identical is to one of the inventive nucleic acids or a part thereof, whereby the primer and/or the nucleic acid probe comprises a length of 12 to 32 nucleotides, preferably of 14 to 28 nucleotides and preferably of 20 to 28 nucleotides.

In a further aspect the object according to the present invention is solved by a process for determining a compound, which influences and in particular inhibits the effect of a translation product of an inventive nucleic acid according to one of the foregoing claims, comprising the following steps:
provision of the translation product and the compound,
bringing the translation product and the compound into contact in a system, which represents the effect of the translation product, and
determining whether a change in the effect of the translation product occurs under the influence of the compound.

In an embodiment the compound is a cancer or tumor agent.

In a further aspect the object according to the present invention is solved by a process for determining a compound, which influences, in particular inhibits, the effect of a transcription product of one of the inventive nucleic acids comprising the following steps:
provision of the transcription product and the compound,
bringing the transcription product and the compound into contact in a system, which represents the effect of the transcription product, and
determining whether a change in the effect of the transcription product occurs under the influence of the compound.

In an embodiment the compound is a cancer or tumor means.

In an embodiment it is provided that the system is selected from the group, which comprises cellular expression systems, cell-free expression systems, assay for determining the interaction between compound and translation products, and assay for determining the interaction between compound and transcription products.

In a further aspect the object according to the present invention is solved by a process for determining genes, which are responsible for the occurrence of hyperplasias and tumors, in particular of the thyroid gland, comprising the following steps:
detecting the break points in chromosomal translocations of the hyperplasias and the tumors,
determining genes, which lie inside an area of approximately 400 kbp, preferably approximately 320 kB, preferably approximately 150 kbp, in every direction from the breakpoint region, and
determining whether the translation/transcription of the gene in a cell of the hyperplasia or of the tumor is changed relative to a non-hyperplasia cell or a non-tumor cell.

In yet a further aspect the object according to the present invention is solved through use of one of the inventive nucleic acids, of the inventive ribozyme, the inventive antisense-nucleic acid, the inventive RNAi and/or the inventive nucleic acid probe for diagnosis, in particular in vitro, and/or therapy of functional disturbances of the thyroid gland and/or hyperplasias of the thyroid gland and/or tumors, in particular thyroid gland tumors.

In yet a further aspect the object according to the present invention is solved through the use of one of the inventive nucleic acids, the inventive ribozyme, the inventive antisense-nucleic acid, the inventive RNAi and/or the inventive nucleic acid probe for manufacturing a drug, in particular for therapy and/or prevention of functional disturbances of the thyroid gland and/or hyperplasias of the thyroid gland and/or tumors, in particular thyroid gland tumors.

In a further aspect the object according to the present invention is solved through the use of one of the inventive polypeptides for diagnosis, in particular in vitro, and/or therapy of functional disturbances of the thyroid gland and/or hyperplasias of the thyroid gland and/or tumors, in particular thyroid gland tumors.

In yet a further aspect the object according to the present invention is solved through the use of one of the inventive polypeptides for manufacture of a drug.

In a further aspect the object according to the present invention is solved through the use of one of the inventive antibodies for diagnosis, in particular in vitro, and/or therapy of functional disturbances of the thyroid gland and/or hyperplasias of the thyroid gland and/or tumors, in particular thyroid gland tumors.

In a further aspect the object according to the present invention is solved through the use of one of the inventive antibodies for manufacture of a drug, in particular for therapy and/or prevention of functional disturbances of the thyroid gland and/or hyperplasias of the thyroid gland and/or tumors, in particular thyroid gland tumors.

In yet a further aspect the object according to the present invention is solved by a kit for diagnosis of functional disturbances of the thyroid gland and/or hyperplasias of the thyroid gland and/or thyroid gland tumors, characterised in that the kit comprises at least one element, selected from the group which comprises a nucleic acid, a vector, a polypeptide, a cell, an antibody, a ribozyme, an antisense-nucleic acid, RNAi, a primer, and/or a nucleic acid probe, in each case as claimed in any one of the foregoing claims.

In yet a further aspect the object according to the present invention is solved by a process for proving functional disturbances of the thyroid gland and/or hyperplasias of the thyroid gland and/or tumors of the thyroid gland, comprising the following steps:

contacting thyroid gland material with the agent, selected from the group which comprises a nucleic acid, a vector, a polypeptide, an antibody, a ribozyme and a cell, in each case according to the present invention, and determining whether functional disturbances of the thyroid gland and/or hyperplasias of the thyroid gland and/or tumors of the thyroid gland are present.

In an embodiment it is provided that the thyroid gland material is present ex vivo.

In an embodiment it is provided that the thyroid gland material is part of a cytological preparation.

In yet a further aspect the object according to the present invention is solved through the use of one of the inventive nucleic acids and of a part thereof as primer and/or as probe.

In yet a further aspect the object according to the present invention is solved by a primer for displaying and/or screening and/or detecting an inventive nucleic acid, and the primer or the probe is substantially complementary or substantially identical to a part of one of the inventive nucleic acid sequences.

In yet a further aspect the object according to the present invention is solved by the process for displaying a nucleic acid, which comprises a sequence, which can be proven in thyroid gland tumors or goitre, in which translocation with fracture point is in the chromosomal band 2p21-22, whereby the sequence lies inside the chromosomal band 2p21-22, whereby the process comprises the steps:
providing an inventive primer for carrying out a polymerase chain reaction,
providing a nucleic acid sequence taken from the band 2p21-22 of the human chromosome 2 or one of the inventive nucleic acids,
mixing the nucleic acid sequence or the nucleic acid with the primers,
carrying out a polymerase chain reaction.

In yet a further aspect the object according to the present invention is solved by a pharmaceutical composition, comprising: at least one agent, selected from the group which comprises a nucleic acid, a vector, a polypeptide, a cell, an antibody, an antisense-nucleic acid, RNAi, a primer and/or a nucleic acid probe and/or a ribozyme, in each case according to the present invention, as well as combinations thereof, and at least a pharmaceutically acceptable carrier.

In yet a further aspect the object according to the present invention is solved by a process for treatment and/or prophylaxis of tumors and hyperplasias, whereby it is provided that a compound is administered to a patient, in particular a patient who suffers from a tumor, in particular a thyroid gland tumor or hyperplasia, which hinders the effects of the changed expression of one of the inventive nucleic acids.

At the same time the change can be both an increase of the expression and also a decrease of the expression.

In a further aspect the object according to the present invention is solved through the use of a compound, which hinders the effects of the changed expression of one of the inventive nucleic acids, for the manufacture of a drug.

In an embodiment it is provided that the drug is or is employed for treatment and/or prophylaxis of tumors, in particular tumors of the thyroid gland, and/or hyperplasias.

In a further aspect the object according to the present invention is solved through the use of a nucleic acid having a sequence selected from the group comprising SEQ. ID. No. 1, 3, 4, 9 and SEQ. ID. No. 11 to 16, or derivatives thereof and/or polypeptides coded therefrom or derivatives thereof for manufacture of a drug, in particular for treatment of functional disturbances of the thyroid gland and/or hyperplasias of the thyroid gland and/or thyroid gland tumors and/or for manufacture of a diagnostic agent, in particular for the diagnosis of functional disturbances of the thyroid gland and/or hyperplasias of the thyroid gland and/or thyroid gland tumors.

In an embodiment it is provided that the polypeptide has an amino acid sequence according to No. 2, SEQ. ID. No. 5 to 8, 10 and 17.

In an embodiment it is provided that the nucleic acid would hybridise with the nucleic acid according to one of SEQ. ID. No. 1, 3, 4, 9 and SEQ. ID. No. 11 to 16 without the degeneration of the genetic code.

In a further aspect the object according to the present invention is solved through the use of a polypeptide with a sequence, whereby the sequence is selected from the group, which comprises SEQ. ID. No. 2, SEQ. ID. No. 5 to 8, 10 and 17, or derivatives thereof for manufacture of a drug, in particular for treatment of functional disturbances of the thyroid gland and/or hyperplasias of the thyroid gland and/or thyroid gland tumors and/or for manufacture of a diagnostic agent, in particular for the diagnosis of functional disturbances of the thyroid gland and/or hyperplasias of the thyroid gland and/or thyroid gland tumors.

In a further aspect the object according to the present invention is solved by a process for screening an agent for treatment of functional disturbances of the thyroid gland and/or hyperplasias of the thyroid gland and/or tumors, in particular thyroid gland tumors, and/or of a diagnostic means for the diagnosis of functional disturbances of the thyroid gland and/or hyperplasias of the thyroid gland and/or tumors, in particular thyroid gland tumors, comprising the steps:
providing a candidate compound,
providing an expression system and/or activity system;
bringing the candidate compound in contact with the expression system and/or the activity system;
determining whether under the influence of the candidate compound the expression and/or the activity of a nucleic acid with a sequence, whereby the sequence is selected from the group, comprises SEQ. ID. No. 1, 3, 4, 9 and SEQ. ID. No. 11 to 16, or derivatives thereof and/or polypeptides coded therefrom and/or polypeptides with a sequence according to SEQ. ID. 2, SEQ. ID. No. 5 to 8, 10 and 17 derivatives thereof is changed.

In an embodiment it is provided that the candidate compound is contained in a compound library.

In an embodiment it is provided that the candidate compound is selected from the group of compound classes, which comprises peptides, proteins, antibodies, anticalins, functional nucleic acids and small molecules.

In an embodiment it is provided that the functional nucleic acids are selected from the group comprising aptamers, aptazymes, ribozymes, spiegelmers, antisense-oligonucleotides and RNAi.

In a further aspect the object according to the present invention is solved through use of a nucleic acid with a sequence, whereby the sequence is selected from the group, which comprises SEQ. ID. No. 1, 3, 4, 9 and SEQ. ID. No. 11 to 16, or derivatives thereof and/or polypeptides coded therefrom or derivatives thereof and/or a polypeptide with a sequence according to SEQ. ID. No. 2, SEQ. ID. No. 5 to 8, 10 and 17 or of a derivative thereof and/or in particular of a natural interaction partner of a nucleic acid with a sequence, whereby the sequence is selected from the group, which comprises SEQ. ID. No. 1, 3, 4, 9 and SEQ. ID. No. 11 to 16, or derivatives thereof and/or polypeptides coded therefrom or derivatives thereof and/or a nucleic acid coding therefor and/or the interaction partner of a polypeptide with a sequence according to SEQ. ID. No. 2, SEQ. ID. No. 5 to 8, 10 and 17 or of a derivative thereof as target molecule for the development and/or manufacture of a diagnostic means for diagnosis of functional disturbances of the thyroid gland and/or hyperplasias of the thyroid gland and/or thyroid gland tumors, and/or for the development and/or manufacture of a drug for prevention and/or treatment of functional disturbances of the thyroid gland and/or hyperplasias of the thyroid gland and/or tumors, in particular tumors of the thyroid gland.

In an embodiment it is provided that the drug or the diagnostic agent comprises an agent selected from the group, which comprises antibodies, peptides, anticalins, small molecules, antisense-molecules, aptamers, spiegelmers and RNAi molecules.

In an embodiment it is provided that the agent interacts with a nucleic acid having a sequence, whereby the sequence is selected from the group, which comprises SEQ. ID. No. 1, 3, 4, 9 and SEQ. ID. No. 11 to 16, or derivatives thereof and/or with a nucleic acid coding for a natural interaction partner in particular, interacts in particular with mRNA, genomic nucleic acid or cDNA.

In a further aspect the object according to the present invention is solved through use of a polypeptide, which interacts with a peptide, coded by a nucleic acid with a sequence, whereby the sequence is selected from the group, which comprises SEQ. ID. No. 1, 3, 4, 9 and SEQ. ID. No. 11 to 16, or derivatives thereof and/or a polypeptide according to SEQ. ID. No. 2, SEQ. ID. No. 5 to 8, 10 and 17 and/or interacts with a natural interaction partner thereof in particular for development or manufacture of a diagnostic means for diagnosis of functional disturbances of the thyroid gland and/or hyperplasias of the thyroid gland and/or thyroid gland tumors, and/or for development or manufacture of a drug for prevention and/or treatment of functional disturbances of the thyroid gland and/or hyperplasias of the thyroid gland and/or tumors, in particular thyroid gland tumors.

In an embodiment it is provided that the polypeptide is selected from the group comprising antibodies and binding polypeptides.

In a further aspect the object according to the present invention is solved through the use of a nucleic acid, which interacts with a polypeptide, whereby the polypeptide is coded by a nucleic acid with a sequence, whereby the sequence is selected from the group, which comprises SEQ. ID. No. 1, 3, 4, 9 and SEQ. ID. No. 11 to 16, or derivatives thereof and/or a polypeptide according to SEQ. ID. No. 2, SEQ. ID. No. 5 to 8, 10 and 17 and/or with a natural interaction partner thereof in particular for development or manufacture of a diagnostic means for diagnosis of functional disturbances of the thyroid gland and/or hyperplasias of the thyroid gland and/or thyroid gland tumors, and/or for development or manufacture of a drug for prevention and/or treatment of functional disturbances of the thyroid gland and/or hyperplasias of the thyroid gland and/or tumors, in particular thyroid gland tumors.

In an embodiment it is provided that the nucleic acid is selected from the group, which comprises aptamers and spiegelmers.

In a further aspect the object according to the present invention is solved through the use of a first nucleic acid, which interacts with a second nucleic acid, whereby the second nucleic acid has a sequence, whereby the sequence is selected from the group, which comprises SEQ. ID. No. 1, 3, 4, 9 and SEQ. ID. No. 11 to 16, or derivatives thereof and/or interacts with a nucleic acid, which codes for an interaction partner of a polypeptide with a sequence according to SEQ. ID. No. 2, SEQ. ID. No. 5 to 8, 10 and 17 for development or manufacture of a drug for prevention and/or treatment of functional disturbances of the thyroid gland and/or hyperplasias of the thyroid gland and/or tumors, in particular thyroid gland tumors.

In an embodiment it is provided that the interacting first nucleic acid is an antisense-oligonucleotide, a ribozyme and/or RNAi.

In an embodiment of the last two aspects it is provided that the second nucleic acid is the respective cDNA or mRNA.

In a further aspect the object according to the present invention is solved by a pharmaceutical composition comprising at least an agent, selected from the group, as defined by the different inventive uses, and at least a pharmaceutically acceptable carrier, in particular for prevention and/or treatment of functional disturbances of the thyroid gland and/or hyperplasias of the thyroid gland and/or tumors, in particular thyroid gland tumors.

In a further aspect the object according to the present invention is solved by a kit for characterising the status of the thyroid gland, comprising at least an agent, defined by one of the inventive uses.

In a further aspect the object according to the present invention is solved by a polypeptide comprising an amino acid sequence according to SEQ. ID. No. 2, SEQ. ID. No. 5 to 8, 10 and 17 or a functional fragment thereof.

In a further aspect the object according to the present invention is solved through use of the inventive polypeptide according to one of the inventive uses.

Further aspects and embodiments of the invention will emerge from the claims.

The surprising knowledge of the present invention is that there is a series of genes or nucleic acid sequences, whereof the expression modified relative to normal tissue can be compounded with the appearance of tumors and hyperplasias and seems to be connected thereto causally.

Within the scope of the present invention it was established that in the case of certain hyperplasias or tumors the result is a change in the expression of specific genes or sequences. Change should be understood here in particular as either an increase in expression or a decrease in expression. The extent of the expression of the gene in question or the sequence in question in cells or tissue, which is or are different to cells of hyperplasias or tumors, serves as reference in this respect.

Within the scope of the present invention it was further established that in the case of specific hyperplasias or tumors this lead to a change in the nucleotide sequence of specific genes or sequences with respect to normal tissue. Such sequences, herein also designated as fusion genes, fusion proteins or fusion transcripts, are for example the herein disclosed DRIP-FUS 1 and DRIP-FUS II with their respective splice variants, or the nucleic acids and amino acid sequences coding for them.

Finally, within the scope of the present invention it was also established that cells from thyroid gland tumors and also cultivated cells from thyroid gland tumors often have a break at chromosome 2p21-22. Here the breakpoint region could be restricted to 316 kbp. 206 kbp of the DRIP gene lie in the breakpoint region (synonymous also as break point cluster). More exactly, the exons 27 to 38 are in the breakpoint region. The 3′ end of the DRIP gene lies to the telomer of the chromosome 2p. The DNA of the DRIP gene is 365186 bp long and has a total of 38 exons. The mRNA sequence and different splice variants are specified in the different SEQ. ID. Nos.

The herein disclosed sequence of the DRIP gene contains a series of coding sequences (English=CDS). Several possible coding sequences can be determined, whereby the first AUG of the specified DRIP mRNA is favoured as translation start, which represent all inventive nucleic acids or amino acid sequences and peptides or proteins in terms of the present invention. An interaction partner of the DRIP gene or of the polypeptide coded therefrom is represented by the so-called death receptor (English death receptor).

As long as reference is made to “stringent conditions” these are conditions as described herein, in particular in the examples, in connection with PCR, Northern Blot and Southern Blot.

Within the scope of the present invention in the sequence protocol mRNA sequences can also be represented as DNA sequences. Further to this, coding sequences (CDS) can also be represented in the form of the translated amino acid sequence.

An area of genomic DNA of 506 kbp was examined to determine the genomic structure of the break point cluster. The BAC clones illustrated in Table 1 were employed for this purpose and analysed.

TABLE 1
Examined BAC clones at the breakpoint region.
The more precise origin of the BAC clones (e.g. library
etc.) is evident from the gene bank entries.

| | Accession no. | Size/fully |
|---|---|---|
| Clone name | in gene bank | sequenced |
| 339H12 | AC010883 | 215532 bp/yes |
| 183F15 | AC092838 | 112424 bp/yes |
| 204D19 | AC092615 | 180345 bp/yes |

206 kbp of the herein disclosed DRIP genes lie in the breakpoint region, whereby more exactly the exons 27 to 38 lie in the breakpoint region. The 3′ end of the DRIP gene lies to the telomer.

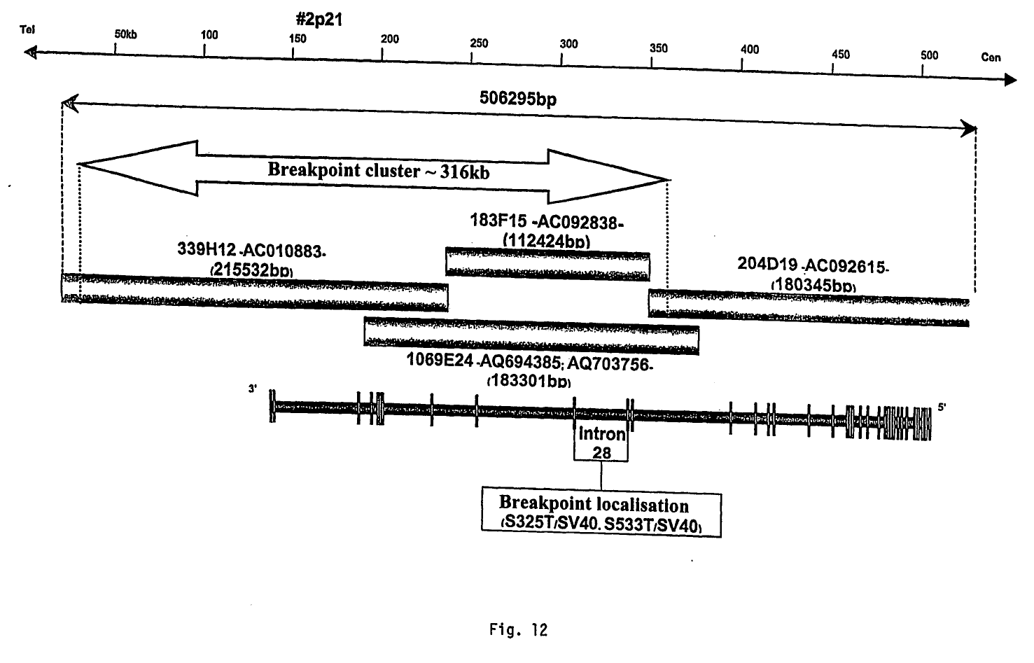

An illustration of the break point region is shown in FIG. 12.

The DRIP Gene

Genomic Organisation of the DRIP Gene

Genomic Size of the DRIP Gene

The DRIP gene is 365186 bp large and fully displayed in SEQ. ID. No. 12. In Table 2 the exon and intron sizes and their relative position to specific clones are laid out.

Herein the formulations Seq. ID. No. and SEQ. ID. No. are also used.

TABLE 2
Size details of the DRIP gene.

| | | | | | mRNA or EST name |
|---|---|---|---|---|---|
| | | | | | (region of each exon or |
| | | Size | | BAC clone, | intron of this table |
| Exon No. | Intron No. | [in bp] | | name in which | line is homologous) |
| 1 | | 108 | | 204D19 | a1832141 (2-109) |
| | 1 | 3514 | | 204D19 | |
| 2 | | 100 | | 204D19 | a1832141 |
| | 2 | 270 | | 204D19 | |
| 3 | | 95 | | 204D19 | a1832141 |
| | 3 | 997 | | 204D19 | |
| 4 | | 131 | | 204D19 | a1832141 |
| | 4 | 3821 | | 204D19 | |
| 5 | | 149 | | 204D19 | a1832141 |
| | 5 | 119 | | 204D19 | |
| 6 | | 33 | | 204D19 | AL832141 |
| | 6 | 252 | | 204D19 | |
| 7 | | 49 | | 204D19 | AL832141 |
| | 7 | 4471 | | 204D19 | |
| 8 | | 188 | | 204D19 | AL832141 |
| | 8 | 3133 | | 204D19 | |
| 9 | | 95 | | 204D19 | AL832141 |
| | 9 | 1278 | | 204D19 | |
| 10 | | | | 204D1 | AL832141 |
| | 10 | 204D19 | | 204D1 | |
| 11 | | | | 204D1 | AL832141 |
| | 1 | 1343 | | 204D1 | |
| 12 | | 179 | | 204D1 | AL832141 |
| | 12 | 951 | | 204D1 [symbol] | |
| 13 | | 156 | | 204D1 | AL832141 |
| | 1 | 1196 | | 204D1 | |
| 14 | | 123 | | 204D1 | AL832141 |
| | 1 | 3566 | | 244D1 | |
| 15 | | 124 | bp | 204D1 [symbol] | AL832141 |
| | | | | | [symbol] |
| | 1 | 6312 | bp | 204D1 | |
| 16 | | 152 | bp | 204D1 | FLJ21792 (2S3-404) |
| | 1 | 3678 | bp | 204D1 | |
| 17 | | 211 | bp | 204D1 | FLJ21792 (405-615) |
| | 1 | 4005 | bp | 204D1 | |
| 18 | | 136 | bp | 204D1 | FLJ21792 (616-751) |
| | 1 | 278 | bp | 204D1 | |
| 19 | | 137 | bp | 204D1 | FL321792 (752-888} |
| | 1 | 2420 | bp | 204D1 | |
| 20 | | 159 | bp | 204D1 | FLJ21792 (889-1047) |
| | 2 | 7893 | bp | 204D1 | |
| 21 | | 158 | bp | 204D1 | FLJ21792 (1048-1205) |
| | 2 | 13170 | | 204D1 | |
| 22 | | 1270 | | 204D19 | KLAA1767 (48-1317) |
| long | | | | | FLJ21877, 3881bp (27-1296) |
| variant | | | | | |
| 22 | | | | | |
| | 2 | 19098 | bp | 204D19 | |
| 23 | | 133 | bp | 204D19 | KIA.A1767 (1318-1450) |
| | | | | | PLJ21877, 3881bp |
| | | | | | (1297-1429) |
| | | | | | FLJ21792 (1316-1448) |
| | 2 | 2912 | bp | 204D19 | |
| 24 | | 114 | bp | 204D19 | KIAA1767 (1451-1564) |
| | | | | | FLJ21877, 3881bp |
| | | | | | (1430-1543) |
| | | | | | FLJ21792 (1449-1562) |
| | 2 | 6666 | bp | 204D 19 | |
| 25 | | 123 | bp | 204D19 | KIAA1767 (1565-1687) |
| | | | | | FLJ21877, 388Ibp |
| | | | | | (1544-1666) |
| | | | | | FLJ21792 (1563-1685) |
| | | 13507 | bp | 204D19 | |
| 26 | | 92 | bp | 204D19 | KIA.A.1767 (1688-1779) |
| | | | | | FLJ21877, 3881bp |
| | | | | | (1667-1758) |
| | | | | | FLJ21792 (1686-1777) |
| | 26 | 54931 | bp | 204D191 | |
| | | | | 183F15 | |
| 27 | | 90 | bp | 183F15 | KIAA1767 (1780-1869) |
| | | | | | FL, J21877, 38$lbp |
| | | | | | (1759-1848) |
| | | | | | FLJ21877, 2I96bp (50-139) |
| | 27 | 1981 | bp | 183F15 | |
| 28 | | 132 | bp | 183F15 | KIAA1767 (1870-2001) |
| | | | | | FLJ21877, 3881bp |
| | | | | | (1849-1980) |
| | | | | | FLJ21877, 2196bp (140-271) |
| | | | | | AF323176, cds (1-128) |
| | | | | | FLJ21877, 2125bp (93-224) |
| | 2 | 29960 | bp | 183F15 | |
| 29 | | 169 | bp | 183F15 | KIAA1767 (2002-2170) |
| | | | | | FLJ21877, 3881bp |
| | | | | | (1981-2149) |
| | | | | | FI, J21792 (1778-1946) |
| | | | | | FLJ21877, 2196bp (272-440) |
| | | | | | AF323176, cds (129-297) |
| | | | | | FLJ21877, 2125bp (225-393) |
| | 29 | 53733 | bp | 183F15 | |
| 30 | | 116 | bp | 183F15 | KIIAA1767 (2171-2286) |
| | | | | | FLJ21877, 3881bp (2150-2265) |
| | | | | | FLJ21792 (1947-2062) |
| | | | | | FLJ2I877, 2196bp (44I- |
| | | | | | 556) |
| | | | | | AF323176, cds (298-413) |
| | | | | | FLJ21877, 2125bp (394-509) |
| | 30 | 116587 | bp | 183F157 | |
| | | | | 339H12 | |
| 31 | | 95 | bp | 183F15 | KIAA1767 (2287-2381) |
| | | | | 339HI2 | FLJ2I877, 388Ibp (2266-2360) |
| | | | | | FLJ21792 (2063-2157) |
| | | | | | FLJ21877, 21966p (SS7- |
| | | | | | 651) |
| | | | | | AF323176 (414-508) |
| | | | | | FLJ21877, 2125bp (510-604) |
| | 31 | 27232 | bp | 183FI57 | |
| | | | | 3391412 | |
| 32 | | 380 | bp | 183F15 | KIAA1767 (2382-2761) |
| | | | | 339H12 | FLJ21877, 3881bp (2361-2740) |
| | | | | | FL321792 (2158-2537) |
| | | | | | FLJ21877, 2196bp (652-1031) |
| | | | | | AF323176 (509-888) |
| | | | | | FLJ21877, 2125bp (605-984) |
| | 32 | 611 | bp | 183F157 | |
| | | | | 339H12 | |
| 33 | | 119 | bp | 183F15 | KIAA1767 (2762-2880) |
| | | | | 3391112 | FLJ21877, 3881bp (2741-2859) |
| | | | | | FLJ21792 (2538-2656) |
| | | | | | FLJ21877, 2196bp (1032-1150) |
| | | | | | AF323176 (889-1007) |
| | | | | | FLJ21877, 2125bp (985-1103) |
| | 33 | 335 | bp | 183F157 | |
| | | | | 339H12 | |
| 34 | | 73 | bp | 183F15 | KIA.A1767 (2881-2953) |
| | | | | 339H12 | FLJ21877, 3881bp (2860-2932) |
| | | | | | FLJ21792 (2657-2729) |
| | | | | | FLJ21877, 2196bp (1151-1223) |
| | | | | | AF323176 (1008-1080) |
| | | | | | FLJ21877, 2125bp (1104-1176) |
| | 34 | 4634 | bp | 183F151 | |
| | | | | 339H12 | |
| 35 | | 154 | bp | 339H12 | KIAA1767 (2954-3107) |
| | | | | | FLJ21877, 3881bp {2933- |
| | | | | | 308b} |
| | | | | | FLJ21792 (2730-2883) |
| | | | | | FLJ21877, 2196bp (1224-1377) |
| | | | | | AF323176 (1081-1234) |
| | | | | | FLJ21877, 2125bp (1177-1330) |
| | | 7011 | bp | 339H12 | |
| 36 | | 132 | bp | 339H12 | KIAA1767 (3108-3239) |
| | | | | | FLJ21877, 3881bp (3088-3219) |
| | | | | | FI, J21792 (2884-3015) |
| | | | | | FLJ21877, 2196bp (1378-1509) |
| | | | | | AF323176 (1235-1366) |
| | | | | | FLJ21877, 2125bp (1332-1463) |
| | 36 | 46882 | bp | 334H12 | |
| 37 | | 170 | bp | 339H12 | KIAA1767 (3240-3409) |
| | | | | | FLJ21877, 3881bp |
| | | | | | (3220-3389) |
| | | | | | FLJ21792 (3016-3185) |
| | | | | | FLJ21877, 2196bp |
| | | | | | (15I0-1679) |
| | | | | | AF323176 (1367-1536) |
| | | | | | FLJ21877, 2125bp |
| | | | | | (1464-1633) |
| | 37 | 1369 | bp | 339H12 | |
| 38 | | 492 | bp | 339H12 | KIAA1767 (3410-3901) |
| | | | | | FLJ21877, 388ibp |
| | | | | | (3390-3881) |
| | | | | | FL321792 (3186-3677) |
| | | | | | FLJ21877, 2196bp |
| | | | | | (1680-217 I) |
| | | | | | AF323176 (1537-1932) |
| | | | | | FL, 121877, 2125bp |
| | | | | | (1634-2125) |

| | |
|---|---|
| length | 6090 |
| exons only | |
| length | 339664 |
| exons + | |
| introns | |

DRIP mRNA Structure

The DRIP gene has splice variants. The largest transcript has 6090 bp. The herein disclosed sequence contains all possible CDs. The exon sizes are reproduced in Table 2.

DRIP Transcripts

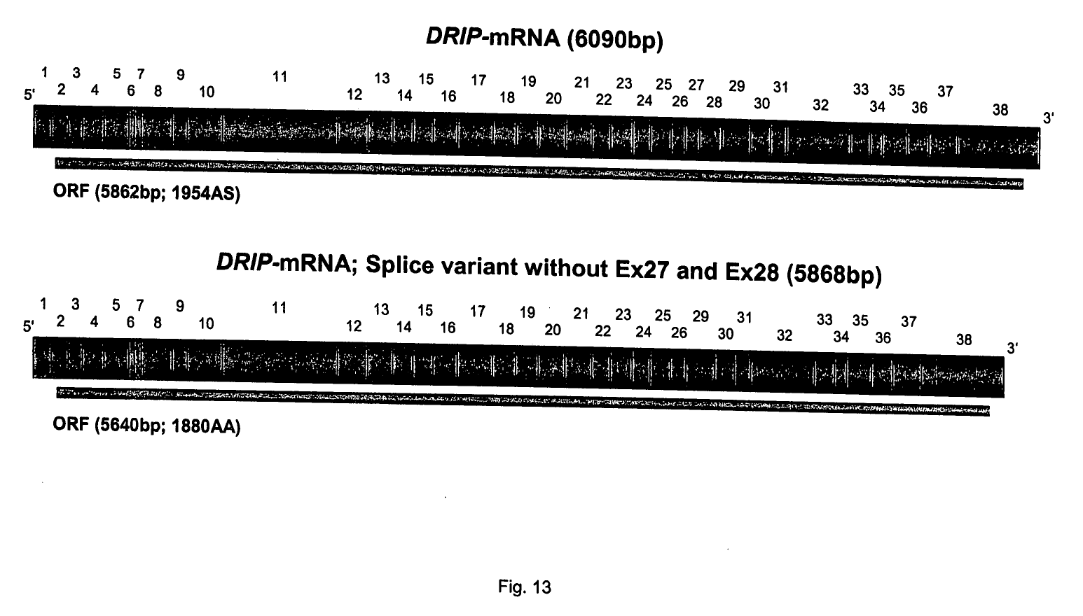

There are two DRIP transcripts. SEQ. ID. No. 11 describes the cDNA transcript of exon 1 to exon 38. SEQ. ID. No. 16 describes a splice variant of DRIP cDNA transcript of exon 1 to exon 26 and of exon 29 to exon 38. FIG. 13 is a schematic illustration of the DRIP mRNA structure.

In summary the different transcripts of the DRIP gene can be characterised as follows.

The DRIP standard transcript comprises 6090 bp and 38 exons.

In a splice variant of DRIP mRNA exon 27 and exon 28 are missing.

In addition to this, there are still DRIP-homologous EST's and mRNAs, shown in Table 4.

TABLE 3
List of homologous DRIP mRNAs, i.e. corresponding
to the gene

| | | | Date of the |
|---|---|---|---|
| | Accession no. | | data bank |
| mRNA | in gene bank | | entry in gene |
| Name | data bank | Size | bank |
| FLJ21877 | XM_087098 | 388 lbp | 06-FEB-2002 |
| FLJ21877 | NM_022065 | 2196 bp | 10-DEC-2001 |
| FLJ21877 | XM_010906 | 2125 bp | 16-OCT-2001 |
| FLJ21792 | AK025445 | 3697 bp | 29-SEP-2000 |
| death | AF323176 | 1932 bp | 20-FEB-2001 |
| receptor- | | | |
| interacting | | | |
| protein | | | |
| KIAA1767 | AB051554 | 3901 bp | 07-FEB-2001 |

DRIP Expression

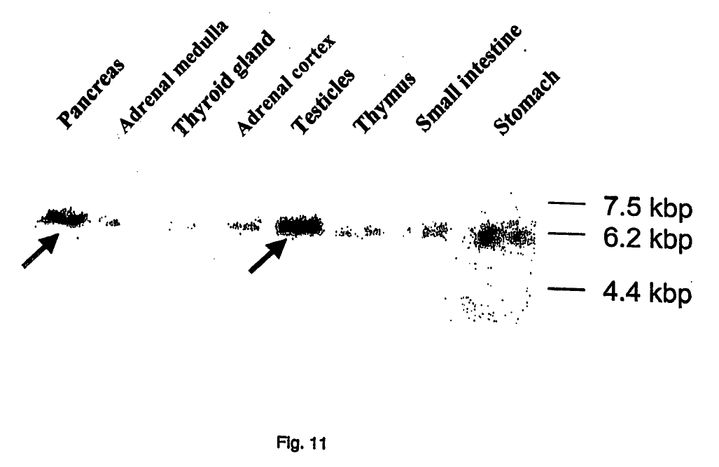

The DRIP gene is expressed ubiquitously. A reference to expression data is found in the Unigene Databank of the NCBI under cluster no. Hs 16063 Homo sapiens. This results in for example a strong expression in the Sage Northern in the tumor cell line MCF7 (Mamma tumor cell line). Evident in the Northern Blot, as shown in FIG. 11, is a reinforced expression in the stomach thyroid gland and in the testicles.

DRIP-Protein Open Reading Frame of the DRIP Gene

The open reading frame (ORF) of the DRIP gene is represented in FIG. 13 and contained in SEQ. ID. No. 11 and SEQ. ID. No. 16. The positions of start and finish of the ORF are established in the descriptions to both SEQ. ID. No.

Characterising of the Inventive Fusion Gene DRIPFUS1 from Thyroid Gland Tumors

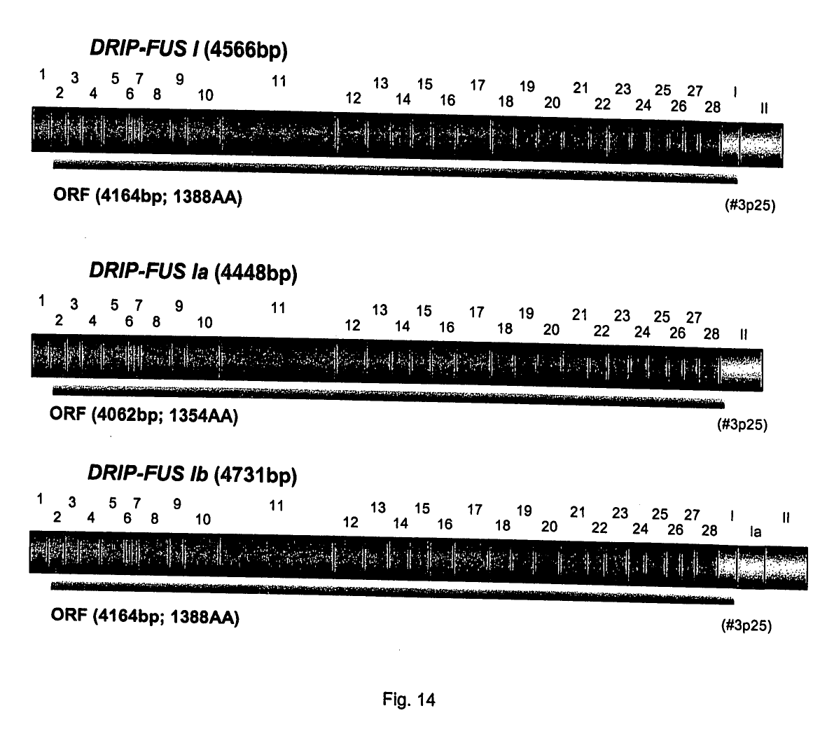

As described in greater detail in Example 1 in the thyroid gland cell line S325t/sv40 there is a fusion chromosome of (2) with a small piece of the chromosome 3 (from 3p to 3 telomer). Resulting from this fusion chromosome is a DRIP fusion protein, which is designated herein as DRIP-FUS 1. A report plan of the mRNA von DRIP-FUS I with its splice variants is represented in FIG. 14. The mRNA is shown in SEQ. ID. No. 1, SEQ. ID. No. 13, SEQ. ID. No. 14 and the amino acid sequences are shown in SEQ. ID. No 6, SEQ. ID. No. 7, SEQ. ID. No. 2.

The DRIP-FUS I fusion gene cDNA and its splice variants were determined with a standard 3′ RACE technique The added sequence of the chromosome 3 originates from the BAC clones Rpll-167M22 (Acc.: AC093174) and RplI-33519 (Acc.: AC091492). Homology of the sequence of chromosome 3 to known genes is not evident. These are new gene structures, expressed only in tumors.

Without wanting to be held to it, the present inventor assumes that the DRIP-FUSI gene is involved decisively in the hyperplasia and tumor formation. The gene or the sequence coding for it is an inventive nucleic acid and within the scope of the herein disclosed uses can be applied correspondingly. Furthermore, an inventive nucleic acid is also part of the nucleic acid sequence coding for the inventive fusion protein, originating from chromosome 2. The same applies to the part of the nucleic acid sequence originating from chromosome 3. The inventive used sequences, i.e. partial sequences, result directly from the sequence according to SEQ. ID. No. 1, SEQ. ID. No. 13, SEQ. ID. No. 14 or the annotations therefrom. The same applies for the polypeptide or the corresponding amino acid sequence.

Further fusion genes/fusion proteins are described in the figures.

Finally, it is within the scope of the present invention that all herein disclosed exons, transcripts, cDNAs, ORFs and the like understood as inventive nucleic acids and can be utilised according to the present disclosure.

Examples of the relevant tumors are in particular a first group of epithelial tumors, in particular those with a change in the chromosomal band 2p21-22, and a second group of tumors, showing change in the chromosomal Arm 2p.

Change is understood to mean herein in particular chromosome translocation, chromosome deletion, chromosome insertion and chromosome inversion.

The first group of tumors comprises in particular also tumors of the thyroid gland and preferably epithelial tumors of the thyroid gland. The second group of tumors comprises, inter alia, leukaemias, such as for example chronic lymphatic leukaemia or acute myeloic leukaemia, B-cell lymphoma, gliomas, malignant fibrous histocytomes, osteosarcomas, leiomyosarcomas, liposarcomas, ovarian tumors, breast tumors, renal carcinomas, pancreas carcinomas and gall bladder carcinomas.

According to WHO classification tumors of the thyroid gland are mostly epithelial in nature and can be divided into benign and malignant forms. With benign thyroid gland tumors a difference is made between “genuine” adenomas and thyroid gland hyperplasias, i.e. benign, adenomatous struma nodes, which are designated as “tumor-like lesions”. These hyperplasias are mostly polyclonal nodes and often have a variable, macrofollicular structure, with incomplete capsule formation. Thyroid gland adenomas on the contrary are encapsulated tumors, which are derived from the follicle epithelium. These mostly solitary occurring tumors are structured similarly and differ structurally from the neighbouring thyroid gland tissue. They can be subdivided microscopically (fine tissue) according to their degree of differentiation into normofollicular (simple/easy), macrofollicular (colloidal), microfollicular (foetal), trabecular or solid (embryonal) adenomas.

The abbreviation DRIP used herein stands for the English term “death receptor interacting protein” (German: death receptor interagierendes Protein). This protein is a ligand or adapter of death receptors (Engl. death receptors), in particular of death receptor 5(DR5), and is involved in the function of apoptosis of the cellular occurrence. The DRIP gene is confronted by a function in signal initiation or signal forwarding.

It is within the scope of the present invention that the inventive sequences generally also comprise sequences in each case complementary, which result in particular from Watson-Crick base pairing.

Within the scope of the present invention further such nucleic acid sequences are comprised, which hybridise in particular under the standard conditions for Northern Blot hybridising for the proof single-copy sequences with the sequences according to SEQ. ID. No. 1, SEQ. ID. No. 3, SEQ. ID. No. 4, SEQ. ID. No. 9 and/or SEQ. ID. No. 11 to SEQ. ID. No. 16, or would hybridise with such sequences without the degeneration of the genetic code. Suitable hybridising conditions are for example the following. A specific fragment is used as molecular probe. The radioactive marking with 32P took place according to the principle of “random primer extension”. According to a prehybridising of the inExpressHyb hybridising solution membrane (Clontech Laboratories, Palo Alto, U.S.A. ) for 30 min at 68° C. hybridising of the membrane took place with 100 ng probe inExpressHyb hybridising solution for 60 min at 68° C. Next the membrane is washed twice for 20 min at room temperature in 2×SSC/0.05% SDS and four times for 10 min at 50° C. in 0.1×SSC/0.1% SDS. The signals are detected for example on a STORM phosphorous imager (Molecular Dynamics, Sunnyvale, U.S.A.).

Nucleic acids according to the present invention are also those which without the degeneration of the genetic code would hybridise with one of the inventive nucleic acids and the nucleic acids coding for the amino acid sequence of the herein disclosed polypeptides and in particular the fusion proteins, such as for example represented as SEQ. ID. No. 1, SEQ. ID. No. 9, SEQ. ID. No. 11 and/or SEQ. ID. No. 13 to 16.

The inventive process for determining a compound, which influences the effect of a translation product of one of the inventive nucleic acids, can also be a process for screening a compound library. Such compound libraries are preferably libraries of low-molecular compounds. Basically, procedure is such that the translation product of one of the inventive nucleic acids is brought into contact with a compound in a system, whereby the system displays the effect of said translation product and thus an effect of the compound on the translation product or reflects it in the form of a detectable signal. This effect can be expressed for example in a modified function with respect to strength, substrate specificity (recognition, binding, transport) and/or receptor specificity or affinity or the membrane association, such as for example in signal forwarding. The effect could likewise have an influence on enzymatic processes inside or outside a cell. The effect can be checked both in the cell-free, in the cellular system, in the cell culture and/or in transgenic animal models, i.e. in vitro, in vivo and in situ.

As far as reference is made to tumors or hyperplasias in terms of the inventive process or applications, these are to be understood as all tumors or hyperplasias herein disclosed in connection with the inventive nucleic acids.

With the present nucleic acid sequences therefore new possibilities open up both for diagnosis and for therapy of the herein described illnesses, as well as investigation of the mechanisms associated with the occurrence in these illnesses, in particular with goitre and thyroid gland tumors or thyroid gland carcinomas. In particular there is the possibility through knowledge of the sequences to indirectly or directly use diagnostic and therapeutic means suitable on the molecular level in the sense of—pharmaceutical—compositions and drugs.

The inventive nucleic acid sequences can be used in a manner familiar to the expert in the area for realising suitable diagnostically and therapeutically usable means in the above sense as well as kits and processes.

Nucleic acids are understood to be both DNA sequences as well as RNA sequences, including hybrids thereof, including derivatives derived therefrom with altered solid such as PNA and LNA. This also includes the nucleic acids being present as single-strand, double-strand or as triple structure.

It can likewise be provided that the strandedness of the DNA, i.e. whether it is present for example single-strand or double-strand, changes over the length of the nucleic acid sequence.

In particular it can also be provided that the inventive nucleic acid sequence is present not completely, but as a fragment. Preferably the fragments are 5′-or 3′-terminal fragments of the inventive nucleic acids.

Furthermore it is within the scope of the present disclosure that the nucleic acid sequences can be present in mutated form or as alleles, as they occur in different populations. Mutation is understood herein to mean all mutations known to the specialist, which can occur inside a nucleic acid sequence, including point mutations and non-point mutations, i.e. inversions, insertions and deletions.

In particular translocations, which lead to obtaining or loss of nucleic acid sequences and/or particularly cause fusion genes.

In particular under the aspect of interaction of the inventive nucleic acid with other nucleic acids the criterion of hybridising should be employed herein, which is generally recognised in the technology. It is recognised that through choice of appropriate hybridising conditions the stringency of hybridising inside a specific region can be changed and therefore hybridising of the inventive nucleic acid sequences of nucleic acid sequences is possible, whereof the degree of deviation can fluctuate from the incomplete corresponding, i.e. complementary sequence.

Nucleic acid sequence in the inventive sense should also be understood a that sequence which would hybridise with one of the inventive sequences, if it were not the degeneration of the genetic code. This means that with a view to the open in-frame present in the inventive nucleic acid sequence, or the open in-frame, the latter can be translated into an amino acid sequence using the genetic code. As a result of degeneration of the genetic code it is possible however, based on such an amino acid sequence obtained, to again obtain a nucleic acid sequence using the genetic code, which is so different that per se it can possibly no longer hybridise with the nucleic acid sequence employed to determine the amino acid sequence.

Based on the herein disclosed nucleic acids it is possible for the specialist to generate a suitable antisense-nucleic acid, in particular antisense RNA, which can interact with the inventive nucleic acid sequences. On account of this interaction direct influencing of the processes involving the nucleic acid sequences is possible. This interaction can take place for example on the level of transcription as well as on the level of translation.

Nucleic acid sequences should herein also generally be understood as nucleic acid sequences, which can be obtained through isolating from the abovementioned tissue preferably in situ or ex vivo, for example from corresponding cell, tissue or organ cultures.

Here however corresponding nucleic acid sequences should also be understood, which can be isolated from gene banks, in particular human gene banks and still more preferably gene banks of the human chromosome 2, 3 and 7, or can be taken from the latter. In addition to this herein the term nucleic acid sequences should also comprise those nucleic acids which can be produced by means of suitable synthesis techniques, including polymerase chain reaction, and other biochemical and chemical synthesis methods known in the state of the art.

The inventive sequences can also be present in modified form.

Modification here is understood inter alia as fragmenting, insertion, deletion and reversion of (partial) sequences of the inventive nucleic acid sequences. This also includes the insertion of other nucleic acid sequences. These nucleic acid sequences can for example code for specific domains, as spacers and/or serve as elements for translation and transcription regulation.

Furthermore, the inventive nucleic acids can be modified such that they comprise sequences or molecules, which enable interaction with other molecules. This can take place for example in the form of a binding point to a solid carrier or a sequence which determines binding to a nucleic acid-binding protein.

Further still, the inventive nucleic acid sequences can be marked. Here marking should basically be understood as direct and indirect marking.

Marking can take place using the markings known in the prior art as well as marking methods and includes in particular radioactive, non-radioactive and fluorescence marking. Non-radioactive markings comprise, inter alia, the using of digoxygenin, avidin, streptavidin and biotin.

Vector should be understood herein in particular as recombinant vectors, as they are known in the technology.

The term of vector as used herein includes, inter alia, viral vectors, such as for example adenoviral or retroviral vectors and phage systems, as well as plasmide vectors, including cosmide vectors, and artificial chromosomes, which can be used in prokaryontic and/or eukaryontic systems.

Apart from the inventive nucleic acid sequences the inventive vectors may comprise ongoing elements, known in the prior art. The respective elements, such as for example promoters, terminators and enhancers, are selected according to the respective host cell system in a manner known to the specialist. In particular the use of a suitable eukaryontic promoter and of an inducible promoter is preferred. Aside from the use of the abovementioned elements singly or in any combination it is also still within the scope of the present invention for such elements to be contained in this type of vectors, which result in at least the inventive nucleic acid sequence, or a part thereof, becoming integrated into the genome of the host cell system.

It is also within the scope of the present invention that at least one of the abovementioned elements in the in-frame (“in-frame”) is connected to at least one open in-frame of the inventive nucleic acid sequences, and it is quite particularly advantageous if the transcription rate of the special open in-frame is controlled by means of an additionally introduced promoter, whereby the promoter is then arranged typically at a suitable distance and “in-frame” to the open in-frame.

Thereby it can also be provided that an open in-frame of the inventive nucleic acid sequences, preferably under the abovementioned conditions, has a signal sequence which enables translocation of the gene product coded by the open in-frame via a membrane, and if required as a result of this enables further modification of the gene product. Such signal sequences include those for transport of the synthesised protein to the endoplasmatic reticulum, Golgi apparatus, to lysosomes, to cell organelles, such as mitochondria and chloroplasts, as well as to the cell core. Such possible traversing of different cellular compartments allows posttranslational modification and thus if required ongoing beneficial forming of the gene product.

Apart from signal sequences such a construct may also contain additional nucleic acid sequences, which result in the gene product of an open in-frame of the inventive nucleic acid sequences forming a fusion protein, whereby the fused part of a domain can correspond to another protein and e.g. serves to detect the gene product of the open in-frame of the inventive sequences, or the interaction with other molecules or structures in the biological system, whereby the biological system is preferably the cell.

Apart from the abovementioned and the further advantages originating for the specialist from the description of the inventive nucleic acid sequences these are also inherent to the polypeptides which can be derived from the abovementioned nucleic acid sequences or coded by the latter. These polypeptides can be derived for one directly from an open in-frame of the inventive nucleic acid sequence, or can be produced by an inventive vector expressed in a host organism in a manner familiar to the specialist. In the process the host organism is first typically transformed with the inventive vector, the host organism is augmented and the polypeptide is obtained from the host organism, or in the case of secretion of same in the medium is obtained from the latter.

Apart from direct use of the inventive polypeptides and of the nucleic acids coding them for influencing the—cellular—events in thyroid gland tissue the latter can also be used in cleaning, for example via affinity chromatography, of other components involved in cellular events, or for manufacture of suitable antibodies, which, inter alia, for their part can then be employed for therapeutic and/or diagnostic purposes. In this way inter alia interaction partners of the inventive polypeptides can therefore be detected or isolated.

Depending on the existing requirements in each case, such as posttranslational modification or desired degree of purity and the like, either a prokaryontic or an eukaryontic host organism can be of advantage in the manufacture of the inventive polypeptides in a host organism.

The inventive polypeptides can be modified in a suitable manner. Modification should be understood, inter alia, as fragmenting, in particular shortening, of the molecule.

Modification in this sense also includes marking of the polypeptide. The latter can take place by means of both high-molecular and low-molecular compounds and includes radioactive, non-radioactive and fluorescence marking.

Marking can also be present for example in the form of phosphorylising or glycosylising of the protein.

Corresponding marking methods or modification methods are known in the technology (see e.g. Protein Methods, 2nd ed., D. M. Bollag et al., Wiley Liss, Inc., New York, N.Y., 1996) and are recorded herein in full by reference.

Modification is understood according to the present invention to also mean any form of posttranslational modification, as known to experts, in particular proteolytic processing of the polypeptide, attaching or separating of radicals at the N-terminus, acetylising of the N-terminus, myristoylising of the N-terminus, attaching of glycosyl-phosphatidyl radicals, farnesyl radicals or geranylgeranyl radicals to the C-terminus, N-glycosylising, O-glycosylising, attaching of glycosaminoglycan, hydroxylising, phosphorylising, ADP ribosylising and formation of disulfide bridges.

It is also within the scope of the present invention that the inventive polypeptides come from or are derived from mutation(s), in particular amino acid mutation(s) from an inventive polypeptide. Amino acid mutation is understood both as mutation, in which an amino acid is exchanged for an amino acid similar in side chain (conservative mutation), for example I to L or D to E, as well as mutation, in which one amino acid is exchanged for another amino acid, without this exchange having a disadvantageous effect on the function of the coded polypeptide (still mutation). The function of the coded polypeptide can be checked by a suitable assay. Suitable functional assays for DRIP, i.e. for apoptotic procedures, are known to specialists in the field (see e.g. Lizard G. al., Ann Pathol 1997 Mar; 17 (1): 61-6.).

Inventive cells are used to bring about substantial advantages. These comprise inter alia the manufacture of corresponding nucleic acid sequences or gene products derived therefrom.

In particular the insertion of the inventive nucleic acid sequences in the genome of a cell is of particular significance, for example for further study of the influence of such sequences, in particular in the cellular environment. At the same time gene dose effects and the like can be examined or utilised for diagnostic and/or therapeutic purposes. A state, in which based on diploid cells only one chromosome contains one or more of the inventive nucleic acid sequences, and preferably inserted in the position corresponding to their position in band 2p21-22, and the second chromosome has no chromosome translocation including the chromosomal band 2p21-22, seems quite particularly advantageous. Such cells can serve for example as positive and/or negative control in a diagnostic preparation.

It is within the scope of the present invention that apart from the respective inventive translation product or the nucleic acid(s) coding therefor, as described herein, other means can also be employed to generate or also to suppress the effects originating from the respective translation product or the nucleic acid coding therefor. Such means can be detected for example within the scope of a so-called screening process. In a first step one or more so-called candidate compounds is prepared. Candidate compounds in the sense of the present invention are those compounds whereof the suitability should be established in a test system for treating the herein disclosed illnesses, in particular functional disturbances of the thyroid gland and/or hyperplasias of the thyroid gland and/or thyroid gland tumor, or should be employed as diagnostic means for the diagnosis of same. If the candidate compound in the described test system displays a corresponding effect, it represents a corresponding, i.e. in principle suitable means for treatment of these illnesses. In a second step the candidate compound is brought into contact with a (translation product) expression system or with a (translation product) activity system. A translation product-expression system is thus an expression system, which shows the expression of the translation product, whereby the extent of the expression can be fundamentally changed. A translation product activation system is essentially an expression system, whereby less is placed on the expression of the translation product than on its activity or its activation status and its capacity for influence. It should be noted here that the translation product as such does not have to be the result of an actual expression procedure, but also can be added to the corresponding activity system already as a polypeptide or protein. In this case it is established in concrete terms whether the activity of the translation product alters under the influence of a candidate compound. At the same time, independently of the presence of the respective concrete expression system or activity system, basically both a reduction in the expression or activity and also an increase in the expression or activity take place.

Typically the expression system and/or the activity system is an in vitro preparation, such as for example a cell extract or a fragment of a cell extract such as for example a cell core extract. But such a preparation can also be a (bio)chemical preparation more or less precisely defined with respect to the relevant component. A translation product expression system in terms of the present invention can however also be cell, preferably a cell of the thyroid gland, of hyperplasias of the thyroid gland or of the cells forming a thyroid gland tumor.

An observation to what extent an increase or decrease of the expression system takes place can be established on one level of the expression, i.e. for example by increasing or decreasing the quantity of the nucleic acid coding for the translation product, in particular the mRNA, or also of the translation product produced in the expression system under the influence of the candidate compound, i.e. of the respective protein. The required techniques such as for example a process for quantifying mRNA are known to the specialist in the field and for example described in Sambrook et al. (Sambrook, Joseph: Molecular Cloning: A laboratory manual/J. Sambrook; E. F. Fritsch; T. Maniatis Cold Spring Harbor, NY: Cold Spring Harbor Laboratory Press),19XX1. ed. T. Maniatis, Thomas P.: Molecular Cloning; 3rd edition: Joseph Sambrook and David W. Russell; Literary references ISBN 0-87969-309-6 ISBN 0-87969-576-5 ISBN 0-87969-577-3 1.-2. ed., 4. [Dr.]. -1989). Moreover, the specialist is also familiar with a process for determining the content of the quantity of translation product, for example through use of suitable antibodies.

Antibodies can be manufactured according to the generally known process described for example in Current Protocols (“Current Protocols in Protein Science”, published by Coligan J. E.; Dunn B. M., Hidde L. P. Speicher D. W., Wingfield P. T.; John Wiley & Sons). It is also possible that within the scope of the expression system the resulting translation product bears marking. Such suitable marking is for example marking with His6 (Janknecht R et al. (1991) Proc Natl Acad Sci USA 88 (20): 8972-6).

In the case of the translation product activity system the increase in activity or decrease in activity of the translation product is typically tested in a functional assay, as has been described already in context with the definition of the mutated translation products, or more precisely the mutated inventive polypeptides.

Placing candidate compounds and expression system or activity system in contact takes place as a rule by addition of a preferably aqueous solution of the candidate compound to the corresponding reaction system, i.e. the expression system or the activity system, which are also designated herein generally as test systems. The aqueous solution can preferably be a buffer solution.

The use of candidate compounds occurs as a rule such that in each test in each case only a single candidate compound is used in the corresponding test system. At the same time it is also within the scope of the present invention that corresponding tests are performed parallel and in a high throughput process.

The last step of the inventive screening process, consisting of determining whether the expression or activity of the translation product or a nucleic acid coding therefor is altered under the influence of the candidate compound, as a rule takes place by comparing the behaviour of the test system without addition of the candidate compound to that of the test system by addition of the candidate compound. Preferably, the candidate compound is contained in a compound library. Every compound library is basically conceivable independent of the compound class as a compound library. Suitable compound libraries are for example libraries of small molecules. It is however also within the scope of the present invention that other compound classes are used as small molecules, such as for example peptides, proteins, antibodies, anticalins and nucleic acids, in particular also functional nucleic acids.

At the same time it is within the scope of the present invention that the inventive translation product or the nucleic acid coding therefor can be sued as target molecules for generating the abovementioned compound classes such as in particular peptides, proteins, antibodies, anticalins and functional nucleic acids. The corresponding compounds, i.e. peptides, proteins, antibodies, anticalins and functional nucleic acids, can then be utilised in the inventive screening process.

It is further within the scope of the present invention that those compounds, i.e. peptides, proteins, antibodies, anticalins and functional nucleic acids, which are generated against the inventive translation product or the nucleic acid(s) coding therefor, are used as means in the sense of the present invention, i.e. as means for the therapy of functional disturbances of the thyroid gland and/or hyperplasias of the thyroid gland and/or thyroid gland tumors or as corresponding diagnostic agents.

Peptides and in particular binding peptides in the sense of the present invention are preferably those proteins and peptides, which bind to the inventive translation product or a or the interaction partner, in particular the natural interaction partner, of the inventive translation product, preferably in biological systems, in particular in the thyroid gland or hyperplasias of the thyroid gland or thyroid gland tumors and the cells forming them. The same applies also for the antibodies, anticalins, functional nucleic acids and small molecules described herein in connection with the inventive use. At the same time each member of the abovementioned compound classes can preferably interact with the inventive translation product or a nucleic acid coding therefor and therefore influence the corresponding activity of the translation product or the coding nucleic acid. The conditions, under which such interacting takes place, are familiar to specialists and correspond to those conditions under which interacting should occur in practice. Preferably, the conditions are physiological conditions, preferably conditions prevalent in a biological system of the illnesses described herein.

It is however also within the scope of the present invention to create or to use such members or undergo a corresponding screening process, which with the interaction partner, in particular the natural interaction partner of the inventive translation product, or a nucleic acid coding therefor, interact and influence the expression or activity of the interaction partner or the nucleic acid coding therefor. Here, too, the term “influence” designates either an increase or decrease in expression, of the extent of expression or the activity, as generally disclosed herein.

As a result of the interaction of said members with the interaction partners of the inventive translation product or the nucleic acid coding therefor therefore the cascade of reactions coupled with the translation product is interrupted, so that the naturally observed effect of the inventive translation product or of the nucleic acid coded thereby is interrupted, which can be used within the scope of the herein disclosed therapy or diagnosis concerning functional disturbances of the thyroid gland and/or hyperplasias of the thyroid gland and/or thyroid gland tumors.

These peptides, hereinbelow also designated as binding peptides or binding proteins, can be screened or manufactured using a process known form the prior art, such as for example phage display. The techniques are known to specialists in the field. At the same time the production of such peptides typically proceeds such that a peptide library is laid out, for example in the form of phages, and this library is put in contact with a target molecule, in the present case for example with a translation product or the natural interaction partner of the translation product of the inventive nucleic acids. The binding peptides are then typically removed as complex along with the target molecule from the non-binding members of the library. It is within the scope of the knowledge of specialists in this area that the binding properties depend at least to a certain extent on the trial conditions present in each case, such as for example salt content and the like. After separation of the peptides with a higher or stronger affinity binding to the target molecule from the non-binding members of the library or the target molecule, in the present case for example the inventive translation product, the latter can then be characterised. If required, prior to characterising an amplification step is necessary, for example by increasing the coding phages corresponding to the peptide or the peptides or protein(s). The characterising comprises preferably sequencing the proteins binding on the translation product or its natural interaction partner, depending on which of both molecules was used as target molecule in the phage-display screening process. The peptides are basically not limited with respect to their length. Typically, peptides with a length of 8-20 amino acids are obtained or used in such processes, however. The size of the libraries is 10²-10¹⁸, preferably 10⁸-10¹⁵ different peptides. A special form of peptides binding on target molecules is represented by anticalins, such as described for example in the German patent application DE 197 42 706.

In the light of the herein disclosed technical idea antibodies can then also be produced against a gene product, in particular an inventive translation product such as a polypeptide, or the inventive nucleic acid sequences. In this case also therefore the advantages familiar to the specialist generally result from the presence of antibodies against chemical compounds, in particular in in vitro and in vivo applications. In particular, with the antibodies for example purifying of the inventive compounds, i.e. the inventive nucleic acids and/or the inventive polypeptides or translation products is possible, whereof detection, but also influencing of the biological activity, including biological availability, of the compounds, is directed at that of the antibodies, both in situ, and ex vivo, in vivo and/or in vitro. In more precise terms, the gene products can be specifically detected in particular using monoclonal antibodies, or on the cellular level interaction of the gene products. or nucleic acid sequence with other cellular components can be influenced and therefore specifically intervene in the cellular events. Depending on the effect of the respective compounds, at which the antibody is directed and at which it develops its effect in the observed system, stimulating and inhibiting effects can be achieved in principle.

Antibodies are understood herein both as polyclonal antibodies and as monoclonal antibodies. However, monoclonal antibodies are particularly preferable on account of increased specificity. However, there are those cases where the purity or specificity of polyclonal antibodies is adequate or the plurality of specificities realised in polyclonal antibodies and other properties can be used to advantage.

The manufacture and use von antibodies is described for example in Antibodies: A Laboratory Manual (E. Harlow & D. Lane, Cold Spring Harbor Laboratory, NY, 1988), of which the disclosure is recorded herein by reference.

It is also within the scope of the invention that the antibody can also be a single-chain antibody.

It is also within the scope of the present invention that the antibody is fragmented, in particular shortened. This includes the antibody being referenced to a major extent, as long as there is the antibody-specific property, i.e. binding to a defined epitope. Referencing is particularly of advantage when the corresponding antibody is to be employed at the cellular level, since it has improved permeations and diffusion properties relative to a complete antibody.

In addition still other forms of the modification are provided, which are known to the specialist in the field and are described for example in Antibodies: A Laboratory Manual (E. Harlow & D. Lane, Cold Spring Harbor Laboratory, NY, 1988). In general it can be affirmed that the modifying of antibodies can occur in principle similarly to how that of polypeptides and, to a specific extent, nucleic acids, and the abovementioned on this subject also applies for the inventive antibody.

A further compound class, which can be used within the scope of the inventive screening process as also with the inventive manufacture of drugs and diagnostic agents, is functional nucleic acids. Functional nucleic acids should be understood herein in particular as aptamers, aptazymes, ribozymes, spiegelmers, antisense oligonucleotides and RNAi.

Aptamers are D-nucleic acids, either single-strand or double-strand, based on RNA or DNA, which bind specifically on a target molecule. The manufacture of aptamers is described for instance in European Patent EP 0 533 838. The procedure is as follows:

A mixture of nucleic acids, i.e. potential aptamers, is prepared, whereby each nucleic acid comprises a segment of at least eight successive, randomised nucleotides and this mixture is brought into contact with the target, in the present case therefore with a translation product, in particular an inventive translation product, nucleic acid(s) coding therefor, interaction partners of the translation product, in particular natural interaction partners, and/or nucleic acid(s) coding for the latter, whereby nucleic acids, which bind on the target, are separated from the rest of the candidate mixture if required on the basis of increased affinity, are compared to the affinity of the candidate mixture, and the resulting nucleic acids binding on the target are amplified. These steps are repeated a number of times, so that by the end of the process nucleic acids binding specifically on the respective target or target molecule, even the so-called aptamers, are obtained. It is within the scope of the present invention that these aptamers can be stabilised, for instance by introducing specific chemical groups, which are known to specialists in the field of aptamer development. Aptamers are currently already being used therapeutically. It is also within the scope of the present invention that the resulting aptamers are used for target validating and as messenger substances for the development of drugs, in particular of small molecules.

The manufacture or production of spiegelmers is based on a fundamentally similar principle, which can be developed as target molecule within the scope of the present invention on the basis of an inventive translation product, the nucleic acid(s) coding therefor, the translation product interaction partners, in particular natural interaction partners, and/or the nucleic acid(s) coding for the latter.

The manufacture of spiegelmers is described for instance in international patent application Wo 98/08856. spiegelmers are L-nucleic acids, i.e. they comprise L-nucleotides, and are characterised substantially in that in biological systems they have very high stability and at the same time, comparable to aptamers, specifically interact with a target molecule or can bind on the latter. With the manufacture of spiegelmers the procedure is such that a heterogeneous population of D-nucleic acids is generated, which population is brought into contact with the optical antipodes of the target molecules, in the present case therefore for example with the D-enantiomer of the naturally occurring L-enantiomer of a translation product, then those D-nucleic acids are separated out which do not interact with the optical antipodes of the target molecules, the D-nucleic acids, interacting with the optical antipode of the target molecules, are determined, if required separated out and sequenced and then L-nucleic acids are synthesised, which are identical in their sequence with those sequence(s) previously detected for the D-nucleic acid(s). Similar to the process for manufacture of aptamers it is also possible in this case to enrich or produce suitable nucleic acids, i.e. spiegelmers, by repeating the steps, which bind the translation product, one or more of its especially natural interaction partners, depending on which of the abovementioned compounds is used as target molecule, or a nucleic acid coding therefor.

Another form of the functional nucleic acids, which can be used according to the present invention, are so-called aptazymes. Aptazymes are described for example by Piganeau, N. et al. (2000), Angew. [Applied] Chem. Int. Ed., 39, no. 29, pages 4369-4373. At the same time these are a specific form of aptamers, which are characterised in that apart from the aptamer portion, which binds specifically on the target molecule, in the present case an inventive translation product or an interaction partner thereof, they can still contain a ribozyme portion with the result that after binding of the target molecules of the aptamer portion the aptazyme of the ribozyme portion is activated and this results in splitting of a nucleic acid functioning as substrate of the ribozyme portion of the aptazyme. With corresponding configuration of the ribozyme substrate a change in the fluorescence as a result of a change in the spatial arrangement of a fluorescence donor relative to a fluorescence acceptor can be observed for example on the ribozyme substrate. In this respect aptazymes are suited in particular for application within the scope of target validation concerning a translation product and its interaction partner as well as diagnostics agents in the sense of the present invention. In similar fashion is also therapeutic use is possible to the extent that it is disclosed in connection with the herein described ribozymes.

Common to the abovementioned compound classes is the fact that they preferably bind on the respective protein or polypeptide, i.e. the or a, preferably an inventive, translation product or the interaction partner thereof, or are generated against the latter. It is however also within the scope of the present invention that these compound classes, and in particular the functional nucleic acids, have as target molecule the nucleic acid(s) coding for the abovementioned preferably inventive proteins or polypeptides or the inventive nucleic acids as target molecule.

An additional class of compounds, which can be produced or developed using a translation product, preferably an inventive translation product, and/or of an interaction partner thereof and in particular the nucleic acid(s) coding for these or an inventive nucleic acid, are ribozymes, antisense oligonucleotides and RNAi.

Common to all these classes of compounds is the fact that they display their effect not on the level of the translation product, i.e. on the level of the proteins (translation product and interaction partner thereof), but on the level of the nucleic acid(s) coding for the respective protein, in particular the mRNA or the mRNA coding for a translation product, which codes for an interaction partner of the translation product. The corresponding genomic DNA or the corresponding cDNA is also suited as target molecule.

Ribozymes are catalytically active nucleic acids which are preferably constructed from RNA and comprise two partial areas. The first partial area is responsible for catalytic activity, whereas the second part is responsible for specific interaction with a target nucleic acid. When it comes to forming an interaction between the target nucleic acid and the second part of the ribozyme, typically by hybridising of base areas substantially complementary to one another, the catalytic part of the ribozyme can hydrolyse the target nucleic acid either intramolecular or intermolecular, whereby the latter preferably is, in the event that the catalytic effect of the ribozyme is a phosphodesterase activity. As a result there is, if required, further decomposition of the coding nucleic acid, whereby the titre of the target molecule both on the nucleic acid and also the protein level and in intracellular and extracellular fashion can be reduced and therefore a therapeutic preparation for the treatment of functional disturbances of the thyroid gland and/or hyperplasias of the thyroid gland and/or thyroid gland tumors is provided. Ribozymes, whereof the use and construction principles are known to the specialists in the field, are described for instance in Doherty and Doudna (Ribozyme structures and mechanisms. Annu. Rev. Biophys. Biomol. Struct. 2001; 30: 457-75) and Lewin and Hauswirth (Ribozyme gene therapy: applications for molecular medicine. Trends Mol. Med. 2001, 7: 221-8). With respect to a pharmaceutical composition, which comprises a ribozyme in addition to the pharmaceutically acceptable carrier or the use of the inventive ribozyme as drug, and in particular for treatment of functional disturbances, hyperplasias and tumors of the thyroid gland, it is also possible that the ribozyme is constructed such that it works specifically on one or more of the inventive nucleic acid sequences and therefore also controls the expression or translation at a cellular level, which is of significance in particular in the therapeutic and also the diagnostic aspect. At the same time and on account of the fact that ribozymes display intramolecular and intermolecular catalytic effects, it can also be provided that the inventive nucleic acid sequences are modified such that areas are split off by ribozyme activity of the changed area. Also there is particularly the therapeutic possibility which is possible for the specialist to realise in light of the now available sequence information.

Ribozymes, similar to the inventive nucleic acid sequences themselves, are particularly advantageous, though not only when they are introduced to the effector cell by means of gene therapy for example. However it is also conceivable that corresponding modifications are made ex vivo and such modified cells are then available for re-implantation, either allogenic or autogenic. The manufacture and use of ribozymes is disclosed in Ribozyme Protocols (Philip C. Turner, Ed., Humana Press, Totowa, N.Y., 1997) and is included herein. This applies basically for each of the herein described functional nucleic acids.

The use of antisense oligonucleotides for manufacture of a drug or diagnostic agents in the sense of the present invention is based on a basically similar work mechanism.

Antisense oligonucleotides hybridise as a result of base complementarity typically with target RNA, normally with mRNA and thereby activate RNaseH. RNaseH is activated both by Phosphodiester as well as phosphorothioate-coupled DNA. Phosphorodiester-coupled DNA is quickly broken down however with the exception of phosphorothioate-coupled DNA by cellular nucleases. These resistant, non-naturally occurring DNA derivatives do not inhibit RNaseH when they are hybridised with RNA. In other words, antisense polynucleotides are effective only as DNA-RNA hybrid complex. Examples for such antisense oligonucleotides are found inter alia in U.S. Patent U.S. Pat. No. 5,849,902 or U.S. Pat. No. 5,989,912. In principle the essential concept of the antisense oligonucleotides comprises providing a complementary nucleic acid against certain RNA. In other words, based on the knowledge of the nucleic acid sequence of a translation product or its interaction partner(s), in particular the respective mRNA, suitable antisense-oligonucleotides can be produced by base complementarity, which result in a breakdown in nucleic acid, preferably coding nucleic acid such as hnRNA or mRNA. This breakdown can then be detected in an expression or activity system qualitatively or quantitatively.

A further class of compounds, which can be identified, produced or used as drug or diagnostics means in the sense of the present invention, is the abovementioned RNAi. RNAi is a double-strand RNA, which permits RNA interference and typically has a length of approximately 21 to 23 nucleotides. At the same time one of the two strands corresponds to the RNA of a sequence of a or of the gene to be broken down or the corresponding mRNA. In other words, based on the knowledge of the nucleic acid(s) coding for a translation product and/or its interaction partner, in particular mRNA, a double-stranded RNA can be produced, whereby one of the two RNA strands is substantially complementary to said nucleic acid(s) coding for the translation product and/or its interaction partner, preferably mRNA, and this then leads to breakdown of the corresponding coding nucleic acid and thus to a reduction of the titre of the respective translation products, i.e. proteins. The production and use of RNAi as drug or diagnostic means is known to specialists in the field and described for instance in international patent applications WO 00/44895 and WO 01/75164.

With respect to the effective mechanisms of the abovedescribed classes of compounds, namely ribozymes, antisense oligonucleotides and RNAi, it is therefore within the scope of the present invention, apart from the inventive translation product, i.e. the respective polypeptide or protein, and in particular its natural interaction partner(s), to use either directly or as target molecule, and either fully or partially the nucleic acid(s) in each case coding therefor, in particular the corresponding RNA, cDNA or genomic DNA, for manufacture of a drug for treatment of functional disturbances of the thyroid gland and/or hyperplasias of the thyroid gland and/or thyroid gland tumors or for the manufacture of diagnostic means for diagnosis of the abovementioned illnesses or functional disturbances and for monitoring the course of the illness or the applied therapy.

With use of the inventive translation product, the nucleic acid coding therefor, of the translation product interaction partner(s), in particular the natural interaction partner(s), and/or the nucleic acid(s) coding for these as target molecule for the manufacture or development of a drug for treatment or therapy of functional disturbances of the thyroid gland and/or hyperplasias of the thyroid gland and/or thyroid gland tumors, as also for the manufacture and/or development of agents for diagnosis of same, libraries of small molecules can also be used. Here too the target molecule is put in contact with a library and those members of the library, which bind thereon, are separated if required from the other members of the library or the target molecule and optionally characterised further. Here too the characterising of the small molecules takes place according to processes known to the specialists in this area, so e.g. the compound is identified and the molecular structure is determined. The libraries comprise as few as two and as many as up to several hundred thousand members.

In connection with the different herein disclosed classes of compounds, which can be used as therapeutic means or diagnostic means according to the present invention, it is an aspect that certain classes bind directly with a translation product or its interaction partner interact or on the latter. It is however also within the scope of the present invention that said compounds of the different classes, in particular when these are peptides, antibodies, anticalins, aptamers, aptazymes and spiegelmers, block the interaction partner of the translation product by one more or less specific interaction for the translation product.

In this respect the term of use of a translation product herein is also to be understood to the extent that it comprises the use of one or more of the translation product interaction partner(s), such as for instance receptors and transcription and translation factors. The herein described processes are then to be modified to the extent that instead of the translation product protein or the nucleic acid coding for them, an interaction partner thereof is used in the different selection methods, assays, screening processes or manufacturing methods.

Within the scope of the present invention interaction partners of a translation product, in particular of an inventive translation product, in particular natural interaction partners, are understood as those molecules and structures, with which said translation product interacts. At the same time in particular those molecules and structures are comprised, with which the protein interacts in a biological system under normal and/or also under pathological conditions. A suitable interaction partner of DRIP is for example DR5 (Engl. death receptor 5), such as is described for example in Hymowitz S. G., Christinger H. W., Fuh G., Ultsch M., O'Connell M., Kelley R. F., Ashkenazi A., de Vos A. M., OMIM, Protein, Structure Triggering cell death: the crystal structure of Apo2L/TRAIL in a complex with death receptors. Mol Cell. 1999 Oct; 4 (4): 563-71. Ashkenazi A, Dixit V M. Related Articles, Nucleotide, Protein Apoptosis control by death and decoy receptors. Curr. Opin. Cell Biol. 1999 April 1 (2) :255-60. Review, or Mitsiades N, Poulaki V, Mitsiades C. S., Koutras D. A., Chrousos G. P. Related Articles, Apoptosis induced by FasL and TRAIL/Apo2L in the pathogenesis of thyroid diseases. Trends Endocrinol. Metab. 2001 Nov; 12 (9) :384-90. Review.

With respect to makeup of the inventive kits for diagnosis and/or therapy of functional disturbances, hyperplasia and tumors of the thyroid gland, i.e. generally the herein described illnesses, it is to be noted that the precise configuration of such a kit, i.e. which components are included explicitly, is known to the specialist in this area, and in particular is possible in light of the above embodiments with respect to the effect or applicability of the herein disclosed inventive nucleic acid sequences and their translation product. The inventive kit is in any case comprised of at least one of the inventive elements, which is selected from the group, which comprises the inventive nucleic acid(s), the vector, the polypeptide, the cell, the antibody, the ribozyme and at least one of the different compounds of the different classes, as were characterised herein, in particular the antibodies, peptides, proteins, anticalins, small molecules, aptamers, spiegelmers, ribozymes, aptazymes, antisense oligonucleotides and RNAi, specifically for the inventive nucleic acids or inventive translation products and the interaction partners thereof, in particular the natural interaction partners, and the nucleic acid in each case coding therefor, in each case preferably in their inventive shaping. Further of the inventive kits, which can be contained individually or in combination in the inventive kit, are selected from the group, comprising buffer, negative controls, positive controls and instructions for use.

Typically the individual compounds or constituents are contained in the kit in dry or liquid form, preferably portioned for the individual case. The kit can preferably be used for determining functional disturbances of the thyroid gland and/or hyperplasias of the thyroid gland and/or thyroid gland tumors. With respect to the use of inventive cells their use is comprised in particular as reimplant or as positive and/or negative control in corresponding diagnostic or therapeutic preparations within the scope of the present invention.

With the inventive process for proving functional disturbances and hyperplasias and/or tumors of the thyroid gland, also in the sense of carcinomas and goitre, it is possible that the thyroid gland material is present in situ, ex vivo, in vivo or in vitro. The term “thyroid gland material”, as used herein, designates in particular material, preferably cellular material of the thyroid gland in its normal and/or pathogenic state. The term thyroid gland material thus also comprises material of thyroid glands with a functional disturbance, of hyperplasias of the thyroid gland, of tumors of the thyroid gland, including carcinomas and goitre. Determining whether a functional disturbance, hyperplasia and/or tumor is present takes place by comparing the effect of the used inventive agent or agents on the thyroid gland material to be examined with its effect on “normal” thyroid gland tissue. Further exemplary embodiments of the inventive process will emerge for the specialist on the basis of the herein contained disclosure.

The above embodiments with respect to the different embodiments and the thus realisable advantages also apply in essence for the inventive drugs, and in particular for those means for diagnosis and/or therapy and/or prevention of functional disturbances, hyperplasias and/or tumors of the thyroid gland in the form of the inventive sequence(s), or their use, in the form of the inventive vector, or its use, in the form of the inventive polypeptide, or its use, in the form of the inventive cell, or its use, in the form of the ribozyme, or its use, disclosed herein. The same applies also for the compounds manufactured or selected according to the present invention from the group comprising binding peptides, anticalins and functional nucleic acids, whereby it is common to these compounds that they are directed against the inventive nucleic acid sequences or the polypeptides coded thereby or their interaction partner(s) or the nucleic acid coding therefor, i.e. against these are manufactured or selected as target molecules. The corresponding drugs can have a content of one or more of the abovementioned agents or compounds.

The same applies also for the inventive pharmaceutical compositions. At the same time it is possible that apart from the optional pharmaceutically acceptable carrier a pharmaceutical composition comprises only one of the listed compounds. Alternatively, it is also within the scope of the invention that the pharmaceutical composition has several of the abovementioned compounds apart from the pharmaceutically acceptable carrier.

Pharmaceutically acceptable carriers are to be understood herein as all carriers known to the specialist, which typically can be selected depending on the form of application. A pharmaceutically acceptable carrier can, inter alia, be water, buffer solutions, alcoholic solutions and the like.

With respect to the inventive processes for manufacturing primers, it is pointed out that these are those which allow specific depiction of the inventive nucleic acid sequences, or parts thereof.

In the inventive process for displaying an inventive nucleic acid sequence the inventive nucleic acid sequences or sequences corresponding thereto or their complementary sequences using inventive primers as primer for a polymerase chain reaction can be used in all their different designs, from whichever source. The design of suitable primers and the carrying out of polymerase chain reactions is disclosed for example in PCR & PCR 2: A practical approach (M. J. McPherson, P. Quirke and G. R. Taylor, Eds., IRL Press, Oxford, England 1991, and M. J. McPherson and B. D. Hames, Eds., IRL Press, Oxford, England 1995) and is included herein.

The inventive nucleic acids, including inventive nucleic acid sequences, comprise in particular the herein disclosed DRIP gene, which is designated herein also as DRIP in special configurations of the inventive DRIP and the nucleic acids coding for the inventive fusion proteins.

Nucleic acids or nucleic acid sequences according to the present invention are in particular also the following nucleic acids, which comprise the nucleic acid sequences designated herein with “SEQ. ID. No.”, those nucleic acids or nucleic acid sequences, which partially or fully comprise the nucleic acid sequences designated hereinbelow with “SEQ. ID. No.”, and comprise further nucleotides, in particular on the 5′- and/or 3′ end, those nucleic acids, which contain further nucleotides in addition to the sequences designated herein with “SEQ. ID. No.”, in the case of the inventive DRIP sequence or gene supplemented by further nucleotides from the BAC RP11 339H12 or RP11 204D19. The relative arrangement of the abovementioned clone is illustrated in FIG. 12.

It is also within the scope of the present invention that parts or fragments of the inventive nucleic acids as such are designated and/or used as inventive nucleic acids. This applies in particular to the use of the inventive nucleic acids for manufacture or generating drugs on the basis of RNAi, also designated as siRNA, ribozymes, aptazymes and antisense molecules. This is fundamental in the construction principle of the above compound classes, which are substantially identical or complementary to the inventive nucleic acid, in particular to the sequences specified in the sequence protocol. The length of the area substantially identical or complementary to the inventive nucleic acids is preferably 15 to 49, preferably 15 to 31 and most preferably 21 to 23 nucleotides. Nucleic acids according to the present invention are thus also those parts or fragments of the inventive nucleic acids, in particular also of the sequences specified or contained in the sequence protocol, which comprise 15 to 49, preferably 15 to 30 and most preferably 21 to 23 nucleotides. The starting point for such a sequence or such a nucleic acid molecule can be at any point on the respective nucleic acid or nucleic acid sequence, preferably at a point where the number of nucleotides, which are still present to the very end of the sequence, suffices to provide the nucleotides required for the desired length area.

As used herein, designated a size order range from X to Y all lengths X, Y and all whole-number lengths in between.

So comprises for example the length area 21 to 23 nucleotides die lengths von 21, 22 and 23 nucleotides.

Furthermore, inventive nucleic acids are also the herein disclosed primers and in particular those other fragments, which are individualised or can be individualised with respect to their sequence herein.

The inventive nucleic acids further comprise those nucleic acids coding for a polypeptide, whereby the polypeptide has or comprises a sequence, as designated herein with one or more of the “SEQ. ID. No.”, in particular SEQ. ID. No 2, 10. 17 and/or 5 to 8. Finally inventive nucleic acids are also those which comprise one or more exons and/or one or more of the introns of one or more of the herein disclosed inventive nucleic acids, in particular of the nucleic acids designated with den SEQ. ID. Nos. 1 and 3 and 4 and 9 and 11 to 16.

The inventive polypeptides, herein also designated in brief as polypeptides, are in particular translation products of one or more of the inventive nucleic acids or fragments thereof. At the same time the translation products can be present fully or abbreviated. These translation products are designated herein either generally as translation product, independent of the number, or as inventive translation product. Translation products or polypeptides in the sense of the present invention are in particular those parts of the corresponding polypeptides, which are part of a domain structure, in particular of such a domain as is present extracellular in a cellular system. This applies also for the fusion proteins, which can likewise be used as described hereinabove fully or abbreviated according to the present invention.

The inventive nucleic acids, in particular for the inventive DRIP, which in the prior art as such fragmentary already described are, can also be characterised or represented by the following nucleic acid sequences: The genomic sequence of 2p21-22, beginning on the BAC RP11 339H12- (AC010883), continuing via BAC RP11 183F15 (AC092838) or BACRP11 1069E24, ends with the last bp of BACRpll 204D19 (AC92615). The sequences are published with the access numbers (accession numbers of the GenBank databank) AC010883(RP11 339H12), AC092838(RP11 183F15), AC92615 (Rpll204D 19). The length of this genomic sequence is 506295 base pairs.

The herein disclosed sequences comprise human sequences in particular, showing:
SEQ. ID. No. 1 mRNA of the DRIP-FUS Ib splice variant mRNA,
SEQ. ID. No. 2 the DRIP-FUS Ib splice variant, the coding sequence (CDS) and therefore the amino acid sequence of the first fusion protein, whereby its DRIP portion comprises exons 1 to 28,
SEQ. ID. No. 3 the genomic sequence range, in which the FUS I component rests on chromosome 3,
SEQ. ID. No. 4 the genomic sequence range, in which the FUS II component rests on chromosome 7,
SEQ. ID. No. 5 the coding sequence (CDS) of DRIP and therefore the amino acid sequence, whereby DRIP comprises exons 1 to 38,
SEQ. ID. No. 6 the coding sequence (CDS) and therefore the amino acid sequence of the first fusion protein, FUS I, whereby its DRIP portion comprises exons 1 to 28,
SEQ. ID. No. 7 the coding sequence (CDS) and therefore the amino acid sequence of the splice variant of the first fusion protein (FUSIa), whereby its DRIP portion comprises exons 1 to 28,
SEQ. ID. No. 8 the coding sequence (CDS) and therefore the amino acid sequence of the second fusion protein (FUS II), whereby its DRIP portion comprises exons 1 to 28,
SEQ. ID. No. 9 the splice variant mRNA of the DRIP-FUS IIa fusion gene from thyroid gland tumor,
SEQ. ID. No. 10 the amino acid sequence of the splice variant of the DRIP-FUS IIa protein, for the SEQ. ID. Nos. 1 to 2 and 6 to 10 designated DRIP the part of DRIP, which comprises exons 1 to 28 and SEQ. ID. No. 5 that part which comprises exons 1 to 38.
   Furthermore,
SEQ. ID. No. 11 shows herein the DRIP-mRNA sequence comprising exons 1 to 38,
SEQ. ID. No. 12 the genomic sequence of DRIP comprising exons 1 to 38,
SEQ. ID. No. 13 the mRNA sequence of the DRIP-FUS I gene, whereby this comprises exons 1 to 28 of the DRIP gene as well as exons I, Ia and II of FUS I from the chromosome 3p25 range,
SEQ. ID. No. 14 the mRNA sequence of a splice variant of the DRIP-FUSI gene, whereby this comprises exons 1 to 28 of the DRIP gene as well as exon II of FUS I from the chromosome 3p25 range,
SEQ. ID. No. 15 FUSI gene mRNA sequence of the DRIP-FUSII gene, whereby this comprises exons 1 to 28 of the DRIP gene and exon A of FUS II from the chromosome 7p 15 range,
SEQ. ID. No. 16 FUSI gene mRNA sequence of the DRIP splice variant, whereby this comprises exons 1 to 26 and 29 to 38 of the DRIP gene,
SEQ. ID. No. 17 FUSI gene coding sequence (CDS) of FUSI gene DRIP splice variant and therefore FUSI gene amino acid sequence, whereby DRIP comprises exons 1 to 26 and 29 to 38.

It is within the scope of the present invention that the use, disclosed herein for one or more of the inventive polypeptides, proteins, nucleic acids or the compounds derived therefrom, as disclosed herein, in particular also a compound, such as small molecules, binding peptides, antibodies, anticalins and functional nucleic acids, contained within the scope of a screening process, which uses the latter individually, in combination or as fragment(s), is basically also disclosed in each case for the other abovementioned molecules or compounds.

The invention is explained hereinbelow by means of the figures, examples and the sequence protocols, from which further characteristics, embodiments and advantages of the invention will emerge, in which:

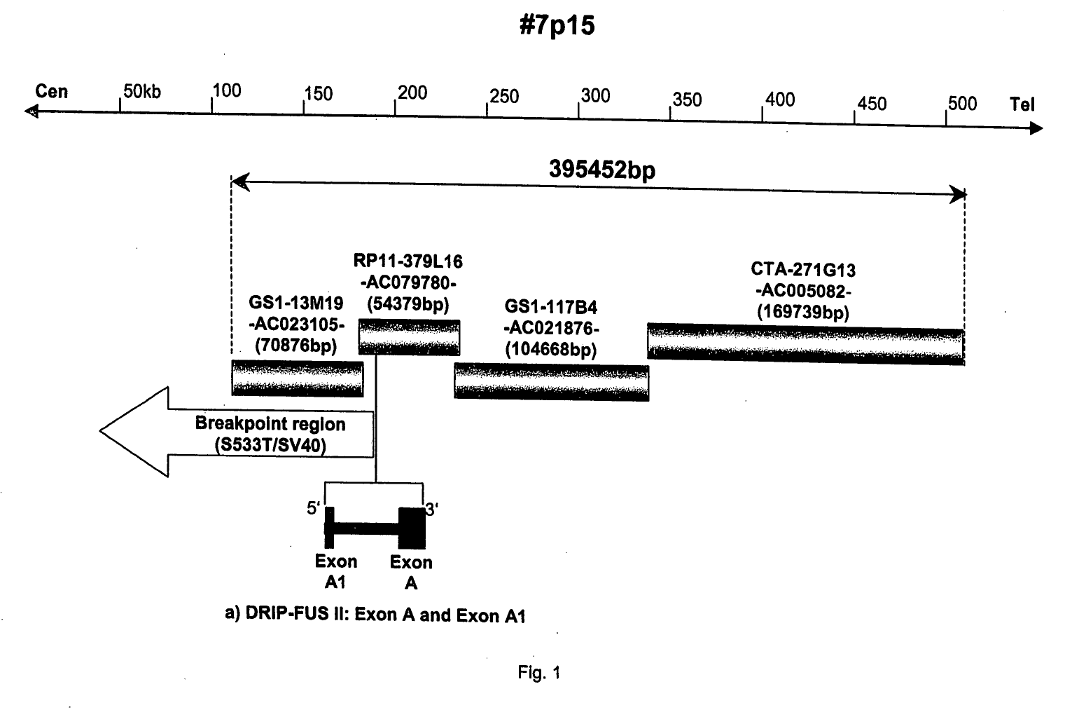

FIG. 1 is a schematic illustration of the genomic position of the breakpoint region on chromosome 7p 15;

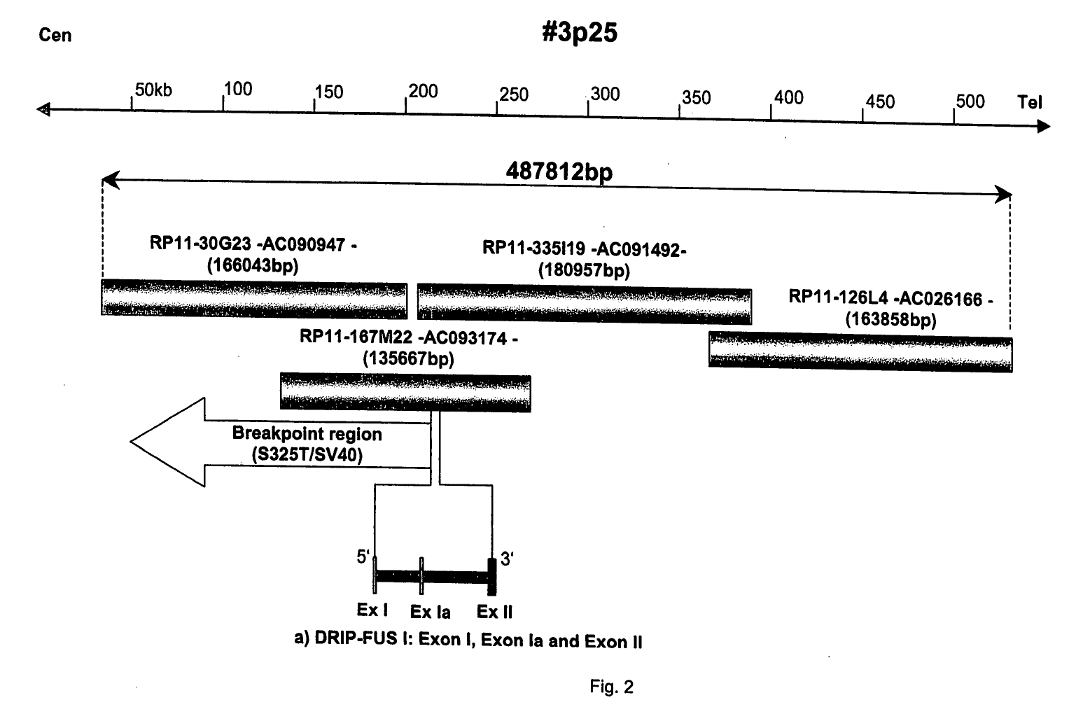

FIG. 2 is a schematic illustration of the genomic position of the breakpoint region on chromosome 3p25;

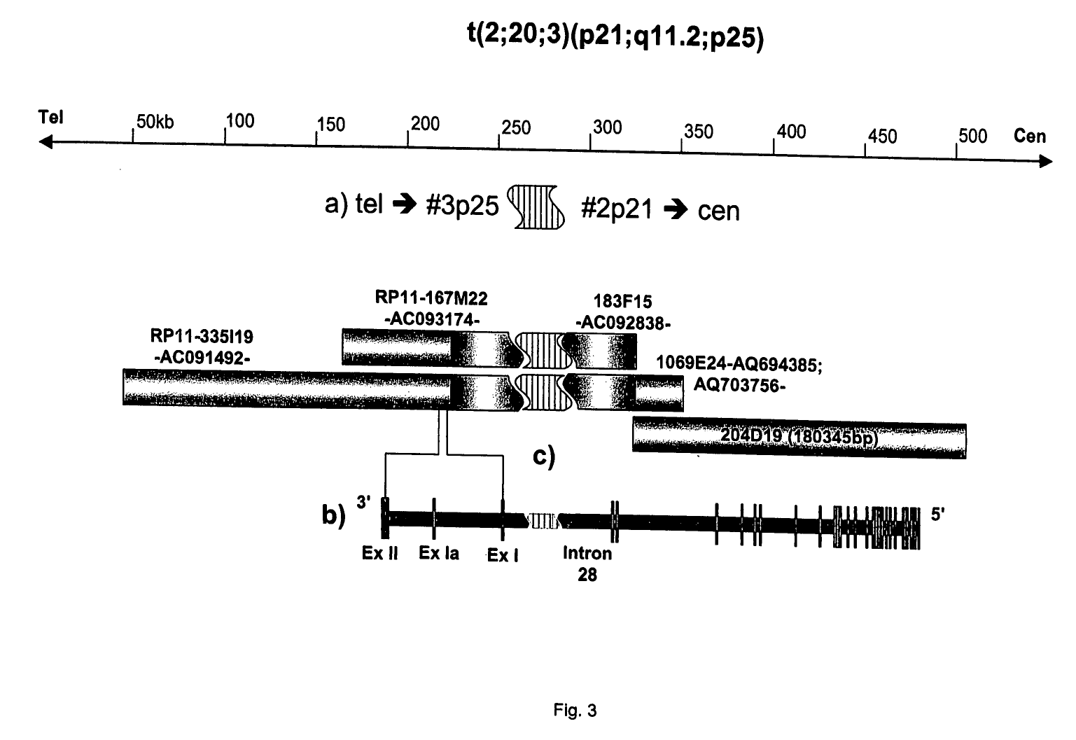

FIG. 3 is a schematic illustration of the genomic position of the breakpoint region on (2) in the cell line S325T/SV40 which has the fusion transcript DRIP-FUS I;

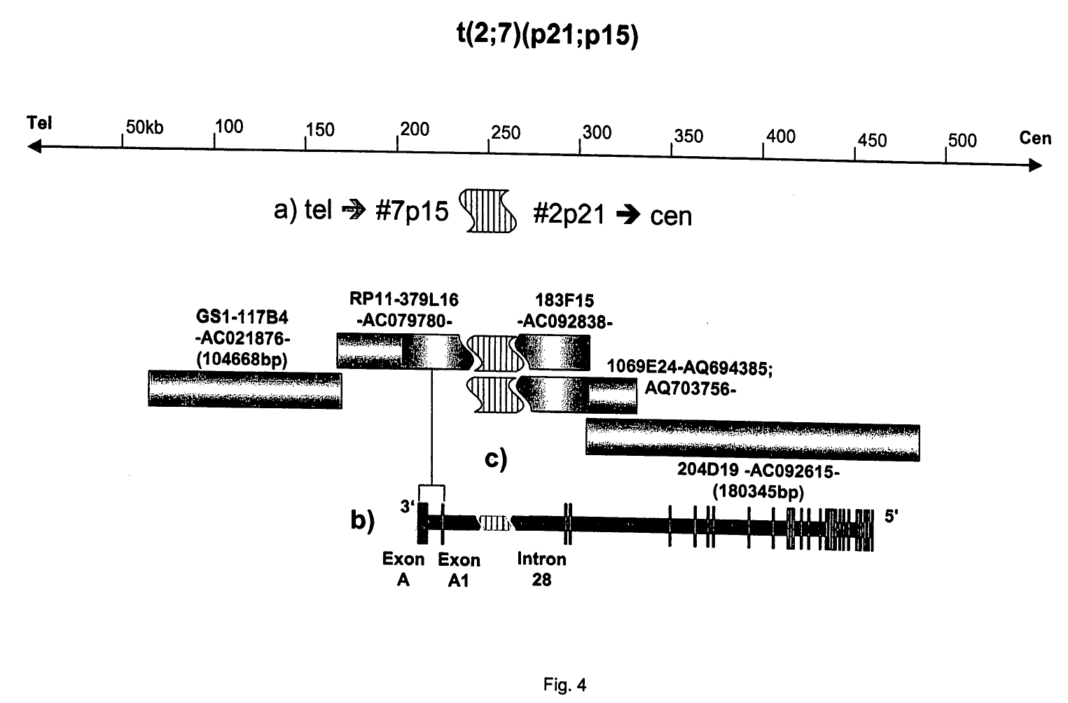

FIG. 4 is a schematic illustration of the genomic position of the breakpoint region on (2) in the cell line S533T/SV40 which has the fusion transcript DRIP-FUS II;

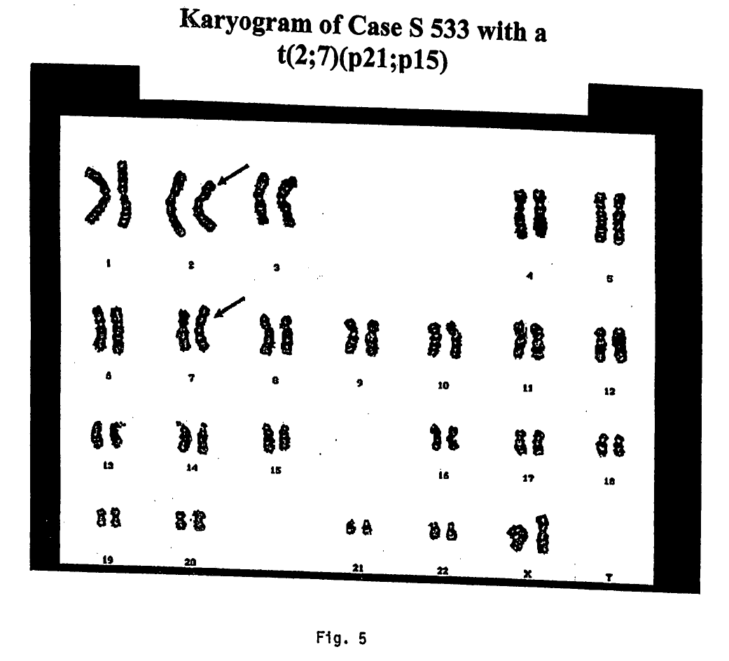

FIG. 5 is a karyogram of the primary tumor S 533, which has a translocation t (2; 7) (p21;pl5);

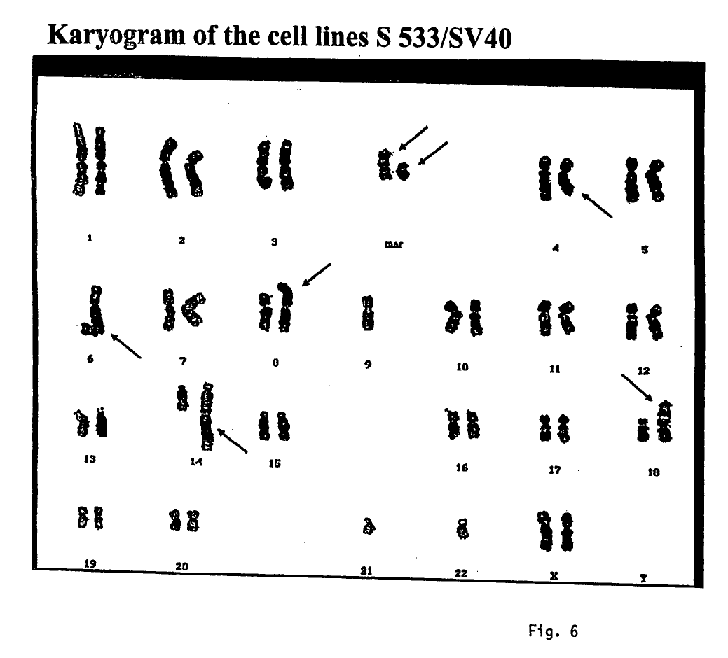

FIG. 6 is a karyogram of the cell line S 533 T/SV 40 produced from the primary culture S 533;

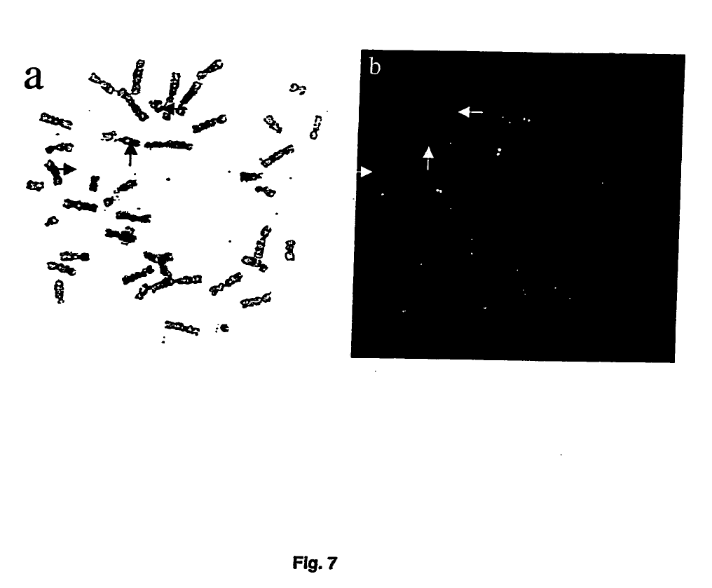

FIG. 7a is a metaphase of the cell line S325T/SV40 with a t (2; 3; 20) (p21; ql 1. 2; p25) after G banding;

FIG. 7b is a metaphase of the cell line S325T/SV40 with a t (2; 3; 20) (p21; qll. 2; p25) after FISH with BAC 1069E24, whereby the hybridising signals are localised on chromosome 2, the (2) and the (20), marked with arrows;

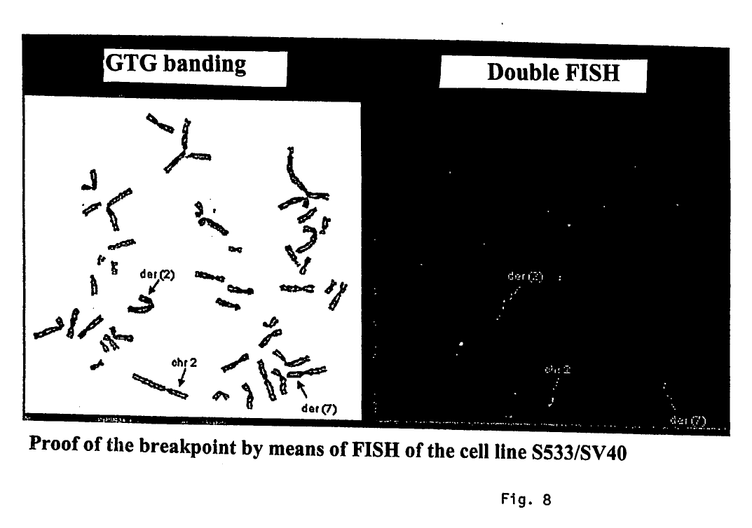

FIG. 8 is the result of the proof of the break point in the cell line S 533/TSV40 which leads to the fusion transcript DRIP-FUS II;

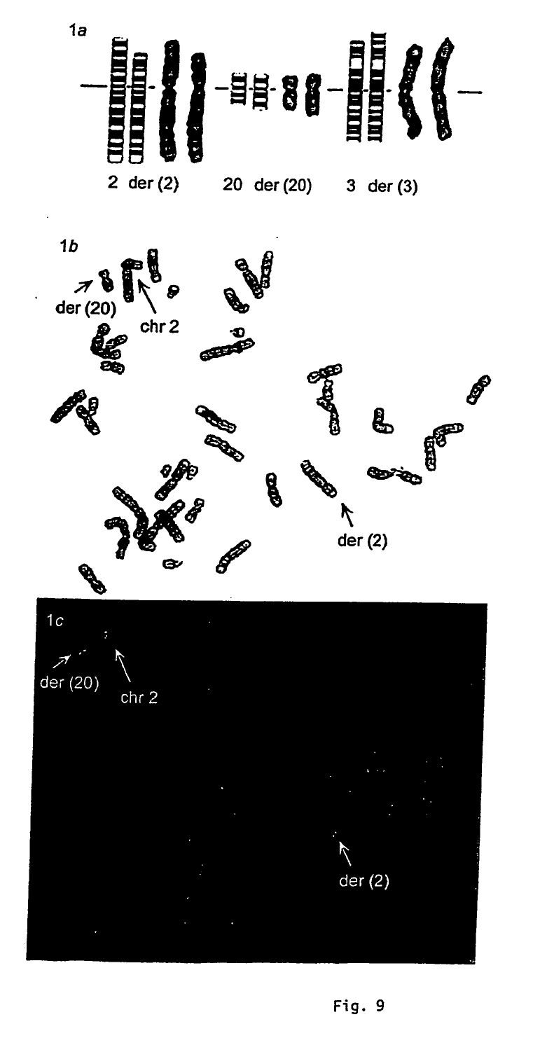

FIG. 9 is a karyogram and FISH results of the cell line S 325/TSV40;

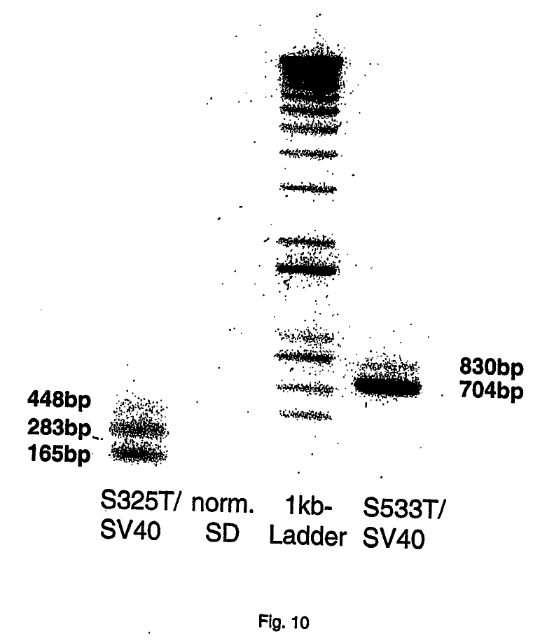

FIG. 10 is proof of the first and second fusion protein by means of RT-PCR;

FIG. 11 is the result of Northern-Blot analysis with use of a DRIP- specific probe;

FIG. 12 schematically illustrates the genomic position of DRIP relative to the break point cluster in different benign thyroid gland tumors and hyperplasias with 2p21 changes, whereby the translocation break at the cell lines S 325/TSV 40 and S 533/TSV 40 is localised in intron 28;

FIG. 13 schematically illustrates the DRIP-mRNA with its splice variant;

FIG. 14 is a schematic illustration of the three forms of the fusion transcript DRIP-FUSI; and

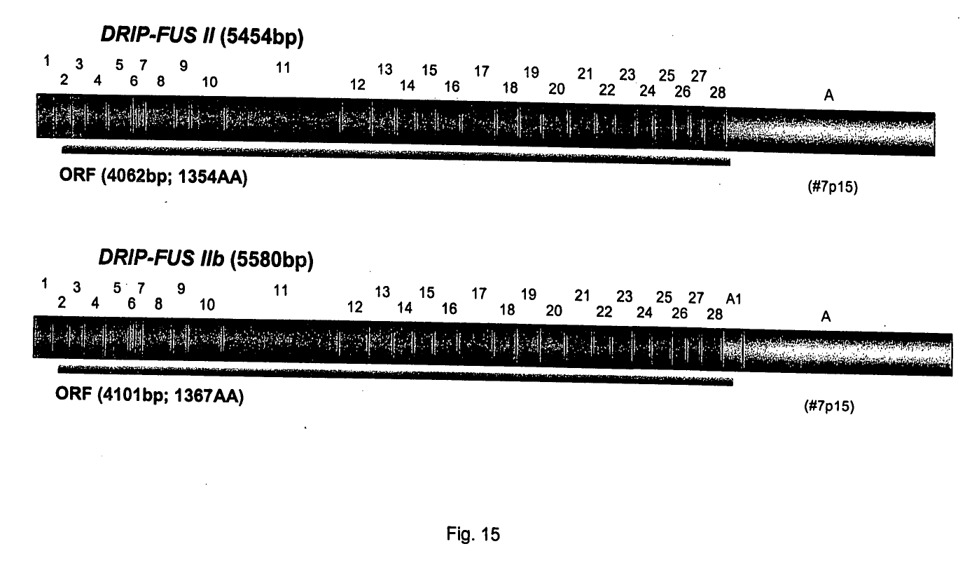

FIG. 15 is a schematic illustration of the two forms of the second fusion gene DRIP-FUS II.

In addition to the sequences specified in the sequence protocol the following is disclosed in reference thereto.

SEQ. ID. No. 1

SEQ. ID. No. 1 represents the fusion gene DRIP-FUS Ib (splice variant); proven in the cell line S325T/SV40 with a t (2; 20; 3)(p21; ql 1.2; p25).

The following positions of the mRNA sequence positions correspond to the described exons:

| Position on | | |
|---|---|---|
| the mRNA | | (no. of |
| seq | Exon no. | bp) |
| 1 . . . 108 | exon01 | 108 |
| 109 . . . 208 | exon02 | 100 |
| 209 . . . 303 | exon03 | 95 |
| 304 . . . 434 | exon04 | 131 |
| 435 . . . 583 | exon05 | 149 |
| 584 . . . 616 | exon06 | 33 |
| 617 . . . 665 | exon07 | 49 |
| 666 . . . 853 | exon08 | 188 |
| 554 . . . 948 | exon09 | 95 |
| 949 . . . 1169 | exon10 | 221 |
| 1170 . . . 1861 | exon11 | 692 |
| 1862 . . . 2040 | exon12 | 179 |
| 2041 . . . 2196 | exon13 | 156 |
| 2197 . . . 2319 | exon14 | 123 |
| 2320 . . . 2443 | exon15 | 124 |
| 2444 . . . 2595 | exon16 | 152 |
| 2596 . . . 2506 | exon17 | 211 |
| 2807 . . . 2942 | exon18 | 136 |
| 2943 . . . 3079 | exon19 | 137 |
| 3080 . . . 3238 | exon20 | 159 |
| 3239 . . . 3396 | exon21 | 158 |
| 3397 . . . 3506 | exon22 | 110 |
| 3507 . . . 3639 | exon23 | 133 |
| 3640 . . . 3753 | exon24 | 114 |
| 3754 . . . 3876 | exon25 | 123 |
| 3877 . . . 3968 | exon26 | 92 |
| 3969 . . . 4058 | exon27 | 90 |
| 4059 . . . 4190 | exon28 | 132 |
| 4191 . . . 4308 | DRIP-FUS I; | 118 |
| 4309 . . . 4473 | DRIP-FUS I; | 165 |
| 4474 . . . 4731 | DRIP-FUS I; | 258 |

SEQ. ID. No. 3

SEQ. ID. No. 3 represents the genomic sequence of chromosome 3p25, as it is contained apart from the DRIP sequence in the nucleic acid according to SEQ. ID. No. 13 and 14. The genomic sequence comprises in this case both exon I and also exon II of chromosome 3p25, whereby exon I takes up the nucleotide positions 81174 to 81291 and exon II the nucleotide positions 85616 to 85873 of SEQ. ID. No. 3. The genomic structure was taken from the BAC clone RP 11-167M22.

SEQ. ID. No. 4

SEQ. ID. No. 4 represents the genomic sequence range of FUS II exon A and A1 of chromosome 7p15, as it is part of the second fusion gene. The genomic sequence was obtained from the BAC clone RP 111-370L16. The exon A corresponds to the positions 7686 to 8949 of the genomic sequence according to SEQ. ID. No. 4.

SEQ. ID. No. 5

SEQ. ID. No. 5 represents the coding sequence (CDS) of DRIP and therefore the corresponding peptide, whereby DRIP comprises exons 1 to 38. It comprises a total of 1953 amino acids.

SEQ. ID. No. 6

SEQ. ID. No. 6 represents the coding sequence (cds) and therefore the amino acid sequence of the first fusion protein DRIP-FUS I, whereby its DRIP portion comprises exons 1 to 28. It comprises a total of 1387 amino acids.

SEQ. ID. No. 7

SEQ. ID. No. 7 represents the coding sequence (CDS) and therefore the amino acid sequence of the splice variant of the first fusion protein, whereby its DRIP portion comprises exons 1 to 28. It comprises a total of 1353 amino acids.

SEQ. ID. No. 8

SEQ. ID. No. 8 represents the coding sequence (CDS) and therefore the amino acid sequence of the second fusion protein, whereby its DRIP portion comprises exons 1 to 28. It comprises a total of 1353 amino acids.

SEQ. ID. No. 9

SEQ. ID. No. 9 represents the fusion gene DRIP-FUS IIa (splice variant); proven in the cell line S533T/SV40 with a t (2; 7) (p21;p15). The sequence length is 5580 bp.

The exons (here features) are localised as follows:

Features

| Array | Exon No. | No. of base pairs |
|---|---|---|
| exon 1 . . . 108 | Exon01 | 108 |
| exon 109 . . . 208 | Exon02 | 100 |
| exon 209 . . . 303 | Exon03 | 95 |
| exon 304 . . . 434 | Exon04 | 131 |
| exon 435 . . . 583 | Exon05 | 149 |
| exon 584 . . . 616 | Exon06 | 33 |
| exan 617 . . . 665 | Exon07 | 49 |
| exon 666 . . . 853 | Exon08 | 188 |
| exon 854 . . . 948 | Exon09 | 95 |
| exon 949 . . . 1169 | Exon10 | 221 |
| exon 1170 . . . 1861 | Exon11 | 692 |
| exon 1862 . . . 2040 | Exon12 | 179 |
| exon 2441 . . . 2196 | Exon13 | 156 |
| exon 2197 . . . 2319 | Exon14 | 123 |
| exon 2320 . . . 2443 | Exon15 | 124 |
| exon 2444 . . . 2595 | Exon16 | 152 |
| exon 2596 . . . 2806 | Exon17 | 211 |
| exon 2807 . . . 2942 | Exon18 | 136 |
| exon 2943 . . . 3079 | Exon19 | 137 |
| exon 3080 . . . 3238 | Exon20 | 159 |
| exon 3239 . . . 3396 | Exon21 | 158 |
| exon 3397 . . . 3506 | Exon22 | 110 |
| exon 3507 . . . 3639 | Exon23 | 133 |
| exon 3640 . . . 3753 | Exon24 | 114 |
| exon 3754 . . . 3876 | Exon25 | 123 |
| exon 3877 . . . 3968 | Exon26 | 92 |
| exon 3969 . . . 4058 | Exon27 | 90 |
| exon 4059 . . . 4190 | Exon28 | 132 |
| exon 4191 . . . 4316 | Chr. 7p15 | DRIP-FUS II Exon A1 |
| exon 4317 . . . 5580 | Chr. 7p15 | DRIP-FUS II Exon A |

SEQ. ID. No. 10 represents the coding sequence (CDS) and therefore the amino acid sequence of the splice variant of the second fusion protein DRIP-FUSIIa, whereby its DRIP portion comprises exons 1 to 28. It comprises a total of 1366 amino acids.

SEQ. ID. No. 11

SEQ. ID. No. 11 represents the mRNA of DRIP comprising exons 1 to 38. The length of mRNA is 6090 bp. The open in-frame (ORF) begins with position 133 and ends with position 5994. The following primers, which hybridise at different places of the mRNA, can be used as proof of mRNA.

| | |
|---|---|
| Primer Name | Sequence |

| | |
|---|---|
| DRIPVRU1 | 5′ATGGGAGAACCAAATCGTCATCCAAGCATG 3′ |
| DRIPVRL2 | 5′TCAACATGCCGCTTCTGTTCTTGGAAGAGTTAA 3′ |
| DRIPVRU3 | 5′TTTCCGGCAAAACCACATTCATGGGACACT 3′ |
| DRIPVRL4 | 5′TACTGGGGCAGGCCTGGCACCTTGAAG 3′ |
| DRIPVRU5 | 5′TGGCCGTCGTTGAAGTCCTCACCAGT 3′ |
| DRIPVRL6 | 5′GCCAGCCGGACTTTGAGAGGAGACAGAAG 3′ |
| DRIPVRU9 | 5′GCTTCAGGCAGCAGCAGCATTTCCA 3′ |
| DRIPVRL10 | 5′ATTGGGATGAGGCCTTCAGGGGATGA 3′ |
| DRIPVRL10 | 5′ATTGGGATGAGGCCTTCAGGGGATGA 3′ |
| DRIPVU11 | 5′CCTGCCCCAGTACCTCCAGAGCCTCAC 3′ |
| DRIPVL12 | 5′GAACTCCTTTGGGGTCAGATGGACACAGTG 3′ |
| DRIPVU13 | 5′ACTGCATGGACCCTGGTGAGTGGCT 3′ |
| DRIPVL14 | 5′GCATGTGGTGGGAAATGACTTTGGAAG 3′ |
| DRIPVU15 | 5′CTCAGCACAAGCACCAAACCATACGACTGT 3′ |
| DRIPVU16 | 5′AGAGTAAATCCAAACGTGAACCAGAGAATGAGT 3′ |
| DRIPVRU17 | 5′GGCTGGGAGAAAATATTATTCCTTATGTTG 3′ |
| DRIPVU18 | 5′GTCTGGGCAGTGCGAAATTCATCCAC 3′ |
| DRIPVU19 | 5′AGACTCTACGCTTCCCCGATGGATGGT 3′ |
| DRIPL20 | 5′CAGGCGCGTATCTCTGAACAATGCTCTAAG 3′ |
| DRIPVL21 | 5′GAAGGGTGCTGTTTGGTTAACTCATTCTC 3′ |
| DRIPVU22 | 5′GCGAATAGCTAGAGCTCATGGACATCT 3′ |
| DRIPVU23 | 5′ATTTTTACCAAGGTTTTAAGCGATGATGA 3′ |
| DRIPVL24 | 5′AGTGAGCCGGTGCATTTGGACAAG 3′ |
| DRIPVU25 | 5′CTCAGACGCCGACGTGCACGAGTGACTA 3′ |
| DRIPVL26 | 5′ACGAGAAGTAAAACGGTGCATAGCCTCAGGT 3′ |
| DRIPVU27 | 5′GGATATCTTAGCAGGCATTTATCTTTCTTTGAGTCT 3′ |
| DRIPVL28 | 5′ATATTTTGCCATATGGGAGAATCGGAAGTC 3′ |
| FOSTSWI14187 | 5′ATTTATGTTCAACATGTTTCCTGC 3′ |
| RVSTSWI14187 | 5′TGCAGAATCGAGGCAGTTAA 3′ |

SEQ. ID. No. 12

The SEQ. ID. No. 12 represents the genomic sequence of the DRIP gene on chromosome 2p21-22.

The length of the genomic DNA of DRIP, which comprises exons 1 to 38, is 365186 bp.

The following exon positions can be specified in genomic DNA:

| Position in genomic | Exon No. | No. of exon |
|---|---|---|
| 1 . . . 108 | Exon01 | 08 |
| 3623.3722 | Exon02 | 100 |
| 3993 . . . 4087 | Exon03 | 95 |
| 5085 . . . 5215 | Exon04 | 131 |
| 9037 . . . 9185 | Exon05 | 149 |
| 9305 . . . 9337 | Exon06 | 33 |
| 9590 . . . 9638 | Exon07 | 49 |
| 14110 . . . 14297 | Exon08 | 188 |
| 17431 . . . 17525 | Exon09 | 95 |
| 18796 . . . 19016 | Exon10 | 221 |
| 21011 . . . 21702 | Exon11 | 692 |
| 23046 . . . 23224 | Exon12 | 179 |
| 24176 . . . 24331 | Exon13 | 156 |
| 25528 . . . 25650 | Exon14 | 123 |
| 29217 . . . 29340 | Exon15 | 124 |
| 35653 . . . 35804 | Exon16 | 152 |
| 39483 . . . 39693 | Exon17 | 211 |
| 43699 . . . 43834 | Exon18 | 136 |
| 44113 . . . 44249 | Exon19 | 137 |
| 46670 . . . 46828 | Exon20 | 159 |
| 54722 . . . 54879 | Exon21 | 158 |
| 66890 . . . 68159 | Exon22 | 1270 |
| 68050 . . . 68159 | Exon22 | 110 |
| 87258 . . . 87390 | Exon23 | 133 |
| 90303 . . . 90416 | Exon24 | 114 |
| 97083 . . . 97205 | Exon25 | 123 |
| 110713 . . . 110804 | Exon26 | 92 |
| 165736 . . . 165825 | Exon27 | 90 |
| 167807 . . . 167938 | Exon28 | 132 |
| 197899 . . . 198067 | Exon29 | 169 |
| 251801 . . . 251916 | Exon30 | 116 |
| 275498 . . . 275592 | Exon31 | 95 |
| 302825.303204 | Exon32 | 380 |
| 303816 . . . 303934 | Exon33 | 119 |
| 304270 . . . 304342 | Exon34 | 73 |
| 308977 . . . 309130 | Exon35 | 154 |
| 316142 . . . 316273 | Exon36 | 132 |
| 363156 . . . 363325 | Exon37 | 170 |
| 364695 . . . 365186 | Exon38 | 492 |

The following primers, which hybridise at different places of the mRNA, can be used as proof of mRNA.

| | |
|---|---|
| Primer Name | Sequence |

| | |
|---|---|
| DRIPVRU1 | 5′ATGGGAGAACCAAATCGTCATCCAAGCATG 3′ |
| DRIPVRL2 | 5′TCAACATGCCGCTTCTGTTCTTGGAAGAGTTAA 3′ |
| DRIPVRU3 | 5′TTTCCGGCAAAAGCACATTCATGGGACACT 3′ |
| DRIPVRL4 | 5′TACTGGGGCAGGCCTGGCACCTTGAAG 3′ |
| DRIPVRU5 | 5′TGGCCGTCGTTGAAGTCCTCACCAGT 3′ |
| DRIPVRL6 | 5′GCCAGCCGGACTTTGAGAGGAGACAGAAG 3′ |
| DRIPVRU7 | 5′CTGGCAAATTCCATGGTTCTGTCTTGAAGTGA 3′ |
| DRIPVRL8 | 5′CAATGGAACCAGAACATGAAGCCAAGCAGTCT 3′ |
| DRIPVRU9 | 5′GCTTCAGGCAGCAGCAGCATTTCCA 3′ |
| DRIPVRL10 | 5′ATTGGGATGAGGCCTTCAGGGGATGA 3′ |
| DRIPVU11 | 5′CCTGCCCCAGTACCTCCAGAGCCTCAC 3′ |
| DRIPVL12 | 5′GAACTCCTTTGGGGTCAGATGGACACAGTG 3′ |
| DRIPVU13 | 5′ACTGCATGGACCCTGGTGAGTGGCT 3′ |
| DRIPVL14 | 5′GCATGTGGTGGGAAATGACTTTGGAAG 3′ |
| DRIPVU15 | 5′CTCAGCACAAGCACCAAACCATACGACTGT 3′ |
| DRIPVU16 | 5′AGAGTAAATCCAAACGTGAACCAGAGAATGAGT 3′ |
| DRIPVRU17 | 5′GCCTGGGAGAAAATATTATTCCTTATGTTG 3′ |
| DRIPVU18 | 5′GTCTGGGCAGTGCGAAATTCATCCAC 3′ |
| DRIPVU19 | 5′AGACTCTACGCTTCCCCGATGGATGGT 3′ |
| DRIPL20 | 5′CAGGCGCGTATCTCTGAACAATGCTCTAAG 3′ |
| DRIPVL21 | 5′GAAGGGTGCTGTTTGGTTAACTCATTCTC 3′ |
| DRIPVU22 | 5′GCGAATAGCTAGAGCTCATGGACATCT 3′ |
| DRIPVU23 | 5′ATTTTTACCAAGGTTTTAAGCGATGATGA 3′ |
| DRIPVL24 | 5′AGTGAGCCGGTGCATTTGGAGAAG 3′ |
| DRIPVU25 | 5′CTCAGACGCCGACGTGCACGAGTGACTA 3′ |
| DRIPVL26 | 5′ACGAGAAGTAAAACGGTGCATAGCCTCAGGT 3′ |
| DRIPVU27 | 5′GGATATCTTAGCAGGCATTTATCTTTCTTTGAGTCT 3′ |
| DRIPVL28 | 5′ATATTTTGCCATATGGGAGAATCGGAAGTC 3′ |
| DRIPVU29 | 5′TAGCATCCTGGACTAATTCAGCCATAGAAG 3′ |
| DRIPVU30 | 5′GACGGTGGAGCAGGTAAAAGAAATAGG 3′ |
| DRIPVL31 | 5′GCAGCTGGGGAGTGTGGACAAC 3′ |
| DRIPVU32 | 5′TAATTCATCACCATTGCCAAGTAAGGATAG 3′ |
| DRIPVL33 | 5′GGACATCAAA.AGGAGCTTCTCTACCAC 3′ |
| DRIPVU34 | 5′AGTGGAGAGCGGGAGACGAATG 3′ |
| DRIPVL35 | 5′AAGAATAGGATGGGGGTTGGTGAG 3′ |
| DRIPVU36 | 5′AGCCTAACGGACAGCCTGAATGGGA 3′ |
| FOSTSWI14187 | 5′ATTTATGTTCAACATGTTTCCTGC 3′ |
| RVSTSWT14187 | 5′TGCAGAATCGAGGCAGTTAA 3′ |

The sequences of exons 1 to 38 can be taken from SEQ. ID. No. 11.

SEQ. ID. No. 13

SEQ. ID. No. 13. comprises the mRNA sequence of the DRIP-FUS I gene, whereby the latter comprises exons 1 to 28 of the DRIP gene and exons I, Ia and II of chromosome 3p25.

The total length of mRNA is 4558 bases. The arrangement of exons 1 to 28 of that part of mRNA, originating from DRIP, corresponds to the arrangement of exons 1 to 28 in DRIP, as is also shown in SEQ. ID. No. 11 and 12. Exon I of chromosome 3p25 comprises nucleotides 4191 . . . 4308 and exon II of chromosome 3p25 comprises nucleotides 4309 . . . 4566 of the sequence according to SEQ. ID. No. 13. The fusion gene DRIP-FUS I was proven in the cell line S325T/SV 40 with a t(2; 20; 3) (p21;qll. 2; p25). The following primers were used as proof of the nucleic acid according to SEQ. ID. No. 13:

| | |
|---|---|
| Primer | Sequence |

| | |
|---|---|
| DRIPVRU1 | 5′ATGGGAGAACCAAATCGTCATCCAAGCATG 3′ |
| DRIPVRU9 | 5′GCTTCAGGCAGCAGCAGCATTTCCA 3′ |
| DRIPVRL10 | 5′ATTGGGATGAGGCCTTCAGGGGATGA 3′ |
| DRIPVU15 | 5′CTCAGCACAAGCACCAAACCATACGACTGT 3′ |
| DRIPVU16 | 5′AGAGTAAATCCAAACGTCAACCAGAGAATGACT 3′ |
| DRIPVRU17 | 5′GCCTGGGAGAAAATATTATTCCTTATGTTG 3′ |
| DRIPVU18 | 5′GTCTGGGCAGTGCGAAATTCATCCAC 3′ |
| DRIPVU19 | 5′AGACTCTACGCTTCCCCGATGGATGGT 3′ |
| DRIPL20 | 5′CAGGCGCGTATCTCTGAACAATGCTCTAAG 3′ |
| DRIPVL22 | 5′GAAGGGTGCTGTTTGGTTAACTCATTCTC 3′ |
| DRIPVU22 | 5′GCGAATAGCTAGAGCTCATGGACATCT 3′ |
| DRIPVU23 | 5′ATTTTTACCAAGGTTTTAAGCGATGATGA 3′ |
| DRIPVL24 | 5′AGTGAGCCGGTGCATTTGGAGAAG 3′ |
| DRIPVU25 | 5′CTCAGACGCCGACGTGCACGAGTGACTA 3′ |
| DRIPVL26 | 5′ACGAGAAGTAAAACGGTGCATAGCCTCAGGT 3′ |
| DRIPVU27 | 5′GGATATCTTAGCAGGCATTTATCTTTCTTTGAGTCT 3′ |
| DRIPVL28 | 5′ATATTTTGCCATATGGGAGAATCGGAAGTC 3′ |
| FUSVL01 | 5′TGCTTTGGGAGCCAGGTCACTGAGTTACTAC |
| FUSVL02 | 5′ACTGCTTTGGGAGCCAGGTCACTGAGT |
| FUSVL03 | 5′TCCAGGGAAATTCACTGCTTTGGGAGCCA |

SEQ. ID. No. 14

SEQ. ID. No. 14 shows the mRNA sequence of a splice variant of the DRIP-FUSI gene, proven in the cell line S325T/SV40 with a t (2; 20; 3) (p21; qll. 2; p25), whereby this comprises exons 1 to 28 of the DRIP gene and exon II, with position 4191 to 4448 of the MRNA sequence, of chromosome 3p25. This splice variant was obtained from the same cell line as described in connection with SEQ. ID. No. 13. The mRNA in this case has a length of 4448 bases. The genomic sequence of the part of the nucleic acids according to SEQ. ID. No. 13 and 14, which are contributed by DRIP to the respective fusion gene, correspond to the ranges, as disclosed in connection with SEQ. ID. No. 12.

SEQ. ID. No. 15

SEQ. ID. No. 15 shows the mRNA sequence of the second fusion gene, which is herein also designated as DRIP-FUS II, which for one comprises exons 1 to 28 of the DRIP gene and secondly comprises exon A of #7pl5. The length of mRNA is 5454 bases, whereby the first 4190 bases correspond to those of mRNA of DRIP and bases 4191 to 5454 correspond to exon A of #;7pl5. The corresponding primers are suitable for proof of exons 1 to 28 of the DRIP gene, as were described in connection with SEQ. ID. No. 13. The following primers can be used for proof of the sequence portion, which is attributed to #;7p15 at the following nucleotide positions of SEQ. ID. No. 16:

| | |
|---|---|
| Primer Name | Sequence |

| | |
|---|---|
| FUSIIL01 | 5′GGTAGCGGGAGCAATCACAAAACTGTAA |
| FUSIIL02 | 5′CTCTTCTTTTGATAGGACAGCCCTTGTTCTGA |
| FUSIIL03 | 5′GACCGCTTCTTGCAGAGGCTGAGAGTC |

SEQ. ID. No. 16

SEQ. ID. No. 16

SEQ. ID. No. 16 shows the mRNA sequence of the DRIP splice variant. The open in-frame (ORF) begins with position 133 and ends with position 5772.

Complementary to the explanations made in the overview of the figures the following is also revealed with reference to the individual figures.

FIG. 1 is a schematic illustration of the genomic position of the breakpoint region on chromosome 7, more precisely #;7pl5. The break point is significant for providing that part of the fusion transcript DRIP-FUS II, which stems from chromosome 7. The result was determined on the cell line S533/TSV40. The length of the breakpoint region is not defined in terms of the telomer. Exon A, as in DRIP-FUS II, can be obtained from the BAC clone RP 11-379L16 (AC 079780). Exon A and A1 is illustrated on an enlarged scale relative to the BAC clone in FIG. 1a).

FIG. 2 is a schematic illustration of the genomic position of the breakpoint region on chromosome 3, more precisely #;3p25. The break point is significant for providing that part of the fusion transcript DRIP-FUS I, which stems from chromosome 3. The results were determined on the cell line S325T/SV40. The length of the breakpoint region is not defined in terms of telomer. The exons 1 Ia and II of chromosome 3, which are contained in the fusion transcript DRIP-FUS I, can be obtained from the BAC clones RP 11-167M22 (AC093174) and RP11-33519 (AC091492). The exons I Ia and II are illustrated in FIG. 2a) on en enlarged scale relative to the BAC clone.

FIG. 3 is a schematic illustration of the genomic position of the breakpoint region on (2) with cell line S325T/SV40. which has the fusion transcript DRIP-FUS I. At the same time FIG. 3a) shows the description of the chromosomal sections on (2) and FIG. 3b) shows the genomic structure of DRIP-FUS I, whereby exon I (Ex I), exon Ia and exon II (Ex II) are arranged at the 3′ end and are represented on an enlarged scale. As illustrated in FIG. 3c), the size of the break point range between exon I (#;3p25) and exon 28 (#;2p21) is not defined and therefore a length detail to adjoining BAC clones is not possible. The BAC clones are taken from the BAC library RP 11 and can be identified by their identification number and the gene bank access number.

FIG. 4 is a schematic illustration of the genomic position of the breakpoint region on (2) with cell line S533T/SV40. which has the fusion transcript DRIP-FUS II. At the same time FIG. 2a) shows the description of the chromosomal sections on (2) and FIG. 2b) shows the genomic structure of DRIP-FUS II, whereby exon A (Ex A) and exon AI(ExAl) are arranged at the 3′ end and are represented on an enlarged scale. As illustrated in FIG. 4c), the size of the break point range between exon A(#;7p15) or exon AI and exon 28 #;2p21) is not defined and therefore a length detail to adjoining BAC clones is not possible. The BAC clones are taken from the BAC library RP 11 and can be identified by their identification number and the gene bank access number.

FIG. 5 shows the karyogram of the primary tumor S 533 produced according to standard methods, which has a translocation t (2; 7) (p21;pl5). The translocations are characterised by arrows.

FIG. 6 shows the karyogram of the cell line S 533/TSV 40 produced from the primary culture S 533 according to standard methods. At the same time it is evident that the translocations evident in the primary tumor also remain intact in the cell line established from the primary tumor.

FIG. 8 shows the result of the proof of the break point in the cell line S 533/TSV40. which leads to the fusion transcript DRIP-FUS II. At the same time a so-called double FISH is carried out on the cell line S 533/TSV40 with the BAC clones RP 11-339H12 and RP 11-204 D19. The result is illustrated in the right partial figure. At the same time GTG dyeing of the band n-pattern of the chromosomes was carried out. The results of the double FISH are in accord with the result of the GTG dyeing, as shown by the arrows in both diagrams pointing to the break points.

FIG. 9 shows a karyogram and FISH results of the cell line S 325/TSV40. The karyogram was produced according to standard methods familiar to specialists in the field or the FISH according to standard processes. FIG. 9a) illustrates a part of the Giemsa-dyed karyotype and the suitable ideogram of the thyroid gland adenoma cell line S 325/TSV40. The cell line exhibits a translocation t (2; 20; 3) (p21;q11. 2; p25). In each case the normal state is illustrated to the left and the derived chromosomes 2, 20 and 3 are illustrated to the right. FIG. 9b) shows a metaphase of the cell line S 325/TSV40 with translocation t (2; 20; 3) (p21;q11. 2; p25) after Giemsa dyeing; chromosomes 2, the (2) and the (20) are indicated by arrows; FIG. 9c) shows the same metaphases according to FISH with BAC 1069E24, whereby the hybridising signals are arranged on chromosome 2, the (2) and the (20) are marked by arrows.

FIG. 10 shows the result of a RT-PCR, whereby the fusion transcript of the first fusion protein DRIP-FUS I was obtained from the cell line S 325 and the fusion transcript of the second fusion protein DRIP-FUS II was obtained from the cell line S533. The arrows show the position of the respective transcripts, whereby in the case of DRIP-FUS 1 the three splice variants were obtained and with DRIP-FUS II two splice variants were obtained. The trace marked with M is the reference trace, which contains different length standards.

FIG. 11 shows the result of Northern-Blot analysis using a DRIP-specific probe. For this RNA, which had been removed from the different specified tissues, was applied to a surface and hybridised with a DRIP-specific probe. The probe had a length of 880 pb and originated from exons 25 to 32 of the DRIP gene. The probe used corresponded to the primer DRIPVRU17/VRL4.

FIG. 12 shows the genomic position of DRIP relative to the break point cluster with different benign thyroid gland tumors and hyperplasias with 2p21-22 changes. The genomic sequence of DRIP can be represented by the overlapping BAC clones 339H12 (AC 010883), 183F15 (AC 092838), 1069E24 (AQ694385; AQ 703756) and 204D19 (AC 092615). The translocation break of the cell lines S 325/TSV 40 and S533/TSV 40 (herein also designated as S 533) occurs in intron 28. A break in this area results in both fusion proteins DRIP-FUS I and DRIP-FUS II.

FIG. 13 is a schematic illustration of the DRIP-mRNA whereby the DRIP-mRNA comprising 6090 bp comprises all 38 exons in all and the open in-frame (ORF) resulting therefrom has a length of 5862 bp. The ORF begins in exon 2 and extends in 3′-direction to exon 38 of the DRIP gene.

Also, a splice variant of the DRIP gene is illustrated in FIG. 13, in which exons 27 and 28 are deleted. This variant of the DRIP gene also represents an inventive nucleic acid or the peptide coded thereby represents an inventive peptide. The ORF of the splice variant comprises a total of 5640 bp and begins likewise in exon 2 and extends as far as exon 38.

FIG. 14 is a schematic illustration of three forms of the fusion transcript DRIP-FUS I. The first form comprises exons 1 to 28 of the DRIP gene and exons I and II of band 25 of chromosome 3 (herein also designated as #;3p25, or also chromosome 3p25). The length of this form is 4566 bp.

The ORF begins in exon 2 and extends as far as exon I of #;3 p25. The ORF itself is formed by 4164 bp in all according to 1388 amino acid radicals.

The second form of the first fusion transcript DRIP-FUS II comprises likewise exons 1 to 28 of the DRIP gene, however only exon II of #;3p25. This form has a length of 4448 bp, whereby the ORF again begins in exon 2 and extends as far as exon II. The ORF has a length of 4062 bp according to 1354 amino acid radicals.

The third form comprises exons 1 to 28 of the DRIP gene and exons I, Ia and II of band 25 of chromosome 3 (herein also designated as 3p25, or chromosome 3p25).

The length of this form is 4731 bp. The ORF begins in exon 2 and extends to exon I of #;3 p25. The ORF itself is formed by 4164 bp in all according to 1388 amino acid radicals.

FIG. 15 is a schematic illustration of the second fusion gene DRIP-FUS II, found in the cell line S 533/TSV40. The first fusion gene transcript DRIP-FUS II comprises 5454 bp in all and is formed by exons 1 to 28 of the DRIP gene and exon A of the band p15 of chromosome 7, which is arranged at the 3′ end of the fusion transcript. The ORF begins in exon 2 and extends partially into exon A. The ORF comprises a total of 4062 bp according to 1354 amino acid radicals.

The splice variant DRIP-FUS IIa comprises 5580 bp in all and is formed by exons 1 to 28 of the DRIP gene and exon AI and A of the band p15 of chromosome 7, which is arranged at the 3′ end of the fusion transcript. The ORF begins in exon 2 and extends partially into exon A1. The ORF comprises a total of 4101 bp according to 1367 amino acid radicals.

In so far as nothing contrary is specified herein, the specified sequences are human sequences, preferably isolated sequences and preferably isolated human sequences.

The herein disclosed sequences were reproduced in the sequence protocol and represent a substantial part of the description.

The invention is explained hereinbelow in greater detail with reference to the examples, from which further characteristics, embodiments and advantages of the present invention will emerge both from the figures and the sequence protocol.

### EXAMPLE 1

Moleculargenetic Examination of Thyroid Gland Adenomas with 2p21-

Aberrations

Structural aberrations with participation of the chromosome area 2p21 characterise a cytogenetic subgroup in the case of benign thyroid gland tumors. For identifying this break point two cell lines were established from benign thyroid gland tumors with 2p21 translocations. 18 BAC clones were added to these cell lines and an additional primary tumor for FISH studies. All break points were localised inside an area of approximately 450 kb.

The coincidence of the breakpoint region in the established cell lines and the primary tumor confirms the portability of the data on primary tumors obtained from the cell lines.

Material and Methods

After the operation a piece of tumor tissue was placed unfixed in sterile Hank's solution with 2% antibiotics (200 IU/ml Penicillin, 200 μg/ml Streptomycin). The tumor tissue was mechanically comminuted and treated for tissue exposure for 3-4 h with 0.35% collagenase (Serva, Heidelberg, Germany) for cultivating the cells. The cell suspension in an enzyme solution was centrifuged and the cell pellet was cultivated in culture medium with 20% foetal calf serum and 2% antibiotics (TC 199 with Earle's salts with 20% foetal calf serum and 2% antibiotics, 200 IU/ml Penicillin, 200 gg 7 ml Streptomycin). The chromosome preparation took place according to one of the methods described for pleomorphic adenoma of the thyroid gland (Bullerdiek et al., 1987). The karyotype description was done after the ISCN (1995).

The cell lines were established by means of transfection with an SV40 plasmide (SV40 “early region”) (Kazmierczak et al., 1990; Belge et al., 1992). Two cell lines and a primary tumor were used for FISH studies. The karyotype of one of the primary tumors has already been published (Bol et al., 1999). All tumors used in this study are characterised by clonal aberration of 2p21.

18 BAC clones were used for cloning the break point, which were isolated from the RPCI-11 library at the Resource Centre for Genome Research (Berlin, Germany). They were isolated by means of QIAGEN Plasmid Midi kit (QIAGEN, Hilden, Germany).

The fluorescence in situ hybridising (FISH) took place in metaphases, which were banded previously for identifying the GTG chromosomes. The metaphases and the FISH were isolated according to a protocol modified by Wanschura et al. (1995) by Kievits et al. (1990).

BAC-DNA was marked with Biotin-14-dUTP and digoxigenein-14-dUTP by means of Nick translation (Gibco BRL, Life Technologies, Eggenstein, Germany). For each BAC in each case approximately 10 metaphases of the cell lines and primary tumors were analysed. Analysis of the chromosomes was performed following counter-dyeing with DAPI or propidium iodide on the Zeiss Axiophot fluorescence microscope with a FITC filter (Zeiss, Oberkochem, Germany).

The results were recorded with the Power Gene karyotyping system (PSI, Halladale, U.K.).

Results

The fluorescence in situ hybridising studies were first carried out on both the established cell lines. 18 BAC clones, which are localised in the chromosome band 2p21, were used for FISH analyses. 10 GTG-banded metaphases were analysed for each BAC. After the break point region in both cell lines was narrowed between the BAC clones 339H12 and 1069E24, the primary tumor of one of the cell lines and a further primary tumor were tested. FISH analyses demonstrate that the break points of the examined cell lines and the primary tumors are localised between the BAC clones 339H12 and 1069E24.

Discussion

In the literature to date reports have been made on 450 cytogenetic examinations of thyroid gland hyperplasias and adenomas. Approximately 20% of these lesions show chromosomal changes, divided into various subgroups. 19q 13 changes form the largest subgroup with structural chromosome aberrations (Belge et al., 1998). Clonal aberrations of chromosome 2 were likewise frequently found.

In this group the chromosome band p21 was the most frequently found aberration(Bol et al., 1999).

Frequent clonal changes in the chromosome band 2p21 show that this type of aberration forms its own cytogenetic subgroup in benign thyroid gland tumors. The molecular-genetic background of this aberration has not been known to date. Two cell lines with 2p21-translocations from benign thyroid gland tumors were established for identifying the break point and these cell lines and two primary tumors for FISH examinations were used with BAC clones from the chromosome band 2p21. The results of the FISH analyses show that the break points of the examined tumors lie between the BAC clones 339H12 and 1069E24.

In the cell line S533T/SV40 signals were established with none of the two BAC clones on derivative chromosomes. This could possibly be attributed to deletion in this chromosome segment or to excessively short overlapping sequences of both BAC clones, which show insufficiently detectable signals.

The BAC clone 339H12 has a length of 215532 bp and the length of the BAC 1069E24 is estimated as approximately 270 kb according to restriction analyses, since it was not fully sequenced.

The known overlapping sequence of both BAC clones is approximately 45728 bp, so that the maximum size of the break point region is estimated at ca. 450 kb.

Literature

Antonini P, Levy N, Caillou B, Vénuat A M, Schlumberger M, Parmentier C, Bernheim A: Numerical aberrations, including trisomy 22 as the sole anomaly, are recurrent in follicular thyroid adenomas. Genes Chromosome Cancer 8: 63-66 (1993).

Belge G, Bruckmann S, Thode B, Bartnitzke S, Bullerdiek J: Deletions of the short arm of chromosome 2 characterize a new cytogenetic subgroup of benign thyroid tumors. Genes Chromosome Cancer 16: 149-151 (1996).

Belge G, Roque L, Soares J, Bruckmann S, Thode B, Fonseca E, Clode A, Bartnitzke S, Castedo S, Bullerdiek J: Cytogenetic investigations of 340 thyroid hyperplasias and adenomas revealing correlations between cytogenetic findings and histology. Cancer Genet. Cytogenet 101: 42-48 (1998).

Bol S, Belge G, Thode B, Bartnitzke S, Bullerdiek J: Structural abnormalities of chromosome 2 in benign thyroid tumors. Three new cases and review of the literature. Cancer Genet. Cytogenet 114: 75-77 (1999).

Bondeson L, Bengtsson A, Bondeson A. G., Dahlenfors R, Grimelius L, Wedell B, Mark J: Chromosome studies in thyroid neoplasia. Cancer 64: 680-685 (1989).

Bullerdiek J, Böschen C, Bartnitzke S: Aberrations of chromosome 8 in mixed salivary gland tumors: cytogenetic findings on seven cases. Cancer Genet. Cytogenet 24: 205-212 (1987).

Johnson B A, Geha M, Blackwell T K: Similar but distinct effects of the tristetraprolin/TIS 11 immediate-early protein on cell survival. Oncogene 23: 1657-16564 (2000).

ISCN: An International system for Human Cytogenetic Nomenclature. Mitelman F. (ed. ) S. Karger, Basel (1995) Kievits T, Dauwerse J G, Wiegant J, Devilee P, Breuning M H, Cornelisse C. J., van Ommen G J B, Pearson P L: Rapid subchromosomal localization of cosmids by nonradioactive in situ hybridization. Cytogenet. Cell Genet. 53: 134-136 (1990).

Knauff J A, Elisei R, Mochly-Rosen D, Liron T, Chen X N, Gonsky R, Korenberg J R, Fagin J A: Involvement of protein kinase Cs(PKCe) in thyroid cell death. A truncated chimeric PKCs cloned from a thyroid cancer cell line protects thyroid cells from apoptosis. J Biol Chem 274: 23414-23425 (1999).

Roque L, Castedo S, Gomes P, Soares P, Clode A, Soares J: Cytogenetic findings in 18 follicular thyroid adenomas. Cancer Genet. Cytogenet 67: 1-6 (1993).

Sozzi G, Miozzo T, Cariani T C, Bongarzone I, Pilotti S, Pierotti M A, Della Porta G: A t (2; 3) (q 12-13; p 24-25) in follicular thyroid adenomas. Cancer Genet. Cytogenet 64: 38-41 (1992).

Teyssier J R, Ferre D: Frequent clonal chromosomal changes in human non-malignant tumors. Int J Cancer 44: 828-832 (1989).

Teyssier J R, Liautaud-Roger F, Ferre D, Patey M, Dufer J: Chromosomal changes in thyroid tumors.

Relation with DNA content, karyotypic features, and clinical data. Cancer Genet. Cytogenet 50: 249-263 (1990). Wanschura S, Hennig Y, Deichert U, Schoenmakers EFPM, Van de Ven W J M, Bartnitzke S, Bullerdiek J: Molecular-cytogenetic refinement of the 12ql4oqlS breakpoint region affected in uterine leiomyomas. Cytogenet Cell Genst 71: 131-135 (1995).

### EXAMPLE 2

Proof of DRIP and nucleic acids coding for DRIP in primary thyroid gland tumors and cell lines Within the scope of the present example and using the primer and processes concerning RNA isolation characterised further hereinbelow the expression of mRNA was proven in primary thyroid gland tumors and cell lines by means of PCR, whereby the intron/exon boundaries detected by databank sequence comparisons were checked. By sequencing the resulting sequences different fusion gene and splice variants thereof were surprisingly found, which represents the result of the corresponding chromosome translocation.

Standard PCR Conditions (20 μl Preparation)

Template/Matrix: cDNA (2 μl) or genomic DNA (1 μl) or BAC-DNA (1 ng)
10×PCR buffer (−Mg) (Gibco BRL/Invitrogen): 2 μl
primer up/low:1 μl (working solution 10 μmol)
dNTP's: 1 μ1
MgC12+(Gibco BRL/Invitrogen): 0.6 μl
taq polymerase (5 U/μl) (Gibco BRL/Invitrogen): 0.25 μl
Aqua bidest: add 12.15 μl (if cDNA) or add 13.15 μl (if genomic DNA)

Cycles: 35

| | | | Transcript | |
|---|---|---|---|---|
| Primer | Localisation | Template | length | Conditions |
| Fostswi | Exon 38 | HeLa | 130 bp | Prim. denaturing: / |
| 14187/ | Exon 38 | Cell | | 95° C. |
| Rvstswi | | (gen. | | Sec. denaturing: / |
| 14187 | | DNA) | | 95° C. |
| (Acc-No: G21943) | | | | Annealing: 30 sec/ |
| | | | | 56° C. |
| | | | | Extension: 30 sec |
| | | | | 72° C. |
| | | | | Final extension: 72° C. |

Cycler: Eppendorf MastercycIer
Note.:
Gradient of 54° C. to 74° C./Template 2.5 μl

| | | | | |
|---|---|---|---|---|
| Fostswi | Exon 38 | BAC DNA | 130 bp | Prim. denaturing: / |
| 14187/ | Exon 38 | | | 95° C. |
| Rvstswi | | | | Sec. denaturing: / |
| 14187 | | | | 95° C. |
| (Acc- | | | | Annealing: 30 sec/ |
| No: G21943) | | | | 55.5° C. |
| | | | | Extension: 30 sec |
| | | | | 72° C. |
| | | | | Final extension: 72° C. |

Cycler: Eppendorf MastercycIer

| | | | | |
|---|---|---|---|---|
| Fostswi | Exon 38 | MCF 7 | 130 bp | Prim. denaturing: / |
| 14187/ | Exon 38 | Cell | | 95° C. |
| Rvstswi | | (cDNA) | | Sec. denaturing: / |
| 14187 | | | | 95° C. |
| (Acc-No: G21943) | | | | Annealing: 30 sec/ |
| | | | | 55.5° C. |
| | | | | Extension: 30 sec |
| | | | | 72° C. |
| | | | | Final extension: 72° C. |

Cycler: GradientercycIer Biometra
Note.:
Gradient of 50° C. to 70° C.

| | | | | |
|---|---|---|---|---|
| DRIPVRU5/ | Exon 35 | WRO | 483 bp | Prim. denaturing: / |
| DRIPVRL6 | Exon 38 | (cDNA) | | 95° C. |
| | | | | Sec. denaturing: / |
| | | | | 95° C. |
| | | | | Annealing: 30 sec/ |
| | | | | 60.8° C. |
| | | | | Extension: 50 sec |
| | | | | 72° C. |
| | | | | Final extension: 72° C. |

Cycler: Eppendorf MastercycIer
Note.:
Gradient of 50° C. to 70° C.

| | | | | |
|---|---|---|---|---|
| DRIPVRU | Exon 33 | WRO | 162 bp | Prim. denaturing: / |
| DRIPVRL 14 | Exon 34 | (cDNA) | | 95° C. |
| | | | | Sec. denaturing: / |
| | | | | 95° C. |
| | | | | Annealing: 30 sec/ |
| | | | | 55.5° C. |
| | | | | Extension: 30 sec |
| | | | | 72° C. |
| | | | | Final extension: |
| | | | | 72° C. |

Cycler: GradientencycIer Biometra
Note.:
Gradient of 50° C. to 70° C.

| | | | | |
|---|---|---|---|---|
| DRIPVRU11/ | Exon 35 | (BAC | 981 bp | Prim. denaturing: / |
| DRIPVRL12 | Exon 33 | DNA) | | 95° C. |
| | | | | Sec. denaturing: / |
| | | | | 95° C. |
| | | | | Annealing: 30 sec/ |
| | | | | 55.5° C. |
| | | | | Extension: 30 sec |
| | | | | 72° C. |
| | | | | Final extension: |
| | | | | 72° C. |

Cycler: GradientencycIer Biometra
Note.:
Gradient of 50° C. to 70° C.

| | | | | |
|---|---|---|---|---|
| DRIPVRU16/ | Exon 14 | MCF 7 | 820 bp | Prim. denaturing: / |
| DRIPVRL10 | Exon 19 | (cDNA) | | 95° C. |
| | | | | Sec. denaturing: / |
| | | | | 95° C. |
| | | | | Annealing: 30 sec/ |
| | | | | 55.5° C. |
| | | | | Extension: 30 sec |
| | | | | 72° C. |
| | | | | Final extension: 72° C. |

Cycler: GradientencycIer Biometra
Note.:
Gradient of 50° C. to 70° C.

| | | | | |
|---|---|---|---|---|
| DRIPVRU9/ | Exon 18 | MCF 7 | 201 bp | Prim. denaturing: / |
| DRIPVRL10 | Exon 19 | (cDNA) | | 95° C. |
| | | | | Sec. denaturing: / |
| | | | | 95° C. |
| | | | | Annealing: 30 sec/ |
| | | | | 57.3° C. |
| | | | | Extension: 30 sec |
| | | | | 72° C. |
| | | | | Final extension: |
| | | | | 72° C. |

Cycler: GradientencycIer Biometra
Note.:
Gradient of 50° C. to 70° C./Proof at 57.3° C. and 64.4° C.

| | | | | |
|---|---|---|---|---|
| DRIPVRU9/ | Exon 18 | 204D19 | 479 bp | Prim. denaturing: / |
| DRIPVRL10 | Exon 19 | (BAC | | 95° C. |
| | | DNA) | | Sec. denaturing: / |
| | | | | 95° C. |
| | | | | Annealing: 1 min./ |
| | | | | 60° C. |
| | | | | Extension: 45 sec |
| | | | | 72° C. |
| | | | | Final extension: 72° C. |

Cycler: Eppendorf MastercycIer
Note.:
Gradient of 50° C. to 70° C.

| | | | | |
|---|---|---|---|---|
| DRIPVRL8/ | Exon 8/ | MCF 7 | 1105 bp | Prim. denaturing: / |
| DRIPVRU9 | Exon 9 | (cDNA) | | 95° C. |
| | | | | Sec. denaturing: 1/ |
| | | | | 95° C. |
| | | | | Annealing: 1 min./ |
| | | | | 55.5° C. |
| | | | | Extension: 1.5 |
| | | | | 72° C. |
| | | | | Final extension: |
| | | | | 72° C. |

Cycler: Eppendorf MastercycIer
Note.:
Gradient of 50° C. to 70° C.

| | | | | |
|---|---|---|---|---|
| DRIPVRU5/ | Exon 35 | WRO | 764 bp | Prim. denaturing: / |
| DRIPVRL2 | Exon 38 | Cell | | 95° C. |
| | | (cDNA) | | Sec. denaturing: / |
| | | | | 95° C. |
| | | | | Annealing: 1 min./ |
| | | | | 55.5° C. |
| | | | | Extension: 1.5 |
| | | | | 72° C. |
| | | | | Final extension: |
| | | | | 72° C. |

Cycler: Eppendorf MastercycIer
Note.:
Gradient of 50° C. to 70° C.

| | | | | |
|---|---|---|---|---|
| DRIPVRU7/ | Exon 22 | 204D19 | 505 bp | Prim. denaturing: / |
| DRIPVRL8 | Exon 22 | (BAC | | 95° C. |
| | | DNA) | | Sec. denaturing: / |
| | | | | 95° C. |
| | | | | Annealing: 30 sec/ |
| | | | | 60° C. |
| | | | | Extension: 1 min. |
| | | | | 72° C. |
| | | | | Final extension: |
| | | | | 72° C. |

Cycler: GradientencycIer Biometra

PCR I

| | | | |
|---|---|---|---|
| DRIPVRL2/ | Exon 14 | WRO | Prim. denaturing: / |
| DRIPVRU16 | Exon 38 | (cDNA) | 95° C. |
| | | | Sec. denaturing: / |
| | | | 95° C. |
| | | | Annealing: 45 sec/ |
| | | | 65° C. |
| | | | Extension: 4 min. |
| | | | 72° C. |
| | | | Sec. denaturing: |
| | | | 95° C. |
| | | | Annealing: 45 sec |
| | | | 55° C. 10x |
| | | | Extension: 5 min. |
| | | | 72° C. |
| | | | Final extension: 72° C. |

Cycler: GradientencycIer Biometra
Note.:
First PCR for generating the template for subsequent PCR II

PCR II

| | | | | |
|---|---|---|---|---|
| DRIPVRL2/ | Exon 1/ | Template | Ca. 3.5 kb | Prim. denaturing: |
| DRIPVRU16 | Exon 25 | from | | 3 min/ |
| | | PCR I | | 95° C. |
| | | | | Sec. denaturing: / |
| | | | | 95° C. |
| | | | | Annealing: 45 sec/ |
| | | | | 55° C. |
| | | | | Extension: 5 min. |
| | | | | 72° C. |
| | | | | Final extension: |
| | | | | 72° C. |

Cycler: GradientencycIer Biometra

| | | | | |
|---|---|---|---|---|
| DRIPVRU17/ | Exon 18 | cDNA | 880 bp | Prim. denaturing: / |
| DRIPVRL4 | Exon 32 | Clone | | 95° C. |
| | | (DI | | Sec. denaturing: / |
| | | | | 95° C. |
| | | | | Annealing: 45 sec/ |
| | | | | 65° C. |
| | | | | Extension: 1 min. |
| | | | | 72° C. |
| | | | | Final extension: |
| | | | | 72° C. |

Cycler: GradientencycIer Biometra
Note:
cDNA clone from transformation of the PCR product from PCR II

Primer sequences

| | | |
|---|---|---|
| Primer | Sequence (5 → 3′) | Localisat |

| | | |
|---|---|---|
| DRIPVRL2 | TCAACATGCCGCTTCTGTTCTTGGAA.GAGTTAA | Exon 38 |
| DRIPVRL4 | TACTGGGGCAGGCGTGGCACCTTGAAG | Exon 32 |
| DRIPVRU5 | TGGCCGTCGTTGAAGTCCTCACCAGT | Exon 35 |
| DRIPVRL6 | GCCAGCCGGACTTTGAGAGGAGACAGAAG | Exon 38 |
| DRIPVRL8 | CAATGGAACCAGAACATGAAGCCAAGCAGTCT | Exon 22 |
| DRIPVRU9 | GCTTGAGGCAGCAGCAGCATTTCCA | Exon 18 |
| DRIPVRL10 | ATTGGGATGAGGCCTTCAGGGGATGA | Exon 19 |
| DRIPVU11 | CCTGCCCCAGTACCTCCAGAGCCTCAC | Exan 32 |
| DRIPVL12 | GAACTCCTTTGGGGTCAGATGGACACAGTG | Exon 33 |
| DRIPW 13 | ACTGCATGGACCCTGGTGAGTGGCT | Exon 33 |
| DRIPVL14 | GCATGTGGTGGGAAATGACTTTGGAAG | Exan 34 |
| DRIPVU16 | AGAGTA.AATCCAAACGTGAACGAGAGAATGAGT | Exon 14 |
| DRIPVRU17 | | Exon 26 |
| FOSTSWT | ATTTATGTTCAACATGTTTCCTGC | Exon 38 |
| 14187 | | |
| (Acc-No: | | |
| RVSTSWI | TTAACTGCCTCGATTCTGCA | Exon 38 |
| 14187 | | |
| (Acc-No: | | |
| G21943) | | |

Used Cell Lines
MCF-7 (human breast adenocarcinoma)
No:DSM ACC 115
DSMZ (Deutsche Sammlung von Mikroorganismen and Zellkulturen GmbH) [German Collection of Microorganisms and Cell Cultures GmbH]
HELA (human cervix carcinoma)
No: DSM ACC 57
DSMZ (Deutsche Sammlung von Mikroorganismen and Zellkulturen GmbH)
   WRO
BAC 339H12
BAC 204D 19
   Protocol for RNA Isolation
   Treatment with Trizol (Flue!; Blocked Pipette Tips!)
   I. Dissolving Cells

Fully suction medium from the cell culture bottle (Impfbank); wipe out flue with ethanol (70%); prepare 1.5 ml cup (sterile cups, not autoclaved); add 1 ml Trizol directly to the cell culture bottle; suspend the cells by pipetting up and down; transfer cell suspension by pipette to the cup; store cells at −80° C.; place used pipette tips in autoclave waste; leave cell culture bottles to flash into steam under the flue.

II. Phase Separation

Pre-heat water bath to 30° C.; cool centrifuge to 4° C.; thaw cell suspension (and) incubation at RT for 5 min; add 200 μl chloroform per ml Trizol; shake suspension strongly; incubation in water bath at 30° C. for 2-3 min; centrifuging at 12ooo xg and 4° C. for 15 min.

III. RNA Precipitation

Transfer upper phase (RNA!) by pipette to a fresh sterile 1.5 ml cup; add 500 μl iso-amylalcohol (isopropanol); spin carefully; incubation in water bath at 30° C. for 10 min; centrifuging at 12ooo xg and 4° C. for 10 min; then IMMEDIATELY remove excess length by pipette; phenol waste (!).

IV. Washing the RNA

Add 1 ml ethanol (75%) to the pellet—vortex briefly; centrifuging at 7500 xg and 4° C. for 5 min; then IMMEDIATELY remove excess length by pipette; phenol waste (!); leave pellet to dry at RT under the flue for at least 10 min; set water bath at 55° C. (or 60° C.).

V. Dissolving the RNA

Add 30-50 μl sterile dH20 (sterile), depending on size of the pellet; incubation in water bath at 55° C. for 10 min; then place samples immediately on ice; store at −80° C. (or determining concentration).

VI. Determining Concentration

Place 60 μl sterile sdH20 in plastic cuvette; measure empty value; add 1 μl RNA—mix; determine concentration at 260/280 nm+quotient; store RNA at −80° C.

Fish

Carrying out FISH is described for example in Kieviets et al. (Kievits T, Dauwerse J. G., Wiegant J., Devilee P.,

Breuning M. H., Cornelisse C. J., van Ommen G. J. B., Pearson P. L.: Rapid subchromosomal localization of cosmids by nonradioactive in situ hybridization. Cytogenet Cell Genet. 53: 134-136(1990)).

3′-RACE-RT-PCR conditions of S533/TSV40 for proof of the DRIP-FUS2 gene

Template name: cDNA obtained from purifying the total RNA of cells of the cell line S533T/SV40 after reverse transcriptase reaction with AP primer (poly T plus UAP-2 partial sequence)

| Upper | Lower Primer |
|---|---|
| DRIPVRU 19 | UAP-2 (5′- |
| | CUACUACUA- |
| | CUAAAG- |
| | GATCCGTCGA- |

| | Volume | |
|---|---|---|
| | (concentration) | |
| Template | 1.5 | μl |
| 10 × Advantage 2 PCR | 5 | μl |
| buffer | | |
| Upper Primer | 1 | μl |
| (10 μM) | | |
| Lower Primer | 1 | μl |
| (10 μM) | | |
| dNTPs | 1 | μl |
| (10 mM; Promega) | | |
| 50 × Advantage 2 | 1 | μl |
| polymerase mix | | |
| bidest. H₂0 | 39.5 | μl |
| Total volume | 50 | μl |

| Step | Time | | Temperatur [symbol] | Cycles |
|---|---|---|---|---|
| 1.1 | 20 | sec. | 95° C. | 5 |
| 1.2 | 5 | min. | 72° C. | |
| 2.1 | 20 | sec. | 94° C. | 5 |
| 2.2 | 10 | sec. | 70° C. | |
| 2.3 | 5 | min. | 72° C. | |
| 3.1 | 20 | sec. | 94° C. | 25 |
| 3.2 | 10 | sec. | 68° C. | |
| 3.3 | 5 | min. | 72° C. | |
| 4 | 3 | min. | 72° C. | 1 |

PCR Conditions for Probe Manufacture (DRIP-FUS2)
Template description: EIL3500 (cDNA clone; from PCR with DRIPVU16/DRIPVRL2)

Product length: 201 bp

| Upper | Lower Primer |
|---|---|
| DRIPVRU 19 | DRIPVRL 4 |

| | Volume | |
|---|---|---|
| | (concentration) | |
| Template (1:100) | 1 | μl |
| 10 × PCR RxN buffer; MgCl₂ | 2 | μl |
| (Invitrogen) | | |
| Upper Primer | 1 | μl |
| (10 μM) | | |
| Lower Primer | 1 | μl |
| (10 μM) | | |
| dNTPs* | 1 | μl |
| MgCl₂ | 0.6 | μl |
| (50 mM; Invitrogen) | | |
| Taq DNA polymerase | 0.25 | μl |
| recombinant (5 U/μl; | | |
| bidest. H₂0 | 13.15 | μl |
| Total volume | 20 | μl |

*dNTP-Mix: 1.5 μl DIG-11-dUTP alkali labile (25 nM; Roche) + 3 μl dNTPs (10 mM)

| Step | Time | | Temperatur [symbol] | Cycles |
|---|---|---|---|---|
| 1.1 | 3 | min. | 95° C. | 1 |
| 2.1 | 45 | sec. | 95° C. | 35 |
| 2.2 | 45 | sec. | 58° C. | |
| 2.3 | 60 | sec. | 72° C. | |
| 3 | 10 | min. | 72° C. | 1 |

Purifying of Probe DNA with PCR Purification Kit (Qiagen)

3′-RACE-RT-PCR conditions for S325/TSV40 for proof of the DRIP-FUS1 gene
Template description: S325T/SV40 (cDNA)

Type of upper primer and lower primer and reaction preparation identical as for 3′-RACE-RT-PCR for S325T/SV 40

| Step | Time | | Temperatur [symbol] | Cycles |
|---|---|---|---|---|
| 1.1 | 60 | sec. | 94° C. | 1 |
| 2.1 | 30 | sec. | 94° C. | 35 |
| 2.2 | 6 | min. | 68° C. | |
| 3 | 6 | min. | 68° C. | 1 |

PCR Conditions for Probe Manufacture (DRIP-FUS1)
Template description: EIL3500 (cDNA clone; from PCR with DRIPVU16/DRIPVRL2)

Product length: 201 bp

| Upper | Lower Primer |
|---|---|
| DRIPVRU9 | DRIPVRL10 |

| | Volume | |
|---|---|---|
| | (concentration) | |
| Template (1:100) | 1 | μl |
| 10 × PCR RxN buffer; MgCl₂ | 5 | μl |
| (Invitrogen) | | |
| Upper Primer | 1 | μl |
| (10 μM) | | |
| Lower Primer | 1 | μl |
| (10 μM) | | |
| dNTPs* | 1 | μl |
| MgCl₂ | 1.5 | μl |
| (50 mM; Invitrogen) | | |
| Taq DNA polymerase | 0.5 | μl |
| recombinant (5 U/μl; | | |
| bidest. H₂0 | 39 | μl |
| Total volume | 50 | μl |

*dNTP-Mix: 2 μl DIG-11-dUTP alkali labile (25 nM; Roche) + 4 μl dNTPs (10 mM)

| Step | Time | | Temperatur [symbol] | Cycles |
|---|---|---|---|---|
| 1.1 | 3 | min. | 95° C. | 1 |
| 2.1 | 45 | sec. | 95° C. | 35 |
| 2.2 | 45 | sec. | 55° C. | |
| 2.3 | 60 | sec. | 72° C. | |
| 3 | 10 | min. | 72° C. | 1 |

Protocol for Non-Radioactive Hybridising (Southern-Blot) (RACE-PCR DRIP-FUS1 and DRIP-FUS2)
I.
5 min in 2×SSC
2×10 min in depurining buffer
2×15 min in denaturing buffer
3×10 min in neutralising buffer
5 min in 20×SSC
   II.
Start (run warm) vacuum pump at least 15 min prior to blotting
Cut out Whatman filter and Hybond-N membrane (Amersham Pharmacia Biotech) (somewhat larger than the gel)
Assemble blot apparatus (Model 785 vacuum blotter; Biorad)
Attach vacuum (5 inch/mm Hg)
Carefully pour 20×SSC, as soon as the gel is suctioned
Blot for 1-2 h (depending on size/density of the gel)
Every 10 min coat gel with 20×SSC
After the blot:—mark pockets of the gel on the membrane in pencil
Fix DNA on membrane: UV crosslink in Fluo-Link (Biometra) at setting 0.4 t 40 sec.)
   III. Hybridising
Pre-heat express hybridising solution (Clontech) in water bath to 60° C.
Pre-heat hybridising oven to 60° C.
Lie membrane on gauze and roll up from the bottom and place in a hybridising bottle
Add 5 ml Exp-Hyb-solution
Prehybridising for 30 min* at 60° C. in the hybridising oven
Denature probe after 20 min*: 10 min at 95° C. (in PCR block, Biometra) place probe on ice immediately after !
Pipette in Sarstedt tube: vortex 5 ml Exp-Hyb solution +2 μl probe (80 ng/μl)
Place Exp-Hyb solution+probe on membrane in hybridising bottle
Hybridising overnight in hybridising oven at 60° C.
   IV. Wash Membrane
Preparation: 1) pre-heat 0.1×SSC/0.1% SDS in hybridising oven to 60° C., 2) produce buffer 1/1% blocking reagent, plus: mix 90 ml buffer 1+10 ml 10% blocking reagent, whereof: pipette off 10 ml in Sarstedt tube (for point 27)
   Membrane on Agitator for x min in:
2×15 min in 2×SSC/0.1% SDS at RT
2×15 min in 0.1×SSC/0.1% SDS (pre-heated)
5 min in buffer 1/0. 3% Tween-20
30 min in buffer 1/1% blocking reagent
30 min in 10 ml buffer 1/1% blocking reagent +1μ, anti-dig-
AP, FAB fragments (150 U/200. μ1; Enzo)
2×15 min in buffer 1/0, 3% Tween-20
5 min in buffer 3
   V. Proof
Pipette following components together in a 1.5 ml cup
Middle membrane: 1.5 ml buffer 3+15 l CPD-Star (25 mM; Cellmark Diagnostics)
Lie membrane in a film and weld on 3 sides
Add solution directly onto the membrane (on the DNA side)
Weld film without air bubbles and lie in autoradiography cassette DNA side facing up

Further processing in the photo laboratory under red light!
Lie Hyperfilm-ECL (Amersham Pharmacia Biotech) on membrane;
mark corner and identify pockets of the membrane to film
Leave to light 10 min
Leave film in developing bath until it darkens
Place film 10 min in fixing bath
Water film 10 min in water+glacial acetic acid
Dry film in drying cabinet
   Buffer for Southern Blot
   Depurining Buffer
0.25 MHCl [20 mlHCI (37%)]
add. bidest>1000 ml

Denaturing buffer (2000 ml)
0.5 NaOH [40 g NaOH (Mw: 40 g/mol)]
1.5 M NaCl [175.32 gNaCl (Mw: 58.44 g/mol)]
add. bidest>2000 ml
pH 7.4

Neutralising buffer (2000 ml)
1.5M NaCl [175.32 g NaCI (Mw:58.44 g/mol)]
0.5 M Tris-HCl [157.6 g Tris-HCl (Mw: 157.6 g/mol)]
pH 7.4
20×SSC (2000 ml)
3 M NaCl [350.64 g NaCl (Mw: 58.44 g/mol)]
0.3 M Na citrate [176.46 g Na citrate (Mw: 294.1 g/mol)]
add. bidest>2000 ml
pH 7.0

Buffer 1 (2000 ml)
0.1 M maleic acid [23.2 g maleic acid (Mw: 116.07 g/mol)]
0.15 M NaCl [17.4 gNaCl (Mw: 58.44 g/mol)]
15.8 g NaOH [(Mw: 40 g/mol)]
add. bidest >2000 ml
pH 7.5
2×SSC/0.1% SDS(1000 ml)
100ml 20×SSC
5 ml 20% SDS
add. bidest>1000 ml
0.1×SSC/0.1% SDS (1000 ml)
5ml 20×SSC
5 ml 20% SDS
add. bidest>1000 ml

Washing buffer (1000 ml)
buffer 1 [997 ml buffer 1]
0.3% Tween20 [3 ml Tween20]

Blocking stock solution (250 ml)
10% (w/v) blocking reagent [25 g blocking reagent]
in buffer 1 [add. buffer 1 >250 ml]

Buffer 3(1000 ml)
0.1 MTris-HCl [15.76 g Tris-HCl (Mw: 157.6 g/mol)]
0.1 M NaCl [5.84 g NaCl (Mw: 58.44 g/mol)]
add. bidest>1000 ml
pH 9.5
0.4 M NaOH (1000 ml)
16 gNaOH [(Mw: 40 g/mol)]
add. bidest >1000 ml

Strip buffer (1000 ml)
0.1×SSC [5 ml 20×SSC]
0.1% SDS [5 ml 20% SDS]
0.2 MTris-HCl [31.52 g Tris-HCl (Mw: 157.6 g/mol)]
add. bidest>1000 ml
   PCR Proof

Completed DRIP- or DRIP-FUS PCRs

TABLE
Completed DRIP-or DRIP-FUS PCRs an cDNA or genomic DNA

| | Localisation | | Localisation | | | |
|---|---|---|---|---|---|---|
| Upper | Upper Primer | Lower | Lower Primer | | Fragment | |
| Primer | (Exon) | Primer | (Exon) | Template | length | |
| DRIP proof | | | | | | |
| DRIPVU17 | 25 | DRIPVRL4 | 32 | cDNA | 880 | bp |
| DRIPVRU5 | 35 | DRIPVRL2 | 38 | cDNA | 764 | bp |
| DRIPVRU7 | 22_long | DRIPVRL8 | 22_long | cDNA | 505 | bp |
| DRIPVRU7 | 22_long | DRIPVRL8 | 22_long | genomic | 505 | bp |
| | | | | DNA | | |
| DRIPVRU9 | 18 | DR1PVRL10 | 1 | genomic | 479 | bp |
| | | | | DNA | | |
| DRT.PVRU9 | 18 | DRIPVRL10 | 1 | cDNA | 201 | bp |
| DRIPW 11 | 32 | DRIPVL12 | 3 | genomic | 981 | bp |
| | | | | DNA | | |
| DRIPW11 | 32 | DRIPVL12 | 3 | cDNA | 370 | bp |
| DRIPViJll | 32 | DRIPVRL6 | 3 | oDNA | 1049 | bp |
| DRIPW13 | 33 | DRIPVL14 | 3 | cDNA | 162 | bp |
| DRIPV(J16 | 14 | DRIPVRL10 | 1 | cDNA | 820 | bp |
| DRIPW16 | 14 | DRIPVRL2 | 38 | cDNA | ˜3.5 | kb |
| DRIPW19 | 28 | DRIPVRL4 | 32 | cDNA | 560 | bp |
| | | | Exon from | | | |
| DRIP-FUS II proof | | | 7p15 | | | |
| DRIPVU19 | 28 | FUSIIL01 | A | cDNA- | 699 | bp |
| DRIPVU39 | 28 | FUSIIL03 | A | cDNA- | 1142 | bp |

| | | | Exon from | | |
|---|---|---|---|---|---|
| DRIP-FUS I proof | | | 3β25 | | |
| DRIPVUI9 | 28 | FUSVL01 | I | cDNA- | 150 bp/ |
| | | | | | 268 bp |
| DRIPVU19 | 28 | FUSVL03 | I | cDNA- | 165 bp/ |
| | | | | | 283 bp |

PCR Conditions in Detail Define Standard PCR Mix:

| Further designated as | Volume | |
|---|---|---|
| standard PCR mix | (concentration | |
| Template (1:200 diluted) | 1 | μl |
| 10 × PCR RxN buffer; MgCl₂ | 2 | μl |
| (Invitrogen) | | |
| Upper Primer | 1 | μl |
| (10 μM) | | |
| Lower Primer | 1 | μl |
| (10 μM) | | |
| dNTPs | 1 | μl |
| (10 mM; Invitrogen) | | |
| MgCl₂ | 0.6 | μl |
| (50 mM; Invitrogen) | | |
| Taq DNA polymerase | 0.25 | μl |
| recombinant (5 U/μl; | | |
| Bidest. H₂0 | 13.15 | μl |
| Total volume | 20 | μl |

Template description: DIL3500 (cDNA clone; from PCR with DRIPVU16/DRIPVRL2)

Product length: 880 bp

| Upper | Lower |
|---|---|
| Primer | Primer |
| DRIPVRUI7 | DRIPVRL4 |

Standard PCR Mix

Thermocycler:

| Step | Time | | Temperatur [symbol] | Cycles |
|---|---|---|---|---|
| 1 | 3 | min. | 95° C. | 1 |
| 2.1 | 45 | sec. | 95° C. | 35 |
| 2.2 | 45 | sec. | 66° C. | |
| 2.3 | 60 | sec. | 72° C. | |
| 3 | 10 | min. | 72° C. | 1 |

The following result was obtained: main band at 880 bp; a weak band at ˜650 bp; heterogeneous clone rnixture with clones with exon 14 and exon 15 as well as clones without exon 14 and exon 15 (splice variants)

PCR Conditions

Template description: MGF-7 (cDNA)

Product length: 130 bp

| Upper Primer | Lower Primer |
|---|---|
| Rvstswil4187 | Fostswil4187 |

Standard PCR Mix

| Step | Time | | Temperatur [symbol] | Cycles |
|---|---|---|---|---|
| 1 | 3 | min. | 95° C. | 1 |
| 2.1 | 45 | sec. | 95° C. | 35 |
| 2.2 | 30 | sec. | 56° C. | |
| 2.3 | 30 | sec. | 72° C. | |
| 3 | 10 | min. | 72° C. | 1 |

PCR Conditions
Template description: BAC204D19 (genomic DNA)

Product length: 505 bp

| Upper | Lower |
|---|---|
| Primer | Primer |
| DRIPVRU7 | DRIPVRL8 |

Standard PCR Mix

| Step | Time | | Temperatur [symbol] | Cycles |
|---|---|---|---|---|
| 1 | 3 | min. | 95° C. | 1 |
| 2.1 | 45 | sec. | 95° C. | 35 |
| 2.2 | 30 | sec. | 56° C. | |
| 2.3 | 30 | sec. | 72° C. | |
| 3 | 10 | min. | 72° C. | 1 |

PCR Conditions
Template description: HeLa (cDNA)

Product length: 505 bp

| Upper | Lower |
|---|---|
| Primer | Primer |
| DRIPVRU7 | DRIPVRL8 |

Standard PCR Mix

| Step | Time | | Temperature | Cycles |
|---|---|---|---|---|
| 1 | 3 | min. | 95° C. | 1 |
| 2.1 | 45 | sec. | 95° C. | 35 |
| 2.2 | 30 | sec. | 53° C. | |
| 2.3 | 30 | sec. | 72° C. | |
| 3 | 10 | min. | 72° C. | 1 |

PCR Conditions
Template description: BAC204D19 (genomic DNA)

Product length: 479 bp

| Upper | Lower |
|---|---|
| Primer | Primer |
| DRIPVRU9 | DRIPVRL10 |

Standard PCR Mix

| Step | Time | | Temperature | Cycles |
|---|---|---|---|---|
| 1 | 3 | min. | 95° C. | 1 |
| 2.1 | 45 | sec. | 95° C. | 35 |
| 2.2 | 30 | sec. | 56° C. | |
| 2.3 | 45 | sec. | 72° C. | |
| 3 | 10 | min. | 72° C. | 1 |

PCR Conditions
Template description: DIL3500 (cDNA clone; from PCR with DRIPVU16/DRIPVRL2)

Product length: 201 bp

| Upper | Lower |
|---|---|
| Primer | Primer |
| DRIPVRU9 | DRIPVRL10 |

Standard PCR Mix

| Step | Time | | Temperature | Cycles |
|---|---|---|---|---|
| 1 | 3 | min. | 95° C. | 1 |
| 2.1 | 45 | sec. | 95° C. | 35 |
| 2.2 | 45 | sec. | 66° C. | |
| 2.3 | 60 | sec. | 72° C. | |
| 3 | 10 | min. | 72° C. | 1 |

PCR Conditions
Templatep description: MCF 7 (cDNA)

Product length: 201 bp

| Upper | Lower |
|---|---|
| Primer | Primer |
| DRIPVRU9 | DRIPVRL10 |

Standard PCR Mix

| Step | Time | | Temperature | Cycles |
|---|---|---|---|---|
| 1 | 3 | min. | 95° C. | 1 |
| 2.1 | 45 | sec. | 95° C. | 35 |
| 2.2 | 30 | sec. | 56° C. | |
| 2.3 | 30 | sec. | 72° C. | |
| 3 | 10 | min. | 72° C. | 1 |

PCR Conditions
Template description: S325T/SV40 (cDNA)

Product length: 201 bp

| Upper | Lower |
|---|---|
| Primer | Primer |
| DRIPVRU9 | DRIPVRL10 |

Standard PCR Mix

| Step | Time | | Temperature | Cycles |
|---|---|---|---|---|
| 1 | 3 | min. | 95° C. | 1 |
| 2.1 | 45 | sec. | 95° C. | 35 |
| 2.2 | 45 | sec. | 55° C. | |
| 2.3 | 60 | sec. | 72° C. | |
| 3 | 10 | min. | 72° C. | 1 |

PCR Conditions
Template description: S325T/SV40 (cDNA)

Product length: 201 bp

| Upper | Lower |
|---|---|
| Primer | Primer |
| DRIPVRU9 | DRIPVRL10 |

Standard PCR Mix

| Step | Time | | Temperature | Cycles |
|---|---|---|---|---|
| 1 | 3 | min. | 95° C. | 1 |
| 2.1 | 45 | sec. | 95° C. | 35 |
| 2.2 | 45 | sec. | 55° C. | |
| 2.3 | 60 | sec. | 72° C. | |
| 3 | 10 | min. | 72° C. | 1 |

PCR Conditions
Template description: BAC 339H12 (genomic DNA)

Product length: 981 bp

| Upper | Lower |
|---|---|
| Primer | Primer |
| DRIPVRU11 | DRIPVRL12 |

Standard PCR Mix

| Step | Time | | Temperature | Cycles |
|---|---|---|---|---|
| 1 | 3 | min. | 95° C. | 1 |
| 2.1 | 45 | sec. | 95° C. | 35 |
| 2.2 | 30 | sec. | 57° C. | |
| 2.3 | 30 | sec. | 72° C. | |
| 3 | 10 | min. | 72° C. | 1 |

PCR Conditions
Template description: DIL3500 (cDNA clone; from PCR with DRIPVU16/DRIPVRL2)

Product length: 370 bp

| Upper | Lower |
|---|---|
| Primer | Primer |
| DRIPVRU11 | DRIPVRL12 |

Standard PCR Mix

| Step | Time | | Temperature | Cycles |
|---|---|---|---|---|
| 1 | 3 | min. | 95° C. | 1 |
| 2.1 | 45 | sec. | 95° C. | 35 |
| 2.2 | 45 | sec. | 64° C. | |
| 2.3 | 60 | sec. | 72° C. | |
| 3 | 10 | min. | 72° C. | 1 |

PCR Conditions
Template description: WRO (cDNA)

Product length: 1049 bp

| Upper | Lower |
|---|---|
| Primer | Primer |
| DRIPVRU11 | DRIPVRL6 |

Standard PCR Mix

| Step | Time | | Temperatur [symbol] | Cycles |
|---|---|---|---|---|
| 1 | 3 | min. | 95° C. | 1 |
| 2.1 | 60 | sec. | 95° C. | 35 |
| 2.2 | 60 | sec. | 60° C. | |
| 2.3 | 90 | sec. | 72° C. | |
| 3 | 10 | min. | 72° C. | 1 |

PCR Conditions
Template description: MCF-7 (cDNA)

Product length: 162 bp

| Upper | Lower |
|---|---|
| Primer | Primer |
| DRIPVRU13 | DRIPVRL14 |

Standard PCR Mix

| Step | Time | | Temperatur [symbol] | Cycles |
|---|---|---|---|---|
| 1 | 3 | min. | 95° C. | 1 |
| 2.1 | 45 | sec. | 95° C. | 35 |
| 2.2 | 30 | sec. | 56° C. | |
| 2.3 | 30 | sec. | 72° C. | |
| 3 | 10 | min. | 72° C. | 1 |

PCR Conditions
Template description: DIL3500 (cDNA clone; from PCR with
DRIPVU16/DRIPVRL2)

Product length: 820 bp

| Upper | Lower |
|---|---|
| Primer | Primer |
| DRIPVRU16 | DRIPVRL10 |

Standard PCR Mix

| Step | Time | | Temperatur [symbol] | Cycles |
|---|---|---|---|---|
| 1 | 3 | min. | 95° C. | 1 |
| 2.1 | 45 | sec. | 95° C. | 35 |
| 2.2 | 45 | sec. | 52° C. | |
| 2.3 | 60 | sec. | 72° C. | |
| 3 | 10 | min. | 72° C. | 1 |

PCR Conditions
Template description: WRO (cDNA)

Product length: ˜3.5 kbbp

| Upper | Lower |
|---|---|
| Primer | Primer |
| DRIPVRU16 | DRIPVRL2 |

| | |
|---|---|
| PCR 1 | Volume |
| | (Concentration) |

Standard PCR Mix

| Step | Time | | Temperatur [symbol] | Cycles |
|---|---|---|---|---|
| 1 | 3 | min. | 95° C. | 1 |
| 2.1 | 45 | sec. | 95° C. | 5 |
| 2.2 | 30 | sec. | 65° C. | |
| 2.3 | 30 | sec. | 72° C. | |
| 3 | 10 | min. | 72° C. | 1 |
| 4.1 | 45 | sec. | 95° C. | 10 |
| 4.2 | 45 | sec. | 55° C. | |
| 4.3 | 300 | sec. | 72° C. | |

| | |
|---|---|
| Upper | Lower |
| Primer | Primer |
| DRIPVRU16 | DRIPVRL2 |

| | |
|---|---|
| PCR 2 | Volume |
| | (Concentration) |

| Step | Time | | Temperatur [symbol] | Cycles |
|---|---|---|---|---|
| 1 | 3 | min. | 95° C. | 1 |
| 2.1 | 45 | sec. | 95° C. | 35 |
| 2.2 | 45 | sec. | 55° C. | |
| 2.3 | 300 | sec. | 72° C. | |
| 3 | 10 | min. | 72° C. | 1 |

PCR Conditions
Template description: DIL3500 (cDNA clone; from PCR with DRIPVU16/DRIPVRL2)

Product length: 560 bp

| Upper | Lower |
|---|---|
| Primer | Primer |
| DRIPVRU19 | DRIPVRL14 |

Standard PCR Mix

| Step | Time | | Temperatur [symbol] | Cycles |
|---|---|---|---|---|
| 1 | 3 | min. | 95° C. | 1 |
| 2.1 | 45 | sec. | 95° C. | 35 |
| 2.2 | 45 | sec. | 58° C. | |
| 2.3 | 60 | sec. | 72° C. | |
| 3 | 10 | min. | 72° C. | 1 |

PCR Conditions
Template description: S533T/SV40 (cDNA)

Product length: 699 bp

| Upper | Lower |
|---|---|
| Primer | Primer |
| DRIPVRU19 | FUSIILO1 |

Standard PCR Mix

| Step | Time | | Temperatur [symbol] | Cycles |
|---|---|---|---|---|
| 1 | 3 | min. | 95° C. | 1 |
| 2.1 | 45 | sec. | 95° C. | 35 |
| 2.2 | 45 | sec. | 60° C. | |
| 2.3 | 60 | sec. | 72° C. | |
| 3 | 10 | min. | 72° C. | 1 |

PCR Conditions FUS II Fusion Transcript
Template description: S533T/SV40 (cDNA)

Product length: 1142 bp

| Upper | Lower Primer |
|---|---|
| DRIPVRU19 | FUSIIO3 |

| | Volume | |
|---|---|---|
| | (concentration) | |
| Template | 0.5 | μl |
| 10 × PCR R×N buffer; MgCl₂ | 5 | μl |
| (Invitrogen) | | |
| Upper Primer | 1 | μl |
| (10 μM) | | |
| Lower Primer | 1 | μl |
| (10 μM) | | |
| dNTPs | 1 | μl |
| (10 nM; Promega) | | |
| MgCl₂ | 1.5 | μl |
| (50 mM; Invitrogen) | | |
| Taq DNA polymerase | 0.5 | μl |
| recombinant (5 U/μl; | | |
| Bidest. H₂0 | 39.5 | μl |
| Total volume | 50 | μl |

| Step | Time | | Temperatur [symbol] | Cycles |
|---|---|---|---|---|
| 1 | 3 | min. | 95° C. | 1 |
| 2.1 | 45 | sec. | 95° C. | 35 |
| 2.2 | 45 | sec. | 60° C. | |
| 2.3 | 60 | sec. | 72° C. | |
| 3 | 10 | min. | 72° C. | 1 |

PCR Conditions for DRIP-FUS I Fusion Transcript

Template description: S533T/SV40 (cDNA)

| Upper | Lower Primer |
|---|---|
| DRIPVRU19 | FUSVLO1 |

| | Volume | |
|---|---|---|
| | (concentration) | |
| Template | 0.5 | μl |
| 10 × PCR R×N buffer; MgCl₂ | 5 | μl |
| (Invitrogen) | | |
| Upper Primer | 1 | μl |
| (10 μM) | | |
| Lower Primer | 1 | μl |
| (10 μM) | | |
| dNTPs | 1 | μl |
| (10 nM; Promega) | | |
| MgCl₂ | 1.5 | μl |
| (50 mM; Invitrogen) | | |
| Taq DNA polymerase | 0.5 | μl |
| recombinant (5 U/μl; | | |
| Bidest. H₂0 | 39.5 | μl |
| Total volume | 50 | μl |

| Step | Time | | Temperatur [symbol] | Cycles |
|---|---|---|---|---|
| 1 | 3 | min. | 95° C. | 1 |
| 2.1 | 45 | sec. | 95° C. | 35 |
| 2.2 | 45 | sec. | 58° C. | |
| 2.3 | 1 | min. | 72° C. | |
| 3 | 10 | min. | 72° C. | 1 |

PCR Conditions
Template description: S533T/SV40 (cDNA)

Product length: 165 bp (without exon I); 283 bp (incl. exon I)

| Upper | Lower Primer |
|---|---|
| DRIPVRU19 | FUSVLO3 |

| | Volume | |
|---|---|---|
| | (concentration) | |
| Template | 0.5 | μl |
| 10 × PCR R×N buffer; MgCl₂ | 5 | μl |
| (Invitrogen) | | |
| Upper Primer | 1 | μl |
| (10 μM) | | |
| Lower Primer | 1 | μl |
| (10 μM) | | |
| dNTPs | 1 | μl |
| (10 nM; Promega) | | |
| MgCl₂ | 1.5 | μl |
| (50 mM; Invitrogen) | | |
| Taq DNA polymerase | 0.5 | μl |
| recombinant (5 U/μl; | | |
| Bidest. H₂0 | 39.5 | μl |
| Total volume | 50 | μl |

| Step | Time | | Temperatur [symbol] | Cycles |
|---|---|---|---|---|
| 1 | 3 | min. | 95° C. | 1 |
| 2.1 | 45 | sec. | 95° C. | 35 |
| 2.2 | 45 | sec. | 58° C. | |
| 2.3 | 1 | min. | 72° C. | |
| 3 | 10 | min. | 72° C. | 1 |

PCR Conditions
Template description: BAC 339H12 (genomic DNA)

Product length: 130 bp

| Upper Primer | Lower Primer |
|---|---|
| Rvstswi14187 | Fostswi14187 |

Standard PCR Mix

| Step | Time | | Temperatur [symbol] | Cycles |
|---|---|---|---|---|
| 1 | 3 | min. | 95° C. | 1 |
| 2.1 | 45 | sec. | 95° C. | 35 |
| 2.2 | 30 | sec. | 56° C. | |
| 2.3 | 30 | sec. | 72° C. | |
| 3 | 10 | min. | 72° C. | 1 |

PCR Conditions
Template description: HeLa (cDNA)

Product length: 130 bp

| Upper | Lower Primer |
|---|---|
| Rvstswil41 | Fostswil4187 |

| | Volume |
|---|---|
| | (concentration) |
| Template | 2 μl |

| Step | Time | | Temperatur [symbol] | Cycles |
|---|---|---|---|---|
| 1 | 3 | min. | 95° C. | 1 |
| 2.1 | 45 | sec. | 95° C. | 35 |
| 2.2 | 30 | sec. | 56° C. | |
| 2.3 | 30 | sec. | 72° C. | |
| 3 | 10 | min. | 72° C. | 1 |

PCR Conditions
Template description: WRO (cDNA)

Product length: 764 bp

| Upper | Lower |
|---|---|
| Primer | Primer |
| DRIPVRU5 | DRIPVRL2 |

Standard PCR Mix

| Step | Time | | Temperatur [symbol] | Cycles |
|---|---|---|---|---|
| 1 | 3 | min. | 95° C. | 1 |
| 2.1 | 45 | sec. | 95° C. | 35 |
| 2.2 | 30 | sec. | 56° C. | |
| 2.3 | 30 | sec. | 72° C. | |
| 3 | 10 | min. | 72° C. | 1 |

The aspects of the invention, embodiments thereof as well as characteristics of the invention disclosed in the abovementioned description, the sequence protocol and the claims can be essential both individually and in any combination for realising the invention in its different embodiments. The disclosure content of the claims is referred to herewith.

1. A nucleic acid with expression altered in hyperplasias and/or tumors, characterised in that it comprises a nucleic acid sequence, selected from the group which comprises SEQ. ID. No. 1, 3, 4, 9 and SEQ. ID. No. 11 to 16 and in each case fragments thereof.

2. A nucleic acid with expression altered in hyperplasias and/or tumors, characterised in that it comprises a nucleic acid sequence, selected from the group which comprises SEQ. ID. No. 1, 3, 4, 9 and SEQ. ID. No. 11 to 16.

3. The nucleic acid as claimed in claim 1 or 2, characterised in that the tumor is selected from the group, which comprises epithelial tumors with a change to the chromosomal arm 2p and tumors with a change to the chromosomal band 2p21-22.

4. The nucleic acid of claim 1, characterised in that the hyperplasia is selected from the group comprising hyperplasias of the thyroid gland.

5. The nucleic acid of claim 1, characterised in that the nucleic acid is selected from a nucleic acid according to SEQ. ID. No. 1, 9, 13, 14 and/or 15.

6. The nucleic acid comprising a nucleic acid sequence, which without die degeneration of the genetic code would code for the same amino acid sequence as a nucleic acid of claim 1.

7. The nucleic acid, which hybridises to one of the nucleic acids of claim 1, in particular under stringent conditions.

8. A vector, characterised in that it comprises a nucleic acid of claim 1.

9. The vector as claimed in claim 8, characterised in that it further comprises at least one element, which is selected from the group, which comprises promoters, terminators and enhancers.

10. The vector as claimed in claim 8, characterised in that the vector is an expression vector.

11. The vector as claimed in claim 8, characterised in that at least one promoter is in the in-frame with at least part of a nucleic acid of claim 1 coding for a polypeptide.

12. A polypeptide coded by a nucleic acid of claim 1 or with an amino acid sequence according to SEQ. ID. No. 2 or 5 to 8, 10 or 17.

13. The polypeptide as claimed in claim 12, characterised in that it is modified.

14. A cell, in particular an isolated cell, which comprises a vector as claimed in claim 8.

15. An antibody, characterised in that it is directed against a polypeptide as claimed in claim 12.

16. The antibody as claimed in claim 15, characterised in that it is directed against a nucleic acid of claim 1.

17. A ribozyme, characterised in that it is directed against a nucleic acid as claimed in claim 1.

18. The ribozyme as claimed in claim 17, characterised in that it comprises at least a part of a nucleic acid as claimed in claim 1 or of a nucleic acid substantially complementary thereto.

19. An antisense nucleic acid comprising a sequence, which is substantially complementary to or substantially identical to one of the nucleic acid sequences as claimed in claim 1.

20. RNAi comprising a sequence, which is substantially complementary or substantially identical to one of the nucleic acids as claimed in claim 1 or a part thereof, whereby the RNAi preferably comprises a region having a length of 21 to 23 nucleotides, which is substantially complementary or substantially identical.

21. A primer or nucleic acid probe comprising a nucleic acid, whereby the nucleic acid is substantially complementary or substantially identical to one of the nucleic acids as claimed in claim 1 or a part thereof, whereby the primer and/or the nucleic acid probe has a length 12 to 32 nucleotides, preferably 14 to 28 nucleotides and preferably 20 to 28 nucleotides.

22. A method for determining a compound, which influences, in particular inhibits, the effect of a translation product of a nucleic acid as claimed in any one of the preceding claims, characterised by the following steps:
providing the translation product and the compound
contacting the translation product and the compound in a system, which represents the effect of the translation product, and
determining whether a change occurs in the effect of the translation product under the influence of the compound.

23. The method for determining a compound, which influences, in particular inhibits, the effect of a transcription product of a nucleic acid as claimed in any one of the preceding claims, characterised by the following steps:
providing the transcription product and the compound
contacting the transcription product and the compound in a system, which represents the effect of the transcription product, and
determining whether a change occurs in the effect of the transcription product under the influence of the compound.

24. The method as claimed in claim 22 or 23, characterised in that the system is selected from the group, which comprises cellular expression systems, cell-free expression systems, assay for determining the interaction between compound and translation products, and assay for determining the interaction between compound and transcription products.

25. A method for determining genes, responsible for the formation of hyperplasias and tumors, in particular of the thyroid gland, comprising the following steps:
detecting the break points in chromosomal aberrations of the hyperplasias and/or the tumors,
determining genes, which lie inside a region of 400 kbp, preferably 150 kbp, in every direction from the breakpoint region of the chromosome arm 2p, preferably the chromosomal band 2p21-22, and
determining whether the translation/transcription of the gene in a cell of the hyperplasia or of the tumor changes relative to a non-hyperplasia cell or a non-tumor cell.

26. Use of a nucleic acid as claimed in in claim 1 and/or of a ribozyme as claimed in claim 17 and/or an antisense nucleic acid as claimed in claim 19 and/or a RNAi as claimed in claim 20 and/or a nucleic acid probe as claimed in claim 21 for diagnosis, in particular in vitro, and/or therapy of functional disturbances of the thyroid gland and/or hyperplasias of the thyroid gland and/or tumors, in particular thyroid gland tumors.

27. Use of a nucleic acid as claimed in claim 1 and/or of a ribozyme as claimed in claim 17 and/or an antisense nucleic acid as claimed in claim 19 and/or a RNAi as claimed in claim 20 and/or a nucleic acid probe as claimed in claim 21 for the manufacture of a drug, in particular for therapy and/or prevention of functional disturbances of the thyroid gland and/or hyperplasias of the thyroid gland and/or tumors, in particular thyroid gland tumors.

28. Use of a polypeptide as claimed in claim 12 for diagnosis, in particular in vitro, and/or therapy of functional disturbances of the thyroid gland and/or hyperplasias of the thyroid gland and/or tumors, in particular thyroid gland tumors.

29. Use of a polypeptide as claimed in claim 12 for manufacture of a drug, in particular for therapy and/or prevention of functional disturbances of the thyroid gland and/or hyperplasias of the thyroid gland and/or tumors, in particular thyroid gland tumors.

30. Use of an antibody as claimed in claim 15 for diagnosis, in particular in vitro, and/or therapy of functional disturbances of the thyroid gland and/or hyperplasias of the thyroid gland and/or tumors, in particular thyroid gland tumors.

31. Use of an antibody as claimed in claim 15 for the manufacture of a drug, in particular for therapy and/or prevention of functional disturbances of the thyroid gland and/or hyperplasias of the thyroid gland and/or tumors, in particular thyroid gland tumors.

32. A kit for the diagnosis of functional disturbances of the thyroid gland and/or hyperplasias of the thyroid gland and/or thyroid gland tumors, characterised in that the kit comprises at least one element, which is selected from the group comprising a nucleic acid, a vector, a polypeptide, a cell, an antibody, an antisense nucleic acid, RNAi, a ribozyme, a primer and/or a nucleic acid probe, in each case as claimed in any one of the foregoing claims.

33. A method for proving functional disturbances of the thyroid gland and/or hyperplasias of the thyroid gland and/or tumors, in particular tumors of the thyroid gland, characterised by the steps:
contacting thyroid gland material with the agent, which is selected from the group comprising a nucleic acid, a vector, a polypeptide, an antibody, an antisense nucleic acid, RNAi, a ribozyme and a cell, in each case as claimed in any one of the preceding claims, and
determining whether functional disturbances of the thyroid gland and/or hyperplasias of the thyroid gland and/or tumors of the thyroid gland are present.

34. The process as claimed in claim 33, characterised in that the thyroid gland material is present ex vivo.

35. The process as claimed in claim 33, characterised in that the thyroid gland material is part of a cytological preparation.

36. Use of a nucleic acid as claimed in claim 1 or of a part thereof as primer and/or as probe.

37. A primer for displaying and/or screening and/or detecting a nucleic acid, characterised in that it is substantially complementary or substantially identical to a part of a nucleic acid sequence as claimed in claim 1.

38. A method for displaying a nucleic acid, comprising a sequence, which can be detected in thyroid gland tumors or goitre, in which there is an aberration with a fracture point in the chromosomal band 2p21-22, whereby the sequence lies at least partially inside the chromosomal band 2p21-22, characterised in that the process comprises the steps:
providing primers, in particular primers as claimed in claim 35, for carrying out a polymerase chain reaction,
providing a nucleic acid sequence taken from the band 2p21-22 of the human chromosome 2 or a nucleic acid as claimed in any one of claims 1 to 7,
mixing the nucleic acid sequence or the nucleic acid with the primers,
performing a polymerase chain reaction.

39. A pharmaceutical composition, characterised in that it comprises: at least an agent, selected from the group comprising a nucleic acid, a vector, a polypeptide, a cell, an antibody, an antisense nucleic acid, RNAi, a ribozyme, a primer and/or a nucleic acid probe, in each case according to one of the preceding claims, as well as combinations thereof, and at least a pharmaceutically acceptable carrier.

40. A method for the treatment and/or prophylaxis of tumors and hyperplasias, characterised in that a compound is administered to a patient, which prevents the effects of changed expression in nucleic acids as claimed in any one of the preceding claims.

41. Use of a compound, which prevents the effects of changed expression in nucleic acids as claimed in any one of the preceding claims, for manufacturing a drug.

42. Use as claimed in claim 41, characterised in that the drug is for the treatment and/or prophylaxis of tumors and/or hyperplasias, in particular of tumors and/or hyperplasias of the thyroid gland.

43. Use of a nucleic acid having a sequence, whereby the sequence is selected from the group, which comprises SEQ. ID. No. 1, 3, 4, 9 and SEQ. ID. No. 11 to 16, or derivatives thereof and/or polypeptides coded thereby or derivatives thereof for the manufacture of a drug, in particular for the treatment of functional disturbances of the thyroid gland and/or hyperplasias of the thyroid gland and/or thyroid gland tumors and/or for the manufacture of a diagnostic agent, in particular for the diagnosis of functional disturbances of the thyroid gland and/or hyperplasias of the thyroid gland and/or thyroid gland tumors.

44. Use as claimed in claim 43, characterised in that the polypeptide has an amino acid sequence according to No. 2, SEQ. ID. No. 5 to 8, 10 and 17.

45. Use as claimed in claim 43 or 44, characterised in that the nucleic acid would hybridise with the nucleic acid according to one of the SEQ. ID. No. 1, 3, 4, 9 and SEQ. ID. No. 11 to 16 without the degeneration of the genetic code.

46. Use of a polypeptide with a sequence, whereby the sequence is selected from the group, which comprises SEQ. ID. No. 2, SEQ. ID. No. 5 to 8, 10 and 17, or derivatives thereof for the manufacture of a drug, in particular for the treatment of functional disturbances of the thyroid gland and/or hyperplasias of the thyroid gland and/or thyroid gland tumors and/or for the manufacture of a diagnostic agent, in particular for the diagnosis of functional disturbances of the thyroid gland and/or hyperplasias of the thyroid gland and/or thyroid gland tumors.

47. A method for screening a means for the treatment of functional disturbances of the thyroid gland and/or hyperplasias of the thyroid gland and/or tumors, in particular thyroid gland tumors, and/or of a diagnostic agent for the diagnosis of functional disturbances of the thyroid gland and/or hyperplasias of the thyroid gland and/or tumors, in particular thyroid gland tumors, comprising the steps:
providing a candidate compound,
providing an expression system and/or activity system;
contacting the candidate compound with the expression system and/or the activity system;
determining whether, under the influence of the candidate compound, the expression and/or the activity of a nucleic acid with a sequence is changed, whereby the sequence is selected from the group, which comprises SEQ. ID. No. 1, 3, 4, 9 and SEQ. ID. No. 11 to 16, or derivatives thereof and/or polypeptides coded thereby and/or polypeptides having a sequence according to SEQ. ID. 2, SEQ. ID. No. 5 to 8, 10 and 17 derivatives thereof.

48. The method as claimed in claim 47, characterised in that the candidate compound is contained in a compound library.

49. The method as claimed in claim 47 or 48, characterised in that the candidate compound is selected from the group of compound classes, which comprises peptides, proteins, antibodies, anticalins, functional nucleic acids and small molecules.

50. The method as claimed in claim 49, characterised in that the functional nucleic acids are selected from the group, which comprises aptamers, aptazymes, ribozymes, spiegelmers, antisense oligonucleotides and RNAi.

51. Use of a nucleic acid having a sequence, whereby the sequence is selected from the group, which comprises SEQ. ID. No. 1, 3, 4, 9 and SEQ. ID. No. 11 to 16, or derivatives thereof and/or polypeptides coded thereby or derivatives thereof and/or a polypeptide with a sequence according to SEQ. ID. No. 2, SEQ. ID. No. 5 to 8, 10 and 17 or of a derivative thereof and/or of an in particular natural interaction partner of a nucleic acid with a sequence, whereby the sequence is selected from the group, which comprises SEQ. ID. No. 1, 3, 4, 9 and SEQ. ID. No. 11 to 16, or derivatives thereof and/or polypeptides coded thereby or derivatives thereof and/or a nucleic acid coding therefor and/or the interaction partner of a polypeptide with a sequence according to SEQ. ID. No. 2, SEQ. ID. No. 5 to 8, 10 and 17 or of a derivative thereof as target molecule for the development and/or manufacture of a diagnostic agent for diagnosis of functional disturbances of the thyroid gland and/or hyperplasias of the thyroid gland and/or thyroid gland tumors, and/or for the development and/or manufacture of a drug for prevention and/or treatment of functional disturbances of the thyroid gland and/or hyperplasias of the thyroid gland and/or tumors, in particular tumors of the thyroid gland.

52. Use as claimed in claim 51, characterised in that the drug or the diagnostic means comprises an agent, selected from the group, which comprises antibodies, peptides, anticalins, small molecules, antisense molecules, aptamers, spiegelmers and RNAi molecules.

53. Use as claimed in claim 52, characterised in that the agent interacts with a nucleic acid with a sequence, whereby the sequence is selected from the group, which comprises SEQ. ID. No. 1, 3, 4, 9 and SEQ. ID. No. 11 to 16, or derivatives thereof and/or with a nucleic acid coding for a particularly natural interaction partner, interacts in particular with MRNA, genomic nucleic acid or cDNA.

54. Use of a polypeptide, which interacts with a peptide, coded by a nucleic acid with a sequence, whereby the sequence is selected from the group, which comprises SEQ. ID. No. 1, 3, 4, 9 and SEQ. ID. No. 11 to 16, or derivatives thereof and/or a polypeptide according to SEQ. ID. No. 2, SEQ. ID. No. 5 to 8, 10 and 17 and/or interacts with an in particular natural interaction partner thereof for the development or manufacture of a diagnostic means for the diagnosis of functional disturbances of the thyroid gland and/or hyperplasias of the thyroid gland and/or thyroid gland tumors, and/or for the development or manufacture of a drug for prevention and/or treatment of functional disturbances of the thyroid gland and/or hyperplasias of the thyroid gland and/or tumors, in particular thyroid gland tumors.

55. Use as claimed in claim 54, characterised in that the polypeptide is selected from the group, which comprises antibodies and binding polypeptides.

56. Use of a nucleic acid, which interacts with a polypeptide, whereby the polypeptide is coded by a nucleic acid having a sequence, whereby the sequence is selected from the group, which comprises SEQ. ID. No. 1, 3, 4, 9 and SEQ. ID. No. 11 to 16, or derivatives thereof and/or a polypeptide according to SEQ. ID. No. 2, SEQ. ID. No. 5 to 8, 10 and 17 and/or with an in particular natural interaction partner thereof in particular for the development or manufacture of a diagnostic agent for the diagnosis of functional disturbances of the thyroid gland and/or hyperplasias of the thyroid gland and/or thyroid gland tumors, and/or for the development or manufacture of a drug for the prevention and/or treatment of functional disturbances of the thyroid gland and/or hyperplasias of the thyroid gland and/or tumors, in particular thyroid gland tumors.

57. Use as claimed in claim 56, characterised in that the nucleic acid is selected from the group, which comprises aptamers and spiegelmers.

58. Use of a first nucleic acid, which interacts with a second nucleic acid, whereby the second nucleic acid has a sequence, whereby the sequence is selected from the group, which comprises SEQ. ID. No. 1, 3, 4, 9 and SEQ. ID. No. 11 to 16, or derivatives thereof and/or interacts with a nucleic acid, which codes for an interaction partner of a polypeptide with a sequence according to SEQ. ID. No. 2, SEQ. ID. No. 5 to 8, 10 and 17 for the development or manufacture of a drug for the prevention and/or treatment of functional disturbances of the thyroid gland and/or hyperplasias of the thyroid gland and/or tumors, in particular thyroid gland tumors.

59. Use as claimed in claim 58, characterised in that the interacting first nucleic acid is an antisense oligonucleotide, a ribozyme and/or RNAi.

60. Use as claimed in claim 58 or 59, characterised in that the second nucleic acid is the respective cDNA or mRNA.

61. A pharmaceutical composition comprising at least an agent, selected from the group, as defined by the use as claimed in claim 51, and at least a pharmaceutically acceptable carrier, in particular for the prevention and/or treatment of functional disturbances of the thyroid gland and/or hyperplasias of the thyroid gland and/or tumors, in particular thyroid gland tumors.

62. A kit for characterising the status of the thyroid gland, comprising at least an agent, defined by the use as claimed in claim 51.

63. A polypeptide comprising an amino acid sequence according to SEQ. ID. No. 2, SEQ. ID. No. 5 to 8, 10 and 17 or a functional fragment thereof.

64. Use of the polypeptide as claimed in claim 63 according to use as claimed in any one of the foregoing claims.
